(19) United States
(12) Patent Application Publication
(10) Pub. No.: US 2023/0293673 A1
(43) Pub. Date: Sep. 21, 2023

(54) METHODS FOR TREATING AND PREVENTING CYTOMEGALOVIRUS INFECTION

(71) Applicants: La Jolla Institute for Immunology, La Jolla, CA (US); The Board of Supervisors of Louisiana State University and Agricultural and Mechanical College, Baton Rouge, LA (US)

(72) Inventors: Christopher Benedict, La Jolla, CA (US); Jeremy P. Kamil, Shreveport, LA (US); Mohammed Nure Alam Siddiquey, Shreveport, LA (US); Erica Ollmann Saphire, La Jolla, CA (US); Michael Norris, La Jolla, CA (US)

(21) Appl. No.: 18/019,767

(22) PCT Filed: Aug. 6, 2021
(86) PCT No.: PCT/US2021/045079

§ 371 Date: Feb. 3, 2023

Related U.S. Application Data
(60) Provisional application No. 63/062,156, filed on Aug. 6, 2020.

(51) Int. Cl. A61K 39/245; A61P 31/22; A61K 38/16; A61K 38/17; C07K 16/28; C07K 16/08; G01N 33/569; G01N 33/50

(52) U.S. Cl. CPC A61K 39/245; A61P 31/22; A61K 38/162; A61K 38/1793; A61K 38/177; C07K 16/2878; C07K 16/2896; C07K 16/2803; C07K 16/085; G01N 33/56994; G01N 33/5008; C07K 2317/31; C07K 2317/76; C07K 2319/30; G01N 2333/045

(57) ABSTRACT

Provided herein, inter alia, are compositions and methods for treating or preventing viral infections. The methods and compositions may modulate entry of a virus into a cell, viral fusion to a cell, or cell to cell spread of the virus by targeting one or more viral proteins or ligands thereof. The methods and compositions provided herein including embodiments thereof are contemplated to be especially effective for treating or preventing cytomegalovirus infections.

### CROSS-REFERENCE TO RELATED APPLICATIONS

This application claims priority to U.S. Provisional Application No. 63/062,156, filed Aug. 6, 2020, which is hereby incorporated by reference in its entirety and for all purposes.

### REFERENCE TO A SEQUENCE LISTING, A TABLE OR A COMPUTER PROGRAM LISTING APPENDIX SUBMITTED AS AN ASCII TEXT FILE

The Sequence Listing written in file 048513-512001WO_SequenceListing_ST25.TXT, created on Aug. 6, 2021, 327,680 bytes, machine format IBM-PC, MS Windows operating system, is hereby incorporated by reference.

### BACKGROUND OF THE INVENTION

Human cytomegalovirus (HCMV) is a ubiquitous herpesvirus carried by a majority of the human population. Although infection is typically asymptomatic, the virus causes serious disease in persons with compromised or naïve immunity (e.g. transplant and chemotherapy patients)¹. HCMV is also the #1 infectious cause of congenital birth defects resulting from in utero infection, ranging in severity from mild hearing loss to profound neurodevelopmental defects and even death². As of yet there is no approved vaccine, and existing antiviral drugs show undesirable levels of toxicity. Accordingly, HCMV is ranked in the highest priority tier by the National Vaccine Advisory Committee³.

As in all enveloped viruses, HCMV enters cells via fusion of viral and target cell membranes. The core membrane fusion machinery shared across the herpesviruses is comprised of a heterodimer of glycoprotein H (gH)/glycoprotein L (gL), together with glycoprotein B (gB)⁴, where gH/gL is posited to engage host cell surface receptors to regulate gB catalyzed membrane fusion. HCMV gH is a component of two different gH/gL complexes found on the virion envelope. HCMV gL is derivatized in the ER at Cys144, dictating its assembly into gH/gL/gO (trimer) or gH/gL/UL128/UL130/UL131 (pentamer)⁵⁻⁷. These distinct gH/gL complexes can utilize different cell surface receptors, with PDGFRα being a receptor for the trimer⁸⁻¹⁰ and the pentamer utilizing neuropilin-2 (NRP2)¹¹ and OR14I1¹² to enter cells in a CD147-dependent fashion¹³. The trimer is required for the infectivity of cell-free virions^{14,15}, consistent with its expression being stably maintained in HCMV strains repeatedly passaged in fibroblasts. In contrast, virtually all fibroblast-passaged ‘laboratory strains’ acquire mutations in UL128, UL130, or UL131, inactivating the pentamer and abolishing viral tropism for non-fibroblasts; repairing these mutations restores pentamer-dependent infection of epithelial cells, endothelial cells, and leukocytes to varying extents¹⁶⁻¹⁸.

In 2016, a third virion-incorporated, gH-containing complex was reported by GSK Vaccines¹⁹. The reports indicated that a gH/UL116 heterodimer is formed, but unlike gL, UL116 is not disulfide linked to gH. Despite its abundance in virions^{19,20}, a biological role for the gH/UL116 complex has remained elusive.

### BRIEF SUMMARY OF THE INVENTION

UL141, a viral immune-evasion molecule described herein, assembles onto gH/UL116 as a third component of the gH/UL116 complex. Given that gH is a core component of the herpesvirus entry machinery, and that UL141 has been established to bind to and intracellularly sequester TRAIL death receptors of the TNF receptor superfamily, as well as Nectin-2 (CD112, PVRL2) and CD155 (PVR), which play roles in entry of herpes simplex virus and poliovirus, respectively, the presence of UL141 as a component of a gH complex suggests a role in HCMV entry and/or cell-to-cell spread. Inclusion of gH/UL116/UL141 in the context of a live attenuated, subunit, or mRNA vaccine approach may produce more efficacious immune responses to protect against HCMV infection and to limit HCMV disease in various contexts (e.g. congenital disease etc.). Further, compositions and methods targeting one or more components of gH/UL116/UL141, binding partners, or ligands thereof may protect against or treat HCMV infections.

Thus, in an aspect is provided a method of treating or preventing a cytomegalovirus (CMV) infection in a subject in need thereof, the method including administering to the subject an effective amount of an agent that modulates a UL141/UL116/gH multimer.

In an aspect is provided a method of preventing a cytomegalovirus (CMV) infection in a subject in need thereof, the method including administering to the subject a prophylactically effective amount of an agent including UL141 or a fragment thereof.

In another aspect a vaccine composition is provided, the vaccine composition including UL141 or a fragment thereof and a pharmaceutically acceptable excipient.

In another aspect is provided a method for screening agents that inhibit cytomegalovirus (CMV) host cell entry, fusion of CMV with a host cell, or cell-to-cell spread of CMV, including administering a test agent to a CMV-receptive cell, administering CMV to the CMV-receptive cell, and determining whether the agent modulates a UL141/UL116/gH multimer.

In an aspect is provided a composition including an agent coupled to a diagnostic agent, wherein said agent binds a UL141/UL116/gH multimer.

In another aspect a method of diagnosing cytomegalovirus (CMV) infection is provided. The method includes a) contacting a biological sample from a subject with a composition including an agent associated with a diagnostic agent, wherein the agent binds a UL141/UL116/gH multimer; and b) detecting binding of the agent to said UL141/UL116/gH multimer.

In an aspect is provided a composition including the nucleic acid of SEQ ID NO:11, 13, 19, 22, 25, 28, 31, or 33, or a combination thereof.

In an aspect is provided a composition including the nucleic acids provided herein, or the encoded polypeptides.

### BRIEF DESCRIPTION OF THE DRAWINGS

FIG. 1A is a schematic model for the role of UL141 in HCMV entry. A complex between UL141, gH, and UL116 operates in the context of the HCMV virion envelope to regulate the membrane fusion activity of glycoprotein B (gB). In this model, upon ligation of a receptor such as CD155 or TRAIL-R2, UL141 transmits a signal to gB that triggers its conformational rearrangement that is accompanied by fusion of the virion envelope with the cell membrane of the target cell either at the plasma membrane and/or within an intracellular membrane compartment.

FIGS. 1B and 1C illustrate that HCMV clinical strain TR3.1 incorporates UL141 into virions. FIG. 1B and FIG. 1C are representative images of Western blots of indicated viral glycoproteins (UL141, gH, gL, gB) and calnexin (CNX), a cellular marker, comparing infected cell lysates (FIG. 1B) and virions (FIG. 1C), respectively, of BAC-HCMV strains TB40/E and TR3.1. Virus was purified by ultracentrifugation of infected cell supernatants through a 20% D(+) sorbitol cushion. The genome of strain TB40/E contains a spontaneous frameshift in UL141, which occurred during tissue-culture adaptation, while strain TR3.1 contains an intact UL141 gene. Virions were lysed in 1% Triton-X 100 in phosphate buffered saline (pH 7.4) and detergent soluble proteins were analyzed by Western blot for the indicated viral glycoproteins.

FIG. 1D is a representative image of a Western blot showing TR3.1 virion lysates treated with endoglycosidase H (endoH) or protein N-glycosidase F (PNG-F) and analyzed for an immunoreactive band to anti-UL141 serum. Solid black arrows indicate endoH resistant UL141 polypeptides, the white arrow indicates endoH-sensitive UL141 species and fully deglycosylated UL141 after PNGaseF treatment.

FIG. 1E is a representative image of a confocal micrograph of fibroblasts infected with TR3.1 expressing FLAG-tagged UL141. Cells were immuno-stained for gH to label the virion assembly compartment, and for FLAG to detect UL141.

FIG. 2A shows expression of a secreted gH/UL116/UL141 complex from transfected cells. Human embryonic kidney 293T cells were co-transfected with the indicated plasmids expressing UL116 (myc-tagged), the gH ectodomain (FLAG-tagged and lacking its transmembrane anchor), and UL141 (S-tagged, lacking its transmembrane anchor). At 72 hours post transfection, cell supernatants were collected, clarified and subjected to S-protein affinity pulldown or anti-gH immunoprecipitation. Cell lysates and eluted material from supernatant pull downs/immunoprecipitation reactions were analyzed by Western blot for detection of myc, FLAG, S-tag, or beta-actin (actin), as indicated.

FIG. 2B shows representative images of protein gels and Western blots illustrating the detection of viral proteins expressed from plasmids gH_{ecto}^{F}_TBopt+UL116myc_TBopt+UL141ecto_3C His. gH antibodies, myc antibodies and anti-His tag antibodies were used to detect these proteins by Western blot. The proteins were subjected to anti-His tag purification from Expi293F cell supernatant 72 h after transfection. Gels were stained with Coomassie Blue reagent or Ponceau S stain, as indicated.

FIGS. 3A and 3B are representative images of Western blots and a silver stain, illustrating that UL141, gH, and UL116 form a complex in infected cells. FIG. 3A is a representative image of a silver stain and Western blots for detection of UL141 and gH.Anti-gH. Monoclonal antibody clone 14-4b was used to immunoprecipitated gH from cell lysates and the immunoprecipitated material was analyzed. Anti-FLAG M2 agarose beads were used to immunoprecipitate FLAG-tagged proteins and the immunoprecipitated material was analyzed by Western blot. HCMV strain TR3.1 and TR3.1_141^{F}, a virus derived from the HCMV strain TR3.1 BAC in which UL141 is fused at its C-terminus with a FLAG tag, were used to infect fibroblasts. At 72 hpi, cells were lysed and subjected to immunoprecipitation (IP) using anti-FLAG antibody, clone M2. IP eluates were resolved by SDS-PAGE and analyzed by immunoblot and silver stain. FIG. 3B show Western blot analysis. Fibroblasts were either mock-infected or infected with an HCMV strain TB40/E derivative repaired for a frameshift in UL141 (thus restoring UL141 expression) and carrying a myc-epitope tag at the C-terminus of UL116 (r141, UL116^{myc}). At 72 h post-infection cells were lysed and an anti-myc antibody was used to immunoprecipitate myc-tagged proteins. Anti-Myc immunoprecipitates were analyzed by Western blot for the presence of UL116 (myc), gH, gL, and UL141. This illustrates that UL116 associates during infection with glycoprotein H (gH) and UL141, but not glycoprotein L (gLshows Western blot analysis for detection of gH and anti-FLAG immunoreactive protein species.

FIGS. 4A and 4B illustrate UL141 enhances viral multimer-dependent fusion and/or spread in ARPE-19 epithelial cells. FIG. 4A is a schematic of UL128-131 region of AD169rv. UL131 contains a frameshift and is denoted by a box to the left of UL130. Striped overlay indicates portion of genome that is replaced with UL141 in Δ128L r141. FIG. 4B are representative images of ARPE-19 epithelial cells imaged using indirect immunofluorescence (IF) to detect for viral immediate early nuclear antigen IE1-72 kD (IE1) ARPE-19 epithelial cells were infected at an MOI of 0.1 TCID50 per cell with either HCMV strain AD169rv or Δ128L r141. At 10 days postinfection, cells were stained using antibodies against IE1.

FIG. 4C illustrates analysis showing the number of IE1 positive nuclei per plaque as shown in FIG. 4B. Thirty independent plaques from each replicate were counted for the number of IE1 positive nuclei and compared by one-way ANOVA.

FIG. 5 illustrates UL141 binds directly to the ectodomain of TRAIL-R4, in addition to the ectodomains of TRAIL-R1 and -R2. (Top Panel) HEK-293T cells were transfected with a plasmid expressing the UL141 ectodomain fused to a GPI addition signal (UL141-GPI), and its cell surface expression was verified by flow cytometry using an anti-GPI antibody specific for the TAPE repeats. (Bottom Panel) UL141-GPI transfected 293T cells were incubated with varying concentrations of purified TRAIL-R:Fc proteins (R1-R4, ectodomains fused to Ig Fc domain), and cell binding was detected with anti-Fc antibody followed by flow cytometry.

FIG. 6 illustrates binding of m166, a functional orthologue of UL141 encoded by mouse CMV (MCMV) (references 24 and 25) to TRAIL receptors. (Top Panel) HEK-293T cells were transfected with a plasmid expressing the mouse cytomegalovirus m166 ectodomain fused to a GPI addition signal from TRAIL-R3 (UL141-GPI), and cell surface expression was verified by flow cytometry using an anti-GPI antibody specific for the TAPE repeats. (Bottom Panel) m166-GPI expressing 293T cells were incubated with various concentrations of TRAIL-R:Fc recombinant proteins, and binding was detected using an anti-Fc antibody followed by flow cytometry.

FIG. 7 shows that UL141 associates with glycoprotein H (gH) during infection. HCMV strains TR3.1 engineered to carry a FLAG epitope tag at the C-terminus of UL141 (TR3.1_141^{F}) and TB40/E in which a frameshift was repaired to restore a functional UL141 (TB r141) were used to infect human fibroblasts. FIG. 7 shows Western blot analysis of cells to detect the indicated viral proteins, FLAG epitope, or beta-actin (actin). Cells were lysed in non-ionic detergent at 72 h post infection.

FIG. 8 shows generation of 2-mer (UL141/gH dimer) and 3-mer (HCMV UL141/UL116/gH trimer) reactive antibodies. Rabbits (2 each) were immunized with either gH/UL116 (2-mer) or gH/UL116/UL141 (3-mer) purified recombinant proteins, boosted twice at 3 week intervals and then bled to test for reactivity of the polyclonal anti-sera with plate-coated 2-mer or 3-mer protein by ELISA. (Top) Shown is the reactivity of sera from 2 rabbits immunized with the 2-mer, as well as the reactivity of preimmune sera from rabbit #1. (Bottom) Shown is the reactivity of sera from 2 rabbits immunized with the 3-mer, as well as the reactivity of preimmune sera from rabbit #1. As expected, since the 2-mer and 3-mer protein complexes share gH/UL116, polyclonal anti-sera from all rabbits reacts with both, albeit reactivity with the 3-mer tends to be modestly higher.

FIGS. 9A-9C shows low resolution Cryo-EM structure of the HCMV trimer gH/UL116/UL141. FIG. 9A shows the representative 2D class average of gH/UL116/UL141 timer complex. Signal corresponding to each protein is outlined (gH, bottom; UL116, left; UL141, right). FIG. 9B is the low resolution cryo-EM map of HCMV gH/UL116/UL141 trimer complex. Proteins are as labeled. Hypothetical locations of protein stalks are shown as dashed lines FIG. 9C shows the ribbon diagrams of gH (PDB: 7LBE), UL116 (generated with Alphafold2), and UL141 (PDB: 4JMO) docked into the cryo-EM map.

FIGS. 9D-9F show a low resolution Cryo-EM structure of a second conformation of the HCMV trimer gH/UL116/UL141. FIG. 9D shows the representative 2D class average of gH/UL116/UL141 timer complex. Signal corresponding to each protein is outlined (gH, right; UL116, left; UL141, bottom). FIG. 9E Low resolution cryo-EM map of HCMV gH/UL116/UL141 trimer complex. Proteins are as labeled. Hypothetical locations of protein stalks are shown as dashed lines. FIG. 9F are ribbon diagrams of gH (PDB: 7LBE), UL116 (generated with Alphafold2), and UL141 (PDB: 4JMO) docked into the cryo-EM map.

FIG. 10 shows specific binding of the 3-mer (UL141/gH/UL116), but not the 2-mer (UL141/gH), to TRAIL-R2 and TRAIL-R1. 293T cells were transfected with TRAIL-R2:gpi or TRAIL-R1:gpi expression plasmids, and were subsequently incubated with 20 ug/ml of purified recombinant 3-mer (UL141/gH/UL116) or 2-mer protein (gH/UL116), followed by detection with anti-His+2° antibody before analysis by flow cytometry. As expected, only binding of the UL141-containing 3-mer protein showed binding over background levels.

FIGS. 11A and 11B show binding of the 3-mer (UL141/gH/UL116) is detectable with both anti-UL141 and anti-gH. FIG. 11A shows representative histogram analysis of flow cytometry data (left) and the mean fluorescence intensity (MFI) for each concentration of 3-mer protein tested (right). 293T cells were transfected with TRAIL-R2:gpi and incubated with various concentrations of 3-mer protein, followed by detection with anti-His and 2° antibody. Binding was detected by flow cytometry. FIG. 11B shows representative histograms of flow cytometry data. 5 ug/ml of 3-mer was incubated with the same transfected 293T cells (right histogram) or not (left histogram), and binding of 3-mer was detected with anti-gH monoclonal antibody (rabbit monoclonal antibody, clone MSL-109) followed by 2° (anti-Rb IgG-APC). These results show that both gH and UL141 components of the 3-mer can be detected when bound to TRAIL-R2 when using the indicated reagents.

### DETAILED DESCRIPTION OF THE INVENTION

Applicants have found that human cytomegalovirus (HCMV) protein UL141 can form a complex with other HCMV proteins, such as UL116 and gH, to enable/enhance viral entry and/or fusion into host cells, or facilitate cell to cell viral spread. Provided herein are methods of treating or preventing a CMV infection in a subject in need thereof by administering to the subject an effective amount of an agent that modulates a UL141/UL116/gH multimer. Modulation of the UL141/UL116/gH multimer is contemplated to be effective for preventing or inhibiting binding of CMV to a host cell, fusion of CMV with a host cell, cell-to-cell spread of CMV, and/or multimerization of CMV proteins gH, UL116 and UL141, thereby preventing or treating CMV infection. Provided herein are methods and compositions for generating an immune response against UL141 or the UL141/UL116/gH multimer. Also provided are methods for screening agents that inhibit cytomegalovirus (CMV) host cell entry, fusion, and/or cell to cell spread of CMV, including administering a test agent to a CMV-receptive cell, administering CMV to the CMV-receptive cell, and determining whether the agent modulates a UL141/UL116/gH multimer. In embodiments, the CMV is HCMV.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Unless specifically stated or obvious from context, as used herein, the term “or” is understood to be inclusive.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Where substituent groups are specified by their conventional chemical formulae, written from left to right, they equally encompass the chemically identical substituents that would result from writing the structure from right to left, e.g., —CH₂O— is equivalent to —OCH₂—.

As used herein, “small molecule” refers to a low molecular weight compound that may modulate a biological process. A small molecule has a molecular weight of about 900 daltons or less. A small molecule may be a compound that binds specifically to a target biomolecule (e.g. a protein receptor, thus altering the activity or function of the target. In embodiments, the small molecule prevents CMV infection.

As used herein, the term “conjugate” refers to the association between atoms or molecules. The association can be direct or indirect. For example, a conjugate between a nucleic acid and a protein can be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded RNA, and hybrid molecules having mixtures of single and double stranded DNA and RNA. Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In embodiments, the nucleic acid is messenger RNA. In embodiments, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art. In embodiments, the phosphorothioate nucleic acid or phosphorothioate polymer backbone is connected to a detectable label through a chemical linker.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. A nonspecific sequence may be a sequence that does not encode for a functional nucleic acid or protein. In embodiments, a nonspecific sequence is a sequence of a nucleic acid that includes nucleotides randomly attached to each other. In embodiments, a nonspecific sequence does not encode for a biological function. A nonspecific sequence may be referred to as a “scrambled” sequence (e.g., scrambled nucleic acid sequence). A scrambled sequence (e.g., scrambled nucleic acid sequence) may be created by a software tool to create the sequence scramble as negative control for a functional sequence (e.g., nucleic acid sequence). By way of example, a nonspecific sequence (e.g., nucleic acid sequence) is a sequence (e.g., nucleic acid sequence) that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

“Inhibitory nucleic acid” refers to nucleic acids that have the ability to reduce or inhibit expression of a gene or the activity of a target nucleic acid (e.g., a single-stranded or double-stranded RNA or a single-stranded or doubles-stranded DNA) when either expressed in the same cell as the gene or target gene, or when delivered to the cell that has the gene or target gene. In embodiments, an inhibitory nucleic acid is an siRNA or an antisense nucleic acid.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, In embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptide mimetic” or “peptidomimetic” refers to protein-like chain designed to mimic a peptide or protein. Peptide mimetics may be generated by modifying an existing peptide or by designing a compound that mimic peptides, including peptoids and p-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g. a protein complex) associated by noncovalent bonds. The monomers may be substantially identical monomers, or the monomers may be different. In embodiments, the multimer is a dimer (e.g. 2-mer), a trimer (e.g. 3-mer), a tetramer (e.g. 4-mer), or a pentamer (e.g. 5-mer). In embodiments, the multimer is UL141/UL116/gH. Thus, the term “UL141/UL116/gH multimer” refers to the noncovalent association of UL141, UL116 and gH. In embodiments, the multimer is gH/gL/UL128/UL130/UL131.

The term “ligand” is used according to its plain and ordinary meaning in Biochemistry and refers to a molecule that associates with a site on a biomolecule to exert a biological effect. In embodiments, the ligand is a receptor for one or more of UL141, UL116, or gH. In embodiments, the ligand is a molecule that binds a cell-surface receptor.

“Functional portion” or “functional fragment” or “fragment” are interchangeable and refer to a part or a section of a whole molecule that retains at least a portion of the activity of the whole molecule, wherein activity includes binding to other molecules, structural features, signaling, or any other biological effect. In embodiments, the functional portion retains within at least 20%, 30%, 40% 50%, 80%, 90%, 95%, 96%, 97%, 98%, 99% or 100% activity compared to the whole molecule.

The term “splice variant” refers to alternate protein-coding sequences or proteins expressed from said sequences. The splice variant may result from a genetic alteration in the DNA sequence that occurs at the boundary or an exon and an intron (e.g. splice site). The alteration may disrupt RNA splicing, thus resulting in deletion of exons or inclusion of introns, thus resulting in an altered protein-coding sequence. In embodiments, the splice variant is a TRAIL receptor 2 splice variant.

For specific proteins described herein (e.g., TRAIL-R2, U141, U116 etc.), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In embodiments, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference or functional fragment or homolog thereof. In embodiments, the protein is the protein as identified by its UniProt sequence reference. In embodiments, the protein is the protein as identified by its UniProt sequence reference or functional fragment or homolog thereof.

A “UL141” or “UL141 protein” as referred to herein includes any of the recombinant or naturally-occurring forms of Membrane glycoprotein UL141 (UL141) or variants or homologs thereof that maintain UL141 activity (e.g. within at least 50%, 80%, 90%, 95%, 96%, 97%, 98%, 99% or 100% activity compared to UL141). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring UL141 protein. In aspects, the UL141 protein is substantially identical to the protein identified by the UniProt reference number A0A0G2UC50 or a variant or homolog having substantial identity thereto. In aspects, UL141 includes the amino acid sequence of SEQ ID NO:1. In aspects, UL141 has the amino acid sequence of SEQ ID NO:1. In aspects, UL141 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1. In aspects, the UL141 protein has at least 75% sequence identity to the amino acid sequence of SEQ ID NO:1. In aspects, the UL141 protein has at least 80% sequence identity to the amino acid sequence of SEQ ID NO:1. In aspects, the UL141 protein has at least 85% sequence identity to the amino acid sequence of SEQ ID NO:1. In aspects, the UL141 protein has at least 90% sequence identity to the amino acid sequence of SEQ ID NO:1. In aspects, the UL141 protein has at least 95% sequence identity to the amino acid sequence of SEQ ID NO:1.

A “UL116” or “UL116 protein” as referred to herein includes any of the recombinant or naturally-occurring forms of Membrane glycoprotein UL116 (UL116) or variants or homologs thereof that maintain UL116 activity (e.g. within at least 50%, 80%, 90%, 95%, 96%, 97%, 98%, 99% or 100% activity compared to UL116). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring UL116 protein. In aspects, the UL116 protein is substantially identical to the protein identified by the UniProt reference number P16833 or a variant or homolog having substantial identity thereto. In aspects, UL116 includes the amino acid sequence of SEQ ID NO:2. In aspects, UL116 has the amino acid sequence of SEQ ID NO:2. In aspects, UL116 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2. In aspects, the UL116 protein has at least 75% sequence identity to the amino acid sequence of SEQ ID NO:2. In aspects, the UL116 protein has at least 80% sequence identity to the amino acid sequence of SEQ ID NO:2. In aspects, the UL116 protein has at least 85% sequence identity to the amino acid sequence of SEQ ID NO:2. In aspects, the UL116 protein has at least 90% sequence identity to the amino acid sequence of SEQ ID NO:2. In aspects, the UL116 protein has at least 95% sequence identity to the amino acid sequence of SEQ ID NO:2.

A “gH” or “gH protein” as referred to herein includes any of the recombinant or naturally-occurring forms of envelope glycoprotein H (gH) or variants or homologs thereof that maintain gH activity (e.g. within at least 50%, 80%, 90%, 95%, 96%, 97%, 98%, 99% or 100% activity compared to gH). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gH protein. In aspects, the gH protein is substantially identical to the protein identified by the UniProt reference number F5H9T3 or a variant or homolog having substantial identity thereto. In aspects, gH includes the amino acid sequence of SEQ ID NO:3. In aspects, gH has the amino acid sequence of SEQ ID NO:3. In aspects, gH has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:3. In aspects, the gH protein has at least 75% sequence identity to the amino acid sequence of SEQ ID NO:3. In aspects, the gH protein has at least 80% sequence identity to the amino acid sequence of SEQ ID NO:3. In aspects, the gH protein has at least 85% sequence identity to the amino acid sequence of SEQ ID NO:3. In aspects, the gH protein has at least 90% sequence identity to the amino acid sequence of SEQ ID NO:3. In aspects, the gH protein has at least 95% sequence identity to the amino acid sequence of SEQ ID NO:3.

A “gB” or “gB protein” as referred to herein includes any of the recombinant or naturally-occurring forms of Envelope glycoprotein B (gB) or variants or homologs thereof that maintain gB activity (e.g. within at least 50%, 80%, 90%, 95%, 96%, 97%, 98%, 99% or 100% activity compared to gB). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gB protein. In aspects, the gB protein is substantially identical to the protein identified by the UniProt reference number P06473 or a variant or homolog having substantial identity thereto. In aspects, gB includes the amino acid sequence of SEQ ID NO:4. In aspects, gB has the amino acid sequence of SEQ ID NO:4. In aspects, gB has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:4. In aspects, the gB protein has at least 75% sequence identity to the amino acid sequence of SEQ ID NO:4. In aspects, the gB protein has at least 80% sequence identity to the amino acid sequence of SEQ ID NO:4. In aspects, the gB protein has at least 85% sequence identity to the amino acid sequence of SEQ ID NO:4. In aspects, the gB protein has at least 90% sequence identity to the amino acid sequence of SEQ ID NO:4. In aspects, the gB protein has at least 95% sequence identity to the amino acid sequence of SEQ ID NO:4.

A “gL” or “gL protein” as referred to herein includes any of the recombinant or naturally-occurring forms of Envelope glycoprotein L (gL) or variants or homologs thereof that maintain gL activity (e.g. within at least 50%, 80%, 90%, 95%, 96%, 97%, 98%, 99% or 100% activity compared to gL). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gL protein. In aspects, the gL protein is substantially identical to the protein identified by the UniProt reference number P16832 or a variant or homolog having substantial identity thereto. In aspects, gL includes the amino acid sequence of SEQ ID NO:5. In aspects, gL has the amino acid sequence of SEQ ID NO:5. In aspects, gL has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:5. In aspects, the gL protein has at least 75% sequence identity to the amino acid sequence of SEQ ID NO:5. In aspects, the gL protein has at least 80% sequence identity to the amino acid sequence of SEQ ID NO:5. In aspects, the gL protein has at least 85% sequence identity to the amino acid sequence of SEQ ID NO:5. In aspects, the gL protein has at least 90% sequence identity to the amino acid sequence of SEQ ID NO:5. In aspects, the gL protein has at least 95% sequence identity to the amino acid sequence of SEQ ID NO:5.

A “gO” or “gO protein” as referred to herein includes any of the recombinant or naturally-occurring forms of Envelope glycoprotein O (gO) or variants or homologs thereof that maintain gO activity (e.g. within at least 50%, 80%, 90%, 95%, 96%, 97%, 98%, 99% or 100% activity compared to gO). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring gO protein. In aspects, the gO protein is substantially identical to the protein identified by the UniProt reference number F5HGP1 or a variant or homolog having substantial identity thereto. In aspects, gO includes the amino acid sequence of SEQ ID NO:6. In aspects, gO has the amino acid sequence of SEQ ID NO:6. In aspects, gO has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:6. In aspects, the gO protein has at least 75% sequence identity to the amino acid sequence of SEQ ID NO:6. In aspects, the gO protein has at least 80% sequence identity to the amino acid sequence of SEQ ID NO:6. In aspects, the gO protein has at least 85% sequence identity to the amino acid sequence of SEQ ID NO:6. In aspects, the gO protein has at least 90% sequence identity to the amino acid sequence of SEQ ID NO:6. In aspects, the gO protein has at least 95% sequence identity to the amino acid sequence of SEQ ID NO:6.

A “UL128” or “UL128 protein” as referred to herein includes any of the recombinant or naturally-occurring forms of Envelope glycoprotein 128 (UL128) or variants or homologs thereof that maintain UL128 activity (e.g. within at least 50%, 80%, 90%, 95%, 96%, 97%, 98%, 99% or 100% activity compared to UL128). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring UL128 protein. In aspects, the UL128 protein is substantially identical to the protein identified by the UniProt reference number P16837 or a variant or homolog having substantial identity thereto. In aspects, UL128 includes the amino acid sequence of SEQ ID NO:7. In aspects, UL128 has the amino acid sequence of SEQ ID NO:7. In aspects, UL128 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:7. In aspects, the UL128 protein has at least 75% sequence identity to the amino acid sequence of SEQ ID NO:7. In aspects, the UL128 protein has at least 80% sequence identity to the amino acid sequence of SEQ ID NO:7. In aspects, the UL128 protein has at least 85% sequence identity to the amino acid sequence of SEQ ID NO:7. In aspects, the UL128 protein has at least 90% sequence identity to the amino acid sequence of SEQ ID NO:7. In aspects, the UL128 protein has at least 95% sequence identity to the amino acid sequence of SEQ ID NO:7.

A “UL130” or “UL130 protein” as referred to herein includes any of the recombinant or naturally-occurring forms of Envelope glycoprotein 130 (UL130) or variants or homologs thereof that maintain UL130 activity (e.g. within at least 50%, 80%, 90%, 95%, 96%, 97%, 98%, 99% or 100% activity compared to UL130). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring UL130 protein. In aspects, the UL130 protein is substantially identical to the protein identified by the UniProt reference number F5HCP3 or a variant or homolog having substantial identity thereto. In aspects, UL130 includes the amino acid sequence of SEQ ID NO:8. In aspects, UL130 has the amino acid sequence of SEQ ID NO:8. In aspects, UL130 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:8. In aspects, the UL130 protein has at least 75% sequence identity to the amino acid sequence of SEQ ID NO:8. In aspects, the UL130 protein has at least 80% sequence identity to the amino acid sequence of SEQ ID NO:8. In aspects, the UL130 protein has at least 85% sequence identity to the amino acid sequence of SEQ ID NO:8. In aspects, the UL130 protein has at least 90% sequence identity to the amino acid sequence of SEQ ID NO:8. In aspects, the UL130 protein has at least 95% sequence identity to the amino acid sequence of SEQ ID NO:8.

A “UL131” or “UL131 protein” as referred to herein includes any of the recombinant or naturally-occurring forms of Envelope glycoprotein 131 (UL131) or variants or homologs thereof that maintain UL131 activity (e.g. within at least 50%, 80%, 90%, 95%, 96%, 97%, 98%, 99% or 100% activity compared to UL131). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring UL131 protein. In aspects, the UL131 protein is substantially identical to the protein identified by the UniProt reference number P16773 or a variant or homolog having substantial identity thereto. In aspects, UL131 includes the amino acid sequence of SEQ ID NO:9. In aspects, UL131 has the amino acid sequence of SEQ ID NO:9. In aspects, UL131 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:9. In aspects, the UL131 protein has at least 75% sequence identity to the amino acid sequence of SEQ ID NO:9. In aspects, the UL131 protein has at least 80% sequence identity to the amino acid sequence of SEQ ID NO:9. In aspects, the UL131 protein has at least 85% sequence identity to the amino acid sequence of SEQ ID NO:9. In aspects, the UL131 protein has at least 90% sequence identity to the amino acid sequence of SEQ ID NO:9. In aspects, the UL131 protein has at least 95% sequence identity to the amino acid sequence of SEQ ID NO:9.

A “TNF-related apoptosis-inducing ligand receptor” or “TRAIL receptor” as referred to herein includes receptors that bind one or more of TNF-related apoptosis-inducing ligands (TRAIL). TRAIL receptors include TRAIL receptor 1, TRAIL receptor 2, TRAIL receptor 3, TRAIL receptor 4 and Osteoprotegrin (OPG). In embodiments, a TRAIL receptor is a splice variant of a TRAIL receptor provided herein.

A “TRAIL-R2” or “TRAIL-R2 protein” as referred to herein includes any of the recombinant or naturally-occurring forms of TRAIL receptor 2 (TRAIL-R2), also known as Death receptor 5 (DR5), tumor necrosis factor receptor superfamily member 10B (TNFRSF10B), or variants or homologs thereof that maintain TRAIL-R2 activity (e.g. within at least 50%, 80%, 90%, 95%, 96%, 97%, 98%, 99% or 100% activity compared to TRAIL-R2). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRAIL-R2 protein. In aspects, the TRAIL-R2 protein is substantially identical to the protein identified by the UniProt reference number 014763 or a variant or homolog having substantial identity thereto.

In aspects, TRAIL-R2 includes the amino acid sequence of SEQ ID NO:10. In aspects, TRAIL-R2 has the amino acid sequence of SEQ ID NO:10. In aspects, TRAIL-R2 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:10. In aspects, the TRAIL-R2 protein has at least 75% sequence identity to the amino acid sequence of SEQ ID NO:10. In aspects, the TRAIL-R2 protein has at least 80% sequence identity to the amino acid sequence of SEQ ID NO:10. In aspects, the UL131 protein has at least 85% sequence identity to the amino acid sequence of SEQ ID NO:10. In aspects, the TRAIL-R2 protein has at least 90% sequence identity to the amino acid sequence of SEQ ID NO:10. In aspects, the TRAIL-R2 protein has at least 95% sequence identity to the amino acid sequence of SEQ ID NO:10.

In aspects, TRAIL-R2 includes the amino acid sequence of SEQ ID NO:12. In aspects, TRAIL-R2 has the amino acid sequence of SEQ ID NO:12. In aspects, TRAIL-R2 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:12. In aspects, the TRAIL-R2 protein has at least 75% sequence identity to the amino acid sequence of SEQ ID NO:12. In aspects, the TRAIL-R2 protein has at least 80% sequence identity to the amino acid sequence of SEQ ID NO:12. In aspects, the UL131 protein has at least 85% sequence identity to the amino acid sequence of SEQ ID NO:12. In aspects, the TRAIL-R2 protein has at least 90% sequence identity to the amino acid sequence of SEQ ID NO:12. In aspects, the TRAIL-R2 protein has at least 95% sequence identity to the amino acid sequence of SEQ ID NO:12.

In aspects, TRAIL-R2 includes the amino acid sequence of SEQ ID NO:21. In aspects, TRAIL-R2 has the amino acid sequence of SEQ ID NO:21. In aspects, TRAIL-R2 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:21. In aspects, the TRAIL-R2 protein has at least 75% sequence identity to the amino acid sequence of SEQ ID NO:21. In aspects, the TRAIL-R2 protein has at least 80% sequence identity to the amino acid sequence of SEQ ID NO:21. In aspects, the TRAIL-R2 protein has at least 85% sequence identity to the amino acid sequence of SEQ ID NO:21. In aspects, the TRAIL-R2 protein has at least 90% sequence identity to the amino acid sequence of SEQ ID NO:21. In aspects, the TRAIL-R2 protein has at least 95% sequence identity to the amino acid sequence of SEQ ID NO:21.

In aspects, TRAIL-R2 includes the amino acid sequence of SEQ ID NO:24. In aspects, TRAIL-R2 has the amino acid sequence of SEQ ID NO:24. In aspects, TRAIL-R2 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:24. In aspects, the TRAIL-R2 protein has at least 75% sequence identity to the amino acid sequence of SEQ ID NO:24. In aspects, the TRAIL-R2 protein has at least 80% sequence identity to the amino acid sequence of SEQ ID NO:24. In aspects, the TRAIL-R2 protein has at least 85% sequence identity to the amino acid sequence of SEQ ID NO:24. In aspects, the TRAIL-R2 protein has at least 90% sequence identity to the amino acid sequence of SEQ ID NO:24. In aspects, the TRAIL-R2 protein has at least 95% sequence identity to the amino acid sequence of SEQ ID NO:24.

A “CD155” or “CD155 protein” as referred to herein includes any of the recombinant or naturally-occurring forms of cluster of differentiation 155 (CD155), also known as poliovirus receptor, or variants or homologs thereof that maintain CD155 activity (e.g. within at least 50%, 80%, 90%, 95%, 96%, 97%, 98%, 99% or 100% activity compared to CD155). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD155 protein. In aspects, the CD155 protein is substantially identical to the protein identified by the UniProt reference number P15151 or a variant or homolog having substantial identity thereto. In aspects, CD155 includes the amino acid sequence of SEQ ID NO:18. In aspects, CD155 has the amino acid sequence of SEQ ID NO:18. In aspects, CD155 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:18. In aspects, the CD155 protein has at least 75% sequence identity to the amino acid sequence of SEQ ID NO:18. In aspects, the CD155 protein has at least 80% sequence identity to the amino acid sequence of SEQ ID NO:18. In aspects, the UL131 protein has at least 85% sequence identity to the amino acid sequence of SEQ ID NO:18. In aspects, the CD155 protein has at least 90% sequence identity to the amino acid sequence of SEQ ID NO:18. In aspects, the CD155 protein has at least 95% sequence identity to the amino acid sequence of SEQ ID NO:18.

A “TRAIL-R1” or “TRAIL-R1 protein” as referred to herein includes any of the recombinant or naturally-occurring forms of TNF-related apoptosis-inducing ligand receptor 1 (TRAIL-R1), also known as poliovirus receptor, or variants or homologs thereof that maintain TRAIL-R1 activity (e.g. within at least 50%, 80%, 90%, 95%, 96%, 97%, 98%, 99% or 100% activity compared to TRAIL-R1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRAIL-R1 protein. In aspects, the TRAIL-R1 protein is substantially identical to the protein identified by the UniProt reference number 000220 or a variant or homolog having substantial identity thereto. In aspects, TRAIL-R1 includes the amino acid sequence of SEQ ID NO:21. In aspects, TRAIL-R1 has the amino acid sequence of SEQ ID NO:21. In aspects, TRAIL-R1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:21. In aspects, the TRAIL-R1 protein has at least 75% sequence identity to the amino acid sequence of SEQ ID NO:21. In aspects, the TRAIL-R1 protein has at least 80% sequence identity to the amino acid sequence of SEQ ID NO:21. In aspects, the TRAIL-R1 protein has at least 85% sequence identity to the amino acid sequence of SEQ ID NO:21. In aspects, the TRAIL-R1 protein has at least 90% sequence identity to the amino acid sequence of SEQ ID NO:21. In aspects, the TRAIL-R1 protein has at least 95% sequence identity to the amino acid sequence of SEQ ID NO:21.

A “TRAIL-R3” or “TRAIL-R3 protein” as referred to herein includes any of the recombinant or naturally-occurring forms of TNF-related apoptosis-inducing ligand receptor 3 (TRAIL-R3), also known as Antagonist decoy receptor for TRAIL/Apo-2L, Decoy receptor 1, Decoy TRAIL receptor without death domain, or variants or homologs thereof that maintain TRAIL-R3 activity (e.g. within at least 50%, 80%, 90%, 95%, 96%, 97%, 98%, 99% or 100% activity compared to TRAIL-R3). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRAIL-R3 protein. In aspects, the TRAIL-R3 protein is substantially identical to the protein identified by the UniProt reference number 014798 or a variant or homolog having substantial identity thereto. In aspects, TRAIL-R3 includes the amino acid sequence of SEQ ID NO:27. In aspects, TRAIL-R3 has the amino acid sequence of SEQ ID NO:27. In aspects, TRAIL-R3 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:27. In aspects, the TRAIL-R3 protein has at least 75% sequence identity to the amino acid sequence of SEQ ID NO:27. In aspects, the TRAIL-R3 protein has at least 80% sequence identity to the amino acid sequence of SEQ ID NO:27. In aspects, the TRAIL-R3 protein has at least 85% sequence identity to the amino acid sequence of SEQ ID NO:27. In aspects, the TRAIL-R3 protein has at least 90% sequence identity to the amino acid sequence of SEQ ID NO:27. In aspects, the TRAIL-R3 protein has at least 95% sequence identity to the amino acid sequence of SEQ ID NO:27.

A “TRAIL-R4” or “TRAIL-R4 protein” as referred to herein includes any of the recombinant or naturally-occurring forms of TNF-related apoptosis-inducing ligand receptor 4 (TRAIL-R4), also known as Decoy receptor 2 (DCR2), Tumor necrosis factor receptor superfamily member 10D, or variants or homologs thereof that maintain TRAIL-R4 activity (e.g. within at least 50%, 80%, 90%, 95%, 96%, 97%, 98%, 99% or 100% activity compared to TRAIL-R4). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TRAIL-R4 protein. In aspects, the TRAIL-R4 protein is substantially identical to the protein identified by the UniProt reference number Q9UBN6 or a variant or homolog having substantial identity thereto. In aspects, TRAIL-R4 includes the amino acid sequence of SEQ ID NO:30. In aspects, TRAIL-R4 has the amino acid sequence of SEQ ID NO:30. In aspects, TRAIL-R4 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:30. In aspects, the TRAIL-R4 protein has at least 75% sequence identity to the amino acid sequence of SEQ ID NO:30. In aspects, the TRAIL-R4 protein has at least 80% sequence identity to the amino acid sequence of SEQ ID NO:30. In aspects, the TRAIL-R4 protein has at least 85% sequence identity to the amino acid sequence of SEQ ID NO:30. In aspects, the TRAIL-R4 protein has at least 90% sequence identity to the amino acid sequence of SEQ ID NO:30. In aspects, the TRAIL-R4 protein has at least 95% sequence identity to the amino acid sequence of SEQ ID NO:30.

A “CD112” or “CD112 protein” as referred to herein includes any of the recombinant or naturally-occurring forms of CD112, also known as Nectin-2, Herpes virus entry mediator B, Poliovirus receptor-related protein 2, or variants or homologs thereof that maintain CD112 activity (e.g. within at least 50%, 80%, 90%, 95%, 96%, 97%, 98%, 99% or 100% activity compared to CD112). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD112 protein. In aspects, the CD112 protein is substantially identical to the protein identified by the UniProt reference number Q92692 or a variant or homolog having substantial identity thereto. In aspects, CD112 includes the amino acid sequence of SEQ ID NO:32. In aspects, CD112 has the amino acid sequence of SEQ ID NO:32. In aspects, CD112 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:32. In aspects, the CD112 protein has at least 75% sequence identity to the amino acid sequence of SEQ ID NO:32. In aspects, the CD112 protein has at least 80% sequence identity to the amino acid sequence of SEQ ID NO:32. In aspects, the CD112 protein has at least 85% sequence identity to the amino acid sequence of SEQ ID NO:32. In aspects, the CD112 protein has at least 90% sequence identity to the amino acid sequence of SEQ ID NO:32. In aspects, the CD112 protein has at least 95% sequence identity to the amino acid sequence of SEQ ID NO:32.

A “Osteoprotegerin” or “Osteoprotegerin protein” as referred to herein includes any of the recombinant or naturally-occurring forms of Osteoprotegerin, also known as Tumor necrosis factor receptor superfamily member 11B, Osteoclastogenesis inhibitory factor, or variants or homologs thereof that maintain Osteoprotegerin activity (e.g. within at least 50%, 80%, 90%, 95%, 96%, 97%, 98%, 99% or 100% activity compared to Osteoprotegerin). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Osteoprotegerin protein. In aspects, the Osteoprotegerin protein is substantially identical to the protein identified by the UniProt reference number 000300 or a variant or homolog having substantial identity thereto.

The term “modified receptor protein” refers to a receptor protein or a functional portion or fragment thereof that is altered compared to its native state. In embodiments, the modified receptor protein is a receptor protein that is attached to another protein or peptide, wherein the separate protein sequences are recombinantly expressed as a single moiety, or alternatively are chemically linked after expression as separate moieties. In embodiments, the modified receptor protein is linked to one or more therapeutics. In embodiments, the modified receptor protein recognizes UL141, the UL141/UL116/gH multimer, or a multimer including UL141. In embodiments, the modified receptor protein recognizes UL141. In embodiments, the modified receptor protein recognizes the UL141/UL116/gH multimer. In embodiments, the modified receptor protein recognizes a multimer including UL141.

In embodiments, the modified receptor protein has the amino acid sequence of SEQ ID NO:15. In embodiments, the modified receptor protein includes the amino acid sequence of SEQ ID NO:15. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:15. In embodiments, the modified receptor protein has at least 75% sequence identity to the amino acid sequence of SEQ ID NO:15. In embodiments, the modified receptor protein has at least 80% sequence identity to the amino acid sequence of SEQ ID NO:15. In embodiments, the modified receptor protein has at least 85% sequence identity to the amino acid sequence of SEQ ID NO:15. In embodiments, the modified receptor protein has at least 90% sequence identity to the amino acid sequence of SEQ ID NO:15. In embodiments, the modified receptor protein has at least 95% sequence identity to the amino acid sequence of SEQ ID NO:15.

In embodiments, the modified receptor protein has the amino acid sequence of SEQ ID NO:16. In embodiments, the modified receptor protein includes the amino acid sequence of SEQ ID NO:16. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In embodiments, the modified receptor protein has at least 75% sequence identity to the amino acid sequence of SEQ ID NO:16. In embodiments, the modified receptor protein has at least 80% sequence identity to the amino acid sequence of SEQ ID NO:16. In embodiments, the modified receptor protein has at least 85% sequence identity to the amino acid sequence of SEQ ID NO:16. In embodiments, the modified receptor protein has at least 90% sequence identity to the amino acid sequence of SEQ ID NO:16. In embodiments, the modified receptor protein has at least 95% sequence identity to the amino acid sequence of SEQ ID NO:16.

In embodiments, the modified receptor protein has the amino acid sequence of SEQ ID NO:17. In embodiments, the modified receptor protein includes the amino acid sequence of SEQ ID NO:17. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:17. In embodiments, the modified receptor protein has at least 75% sequence identity to the amino acid sequence of SEQ ID NO:17. In embodiments, the modified receptor protein has at least 80% sequence identity to the amino acid sequence of SEQ ID NO:17. In embodiments, the modified receptor protein has at least 85% sequence identity to the amino acid sequence of SEQ ID NO:17. In embodiments, the modified receptor protein has at least 90% sequence identity to the amino acid sequence of SEQ ID NO:17. In embodiments, the modified receptor protein has at least 95% sequence identity to the amino acid sequence of SEQ ID NO:17.

In embodiments, the modified receptor protein has the amino acid sequence of SEQ ID NO:20. In embodiments, the modified receptor protein includes the amino acid sequence of SEQ ID NO:20. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In embodiments, the modified receptor protein has at least 75% sequence identity to the amino acid sequence of SEQ ID NO:20. In embodiments, the modified receptor protein has at least 80% sequence identity to the amino acid sequence of SEQ ID NO:20. In embodiments, the modified receptor protein has at least 85% sequence identity to the amino acid sequence of SEQ ID NO:20. In embodiments, the modified receptor protein has at least 90% sequence identity to the amino acid sequence of SEQ ID NO:20. In embodiments, the modified receptor protein has at least 95% sequence identity to the amino acid sequence of SEQ ID NO:20.

In embodiments, the modified receptor protein has the amino acid sequence of SEQ ID NO:23. In embodiments, the modified receptor protein includes the amino acid sequence of SEQ ID NO:23. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:23. In embodiments, the modified receptor protein has at least 75% sequence identity to the amino acid sequence of SEQ ID NO:23. In embodiments, the modified receptor protein has at least 80% sequence identity to the amino acid sequence of SEQ ID NO:23. In embodiments, the modified receptor protein has at least 85% sequence identity to the amino acid sequence of SEQ ID NO:23. In embodiments, the modified receptor protein has at least 90% sequence identity to the amino acid sequence of SEQ ID NO:23. In embodiments, the modified receptor protein has at least 95% sequence identity to the amino acid sequence of SEQ ID NO:23.

In embodiments, the modified receptor protein has the amino acid sequence of SEQ ID NO:26. In embodiments, the modified receptor protein includes the amino acid sequence of SEQ ID NO:26. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In embodiments, the modified receptor protein has at least 75% sequence identity to the amino acid sequence of SEQ ID NO:26. In embodiments, the modified receptor protein has at least 80% sequence identity to the amino acid sequence of SEQ ID NO:26. In embodiments, the modified receptor protein has at least 85% sequence identity to the amino acid sequence of SEQ ID NO:26. In embodiments, the modified receptor protein has at least 90% sequence identity to the amino acid sequence of SEQ ID NO:26. In embodiments, the modified receptor protein has at least 95% sequence identity to the amino acid sequence of SEQ ID NO:26.

In embodiments, the modified receptor protein has the amino acid sequence of SEQ ID NO:29. In embodiments, the modified receptor protein includes the amino acid sequence of SEQ ID NO:29. In embodiments, the modified receptor protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:29. In embodiments, the modified receptor protein has at least 75% sequence identity to the amino acid sequence of SEQ ID NO:29. In embodiments, the modified receptor protein has at least 80% sequence identity to the amino acid sequence of SEQ ID NO:29. In embodiments, the modified receptor protein has at least 85% sequence identity to the amino acid sequence of SEQ ID NO:29. In embodiments, the modified receptor protein has at least 90% sequence identity to the amino acid sequence of SEQ ID NO:29. In embodiments, the modified receptor protein has at least 95% sequence identity to the amino acid sequence of SEQ ID NO:29.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins. In embodiments, an Fc region may bind TRAIL-R2.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

“Antigen binding fragment” as used herein refers to the region on an antibody that binds to antigens. The antigen binding fragment may refer to the Fab domain of an antibody, but may refer to any portion of the antibody that specifically binds to an antigen epitope. For example, the antigen binding fragment may be an anti-UL141 antibody Fab that binds to an epitope of UL141.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the V_{H} with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

A “bispecific antibody” as provided herein is used according to its conventional meaning well known in the art and refers to a bispecific recombinant protein capable to simultaneously bind to two different antigens. In contrast to traditional monoclonal antibodies, bispecific antibodies consist of two independently different antibody regions (e.g., two single-chain variable fragments (scFv)), each of which binds a different antigen. Similarly, a “trispecific antibody” as provided herein refers to a recombinant protein capable to simultaneously bind to three different antigens.

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulthydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins. In embodiments, the virus is a Cytomegalovirus.

“Cytomegalovirus” or “CMV” refers to the group of related viruses in the Herpesviridae family. Natural hosts of cytomegalovirus include primates, including humans. Species of CMV include, but are not limited to Aotine betaherpesvirus 1, Cebine betaherpesvirus 1, Cercopithecine betaherpesvirus 5, Macacine betaherpesvirus 3 and Panine betaherpesvirus 2. “Human cytomegalovirus” or “HCMV”, also known as Human betaherpesvirus 5 (HHV-5), is a species of cytomegalovirus that infects humans. HCMV comprises a double-stranded DNA genome, a lipid bilayer envelope including various viral glycoproteins, and tegument. Infection by HCMV may be asymptomatic or may cause diseases, including vascular diseases, mononucleosis, and pneumonia, particularly in immunocompromised subjects. In embodiments, the CMV is HCMV.

The term “plaque forming units” is used according to its plain ordinary meaning in Virology and refers to a unit of measurement based on the number of plaques per unit volume of a sample. In some embodiments the units are based on the number of plaques that could form when infecting a monolayer of susceptible cells. Plaque forming unit equivalents are units of measure of inactivated virus. In some embodiments, plaque forming unit equivalents are derived from plaque forming units for a sample prior to inactivation. In embodiments, plaque forming units are abbreviated “Pfu”.

The terms “multiplicity of infection” or “MOI” are used according to its plain ordinary meaning in Virology and refers to the ratio of components (e.g., Paramyxovirus) to the target (e.g., cell) in a given area. In embodiments, the area is assumed to be homogenous.

The term “replicate” is used in accordance with its plain ordinary meaning and refers to the ability of a cell or virus to produce progeny. A person of ordinary skill in the art will immediately understand that the term replicate when used in connection with DNA, refers to the biological process of producing two identical replicas of DNA from one original DNA molecule. In the context of a virus, the term “replicate” includes the ability of a virus to replicate (duplicate the viral genome and packaging said genome into viral particles) in a host cell and subsequently release progeny viruses from the host cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene) relative to the activity of the nuclei acid sequence (e.g., transcription of a gene) in the absence of the composition (e.g., fusion protein, complex, nucleic acid, vector). In embodiments, inhibition refers to reduction of a disease or symptoms of disease (e.g., cancer). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In embodiments, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule. For example, modulating a UL141/UL116/gH multimer refers to changing or varying the activing or function of the UL141/UL116/gH multimer. In embodiments, modulating the UL141/UL116/gH multimer refers to inhibiting the function or activity of the UL141/UL116/gH multimer as to prevent or inhibit binding of CMV to a host cell, fusion of CMV with a host cell, cell-to-cell spread of CMV or multimerization of CMV proteins gH, UL116 and UL141. In embodiments, modulating the UL141/UL116/gH multimer refers to inhibiting formation of the UL141/UL116/gH multimer, inhibiting binding of UL141 to UL116/gH, or inhibiting binding of said multimer to a ligand thereof. In embodiments, the CMV is HCMV.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

“Subject” or “subject in need thereof” refers to a living organism who is at risk of or prone to having a disease or condition, or who is suffering from a disease or condition that can be treated by administration of a composition or pharmaceutical composition as provided herein. For example, the subject may be a subject suffering from HCMV, or symptoms from a HCMV infection. Non-limiting examples include humans, other mammals, bovines, rats, mice, dogs, monkeys, goat, sheep, cows, deer, and other non-mammalian animals. In embodiments, a patient is human. In embodiments, the disease is CMV infection.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognization of a disease or condition by signs and symptoms. Diagnosing can refer to determination of whether a subject has a disease. Diagnosis may refer to determination of the type of disease or condition a subject has.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phoshatase, glucose oxidase, p-galactosidase, p-glucoronidase or p-lactamase. Such enzymes may be used in combination with a chromogen, a fluorogenic compound or a luminogenic compound to generate a detectable signal.

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹n, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Th, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The term “vaccine” refers to a composition that can provide active acquired immunity to and/or therapeutic effect (e.g. treatment) of a particular disease or a pathogen (e.g. CMV, HCMV). A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity. In embodiments, the vaccine reduces, decreases, suppresses, limits, controls, or inhibits CMV viral numbers or titer. In embodiments, the vaccine reduces, decreases, suppresses, limits, controls, or inhibits CMV proliferation or replication. In embodiments, the vaccine reduces, decreases, suppresses, limits, controls, or inhibits the amount of a CMV protein. In embodiments, the vaccine or reduces, decreases, suppresses, limits, controls, or inhibits the amount of CMV viral nucleic acid(s). In embodiments, the vaccine functions by reducing, decreasing, suppressing, limiting, controlling, or inhibiting the function, activity, or ability of CMV to undergo cell-to-cell spread. Thus, in an aspect, the vaccine composition provided herein including embodiments thereof modulates a UL141/UL116/gH multimer. For example, modulating the UL141/UL116/gH multimer may inhibit binding of CMV to a host cell, fusion of CMV with a host cell, cell-to-cell spread of CMV or multimerization of CMV proteins gH, UL116 and UL141, thereby preventing CMV infection. In embodiments, modulating the UL141/UL116/gH multimer may inhibit formation of the UL141/UL116/gH multimer, inhibit binding of UL141 to UL116/gH, or inhibit binding of said multimer to a ligand thereof, thereby preventing CMV infection. In embodiments, the CMV is HCMV.

“Agent” as used herein refers to a composition having the ability to bind a CMV protein or modulate CMV function, including the ability of CMV to fuse with a cell, infect a cell, spread from one cell to another cell, replicate, or exit a cell. For example, the agent may bind an HCMV protein. For example, the agent may modulate HCMV function. Thus, in embodiments the CMV is HCMV. In embodiments, the agent binds to a multimer comprising UL141. In embodiments, the agent may be coupled with a diagnostic agent or a therapeutic agent. In embodiments, the agent can prevent CMV entry into a host cell. In embodiments, the agent can prevent CMV fusion with a host cell. In embodiments, the agent can prevent cell to cell spread of CMV. The agent may be a protein, a nucleic acid, an antibody, an aptamer, a peptidomimetic, a vaccine, or a small molecule. In embodiments, the agent is a protein provided herein, including embodiments thereof. In embodiments, the agent is a nucleic acid encoding said protein. In embodiments, the agent is an antibody or functional fragment thereof that binds to a protein provided herein, including embodiments thereof. In embodiments, the agent is an aptamer. In embodiments, the agent is an aptamer that binds a protein provided herein, including embodiments thereof. In embodiments, the agent is a peptidomimetic. In embodiments, the agent is a peptidomimetic of a protein provided herein, including embodiments thereof. In embodiments, the agent is a vaccine. In embodiments, the agent is a vaccine generated from a protein provided herein, including embodiments thereof. In embodiments, the agent is a nucleic acid encoding a protein provided herein, including embodiments thereof. In embodiments, the agent is a small molecule.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the agents provided herein including embodiments thereof, augments the agent's immune response. Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPILm) (see GB 2220211 (RIBI ImmunoChem Research Inc., Hamilton, Montana, now part of Corixa). Stimulonm QS-21 is a triterpene glycoside or saponin isolated from the bark of the Quillaja Saponaria molina tree found in South America (see Kensil et al., in Vaccine Design: The Subunit and Adjuvant Approach (eds. Powell & Newman, Plenum Press, N Y, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Methods of Treating and Preventing

Although it was long held that functional expression of gH requires its canonical heterodimerization partner gL, gH-containing complexes that lack gL have now been identified in both HCMV and rhesus rhadinovirus (a gamma-2 herpesvirus). Identification of the UL141/gH/UL116 complex described herein allows for development of methods for modulating the function of gL-free gH complex in viral entry and/or fusion and/or cell to cell spread. Moreover, this gH complex presents a previously unappreciated target for urgently needed vaccines to limit congenital HCMV infection, as well as HCMV disease in immunocompromised and/or immunosuppressed contexts such as transplantation, AIDS etc. Work in the last decade showing that the pentameric HCMV envelope glycoprotein complex functions independently of the long-known gH/gL/gO trimeric complex in regulating viral tropism has led to rapid advances with a direct impact on current antiviral drug and vaccine development efforts.

The studies described herein expand understanding of how the diverse repertoire of HCMV glycoproteins govern viral tropism and highlight examples where a viral immunoevasin may play a dual role in HCMV entry and/or fusion. Thus, the methods and compositions provided herein including embodiments thereof are contemplated to be effective for treating and preventing CMV (e.g. HCMV) viral infection.

Thus, in an aspect is provided a method of treating or preventing a cytomegalovirus (CMV) infection in a subject in need thereof, the method including administering to the subject an effective amount of an agent that modulates a UL141/UL116/gH multimer.

In embodiments, the agent modulates (a) binding of CMV to a host cell, (b) fusion of CMV with a host cell, (c) cell-to-cell spread of CMV or (d) oligomerization of CMV proteins gH, UL116 and UL141. In embodiments, the agent modulates binding of CMV to a host cell. In embodiments, the agent modulates fusion of CMV with a host cell. In embodiments, the agent modulates cell-to-cell spread of CMV. In embodiments, the agent modulates multimerization of CMV proteins gH, UL116 and UL141.

In embodiments, the agent (a) inhibits formation of a UL141/UL116/gH multimer (b) inhibits binding of UL141 to UL116/gH, or (c) inhibits binding of said multimer to a ligand thereof. In embodiments, the agent inhibits formation of a UL141/UL116/gH multimer. In embodiments, the agent inhibits binding of UL141 to UL116/gH. In embodiments, the agent inhibits binding of said multimer to a ligand thereof. In embodiments, the agent inhibits viral entry into a cell. In embodiments, the agent inhibits viral fusion to a cell. In embodiments, the agent inhibits cell to cell spread of the virus.

In embodiments, the agent includes one or more of UL141, UL116, gH, gL, gO, UL128, UL130, UL131, or a functional portions thereof. In embodiments, the agent includes UL141, or a functional portion thereof. In embodiments, the agent includes UL116, or a functional portion thereof. In embodiments, the agent includes gH, or a functional portion thereof. In embodiments, the agent includes gL, or a functional portion thereof. In embodiments, the agent includes gO, or a functional portion thereof. In embodiments, the agent includes UL128, or a functional portion thereof. In embodiments, the agent includes UL130, or a functional portion thereof. In embodiments, the agent includes UL131, or a functional portion thereof. In embodiments, the agent includes two or more of any of the proteins provided herein including embodiments thereof, or fragments thereof, noncovalently bound. In embodiments, the agent includes two or more of any of the proteins provided herein including embodiments thereof, or fragments thereof, covalently bound.

In embodiments, the agent includes UL141 or a functional portion thereof. In embodiments, the agent includes a UL141/UL116/gH multimer. In embodiments, the agent further includes gH, gL, gO, UL128, UL130, UL131, or a functional portion thereof. In embodiments, the agent further includes gH, or a functional portion thereof. In embodiments, the agent further includes gL, or a functional portion thereof. In embodiments, the agent further includes gO, or a functional portion thereof. In embodiments, the agent further includes UL128, or a functional portion thereof. In embodiments, the agent further includes UL130, or a functional portion thereof. In embodiments, the agent further includes UL131, or a functional portion thereof.

In embodiments, the agent includes a gH/gL/gO multimer. In embodiments, the agent includes a gH/gL/UL128/UL130/UL131 multimer.

In embodiments, the agent includes a multimer comprising two or more of UL141, UL116, gH, gL, gO, UL128, UL130, UL131, or functional portions thereof.

In embodiments, the agents provided herein including embodiments thereof inhibit formation of a UL141/UL116/gH multimer, disrupt multimerization of UL141/UL116/gH, inhibit binding of UL141 to UL116/gH, inhibit binding of said multimer to a ligand thereof, inhibits viral entry into a cell, inhibits viral fusion to a cell, or inhibits cell to cell spread of the virus.

In embodiments, the agent is a receptor that binds UL141, the UL141/UL116/gH multimer, or a multimer including UL141. In embodiments, the agent is a receptor that binds UL141. In embodiments, the agent is a receptor that binds the UL141/UL116/gH multimer. In embodiments, the agent is a receptor that binds a multimer including UL141. In embodiments, the agent is a TRAIL receptor, CD155, or CD112 or functional portion thereof. In embodiments, the agent is a TRAIL receptor, or a functional portion thereof. In embodiments, the agent is CD155, or a functional portion thereof. In embodiments, the agent is CD112, or a functional portion thereof.

In embodiments, the agent is a TRAIL receptor or a functional portion thereof, wherein the TRAIL receptor or functional portion thereof inhibits formation of a UL141/UL116/gH multimer, disrupts multimerization of UL141/UL116/gH, inhibits binding of UL141 to UL116/gH, inhibits binding of said multimer to a ligand thereof, blocks viral entry into a cell, inhibits viral fusion to a cell, or inhibits cell to cell spread of the virus. In embodiments, the agent is CD155 or a functional portion thereof, wherein CD155 or functional fragment thereof inhibits formation of a UL141/UL116/gH multimer, disrupts multimerization of UL141/UL116/gH, inhibits binding of UL141 to UL116/gH, inhibits binding of said multimer to a ligand thereof, blocks viral entry into a cell, inhibits viral fusion to a cell, or inhibits cell to cell spread of the virus. In embodiments, the agent is CD112 or a functional portion thereof, wherein CD112 or a functional fragment thereof inhibits formation of a UL141/UL116/gH multimer, disrupts multimerization of UL141/UL116/gH, inhibits binding of UL141 to UL116/gH, inhibits binding of said multimer to a ligand thereof, blocks viral entry into a cell, inhibits viral fusion to a cell, or inhibits cell to cell spread of the virus.

In embodiments, the TRAIL receptor is TRAIL receptor 1, TRAIL receptor 2, TRAIL receptor 3, TRAIL receptor 4 or functional portion thereof. In embodiments, the TRAIL receptor is TRAIL receptor 1, or functional portion thereof. In embodiments, the TRAIL receptor is TRAIL receptor 2, or functional portion thereof. In embodiments, the TRAIL receptor is TRAIL receptor 3, or functional portion thereof. In embodiments, the TRAIL receptor is TRAIL receptor 4 or functional portion thereof. In embodiments, the TRAIL receptor is a splice variant of a TRAIL receptor provided herein.

In embodiments, the TRAIL receptor 1 or functional portion thereof inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the TRAIL receptor 2 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the TRAIL receptor 2 or functional portion thereof inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the TRAIL receptor 3 or functional portion inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the TRAIL receptor 4 or functional portion thereof inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the TRAIL receptor is a splice variant of a TRAIL receptor, wherein the TRAIL receptor splice variant inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the TRAIL receptor 2 has the sequence of SEQ ID NO:10, 12, or 24.

In embodiments, the agent is a modified receptor protein. In embodiments, the modified receptor protein includes an Fc domain. In embodiments, the modified receptor protein comprises the sequence of SEQ ID NO:15, 16, 17, 20, 22, 23, 26, 28, or 31. In embodiments, the modified receptor protein comprises the sequence of SEQ ID NO:15. In embodiments, the modified receptor protein comprises the sequence of SEQ ID NO:16. In embodiments, the modified receptor protein comprises the sequence of SEQ ID NO:17. In embodiments, the modified receptor protein comprises the sequence of SEQ ID NO:20. In embodiments, the modified receptor protein comprises the sequence of SEQ ID NO:22. In embodiments, the modified receptor protein comprises the sequence of SEQ ID NO:23. In embodiments, the modified receptor protein comprises the sequence of SEQ ID NO:26. In embodiments, the modified receptor protein comprises the sequence of SEQ ID NO:28. In embodiments, the modified receptor protein comprises the sequence of SEQ ID NO:31. In embodiments, the modified receptor protein inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:15. In embodiments, the modified receptor protein has the sequence of SEQ ID NO:15. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:15. In aspects, the modified receptor protein has a sequence that has at least 85% sequence identity to SEQ ID NO:15. In aspects, the modified receptor protein has a sequence that has at least 90% sequence identity to SEQ ID NO:15. In aspects, the modified receptor protein has a sequence that has at least 95% sequence identity to SEQ ID NO:15. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:15. In aspects, the modified receptor protein has a sequence that has at least 85% sequence identity to SEQ ID NO:15. In aspects, the modified receptor protein has a sequence that has at least 90% sequence identity to SEQ ID NO:15. In aspects, the modified receptor protein has a sequence that has at least 95% sequence identity to SEQ ID NO:15. In embodiments, the modified receptor protein comprising the sequence of SEQ ID NO:15 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:16. In embodiments, the modified receptor protein has the sequence of SEQ ID NO:16. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In aspects, the modified receptor protein has a sequence that has at least 85% sequence identity to SEQ ID NO:16. In aspects, the modified receptor protein has a sequence that has at least 90% sequence identity to SEQ ID NO:16. In aspects, the modified receptor protein has a sequence that has at least 95% sequence identity to SEQ ID NO:16. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In aspects, the modified receptor protein has a sequence that has at least 85% sequence identity to SEQ ID NO:16. In aspects, the modified receptor protein has a sequence that has at least 90% sequence identity to SEQ ID NO:16. In aspects, the modified receptor protein has a sequence that has at least 95% sequence identity to SEQ ID NO:16. In embodiments, the modified receptor protein comprising the sequence of SEQ ID NO:16 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:17. In embodiments, the modified receptor protein has the sequence of SEQ ID NO:17. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:17. In aspects, the modified receptor protein has a sequence that has at least 85% sequence identity to SEQ ID NO:17. In aspects, the modified receptor protein has a sequence that has at least 90% sequence identity to SEQ ID NO:17. In aspects, the modified receptor protein has a sequence that has at least 95% sequence identity to SEQ ID NO:17. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:17. In aspects, the modified receptor protein has a sequence that has at least 85% sequence identity to SEQ ID NO:17. In aspects, the modified receptor protein has a sequence that has at least 90% sequence identity to SEQ ID NO:17. In aspects, the modified receptor protein has a sequence that has at least 95% sequence identity to SEQ ID NO:17. In embodiments, the modified receptor protein comprising the sequence of SEQ ID NO:17 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:20. In embodiments, the modified receptor protein has the sequence of SEQ ID NO:20. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In aspects, the modified receptor protein has a sequence that has at least 85% sequence identity to SEQ ID NO:20. In aspects, the modified receptor protein has a sequence that has at least 90% sequence identity to SEQ ID NO:20. In aspects, the modified receptor protein has a sequence that has at least 95% sequence identity to SEQ ID NO:20. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In aspects, the modified receptor protein has a sequence that has at least 85% sequence identity to SEQ ID NO:20. In aspects, the modified receptor protein has a sequence that has at least 90% sequence identity to SEQ ID NO:20. In aspects, the modified receptor protein has a sequence that has at least 95% sequence identity to SEQ ID NO:20. In embodiments, the modified receptor protein comprising the sequence of SEQ ID NO:20 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:22. In embodiments, the modified receptor protein has the sequence of SEQ ID NO:22. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:22. In aspects, the modified receptor protein has a sequence that has at least 85% sequence identity to SEQ ID NO:22. In aspects, the modified receptor protein has a sequence that has at least 90% sequence identity to SEQ ID NO:22. In aspects, the modified receptor protein has a sequence that has at least 95% sequence identity to SEQ ID NO:22. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:22. In aspects, the modified receptor protein has a sequence that has at least 85% sequence identity to SEQ ID NO:22. In aspects, the modified receptor protein has a sequence that has at least 90% sequence identity to SEQ ID NO:22. In aspects, the modified receptor protein has a sequence that has at least 95% sequence identity to SEQ ID NO:22. In embodiments, the modified receptor protein comprising the sequence of SEQ ID NO:22 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:23. In embodiments, the modified receptor protein has the sequence of SEQ ID NO:23. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:23. In aspects, the modified receptor protein has a sequence that has at least 85% sequence identity to SEQ ID NO:23. In aspects, the modified receptor protein has a sequence that has at least 90% sequence identity to SEQ ID NO:23. In aspects, the modified receptor protein has a sequence that has at least 95% sequence identity to SEQ ID NO:23. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:23. In aspects, the modified receptor protein has a sequence that has at least 85% sequence identity to SEQ ID NO:23. In aspects, the modified receptor protein has a sequence that has at least 90% sequence identity to SEQ ID NO:23. In aspects, the modified receptor protein has a sequence that has at least 95% sequence identity to SEQ ID NO:23. In embodiments, the modified receptor protein comprising the sequence of SEQ ID NO:23 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:26. In embodiments, the modified receptor protein has the sequence of SEQ ID NO:26. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In aspects, the modified receptor protein has a sequence that has at least 85% sequence identity to SEQ ID NO:26. In aspects, the modified receptor protein has a sequence that has at least 90% sequence identity to SEQ ID NO:26. In aspects, the modified receptor protein has a sequence that has at least 95% sequence identity to SEQ ID NO:26. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In aspects, the modified receptor protein has a sequence that has at least 85% sequence identity to SEQ ID NO:26. In aspects, the modified receptor protein has a sequence that has at least 90% sequence identity to SEQ ID NO:26. In aspects, the modified receptor protein has a sequence that has at least 95% sequence identity to SEQ ID NO:26. In embodiments, the modified receptor protein comprising the sequence of SEQ ID NO:26 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein comprises the sequence of SEQ ID NO:28. In embodiments, the modified receptor protein has the sequence of SEQ ID NO:28. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, the modified receptor protein has a sequence that has at least 85% sequence identity to SEQ ID NO:28. In aspects, the modified receptor protein has a sequence that has at least 90% sequence identity to SEQ ID NO:28. In aspects, the modified receptor protein has a sequence that has at least 95% sequence identity to SEQ ID NO:28. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, the modified receptor protein has a sequence that has at least 85% sequence identity to SEQ ID NO:28. In aspects, the modified receptor protein has a sequence that has at least 90% sequence identity to SEQ ID NO:28. In aspects, the modified receptor protein has a sequence that has at least 95% sequence identity to SEQ ID NO:28. In embodiments, the modified receptor protein comprising the sequence of SEQ ID NO:28 inhibits formation of a UL141/UL116/gH multimer, disrupts multimerization of UL141/UL116/gH, inhibits binding of UL141 to UL116/gH, inhibits binding of said multimer to a ligand thereof, or inhibits viral entry into a cell, inhibits viral fusion to a cell, or inhibits cell to cell spread of the virus.

In embodiments, the modified receptor protein comprises the sequence of SEQ ID NO:31. In embodiments, the modified receptor protein has the sequence of SEQ ID NO:31. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:31. In aspects, the modified receptor protein has a sequence that has at least 85% sequence identity to SEQ ID NO:31. In aspects, the modified receptor protein has a sequence that has at least 90% sequence identity to SEQ ID NO:31. In aspects, the modified receptor protein has a sequence that has at least 95% sequence identity to SEQ ID NO:31. In aspects, the modified receptor protein has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:31. In aspects, the modified receptor protein has a sequence that has at least 85% sequence identity to SEQ ID NO:31. In aspects, the modified receptor protein has a sequence that has at least 90% sequence identity to SEQ ID NO:31. In aspects, the modified receptor protein has a sequence that has at least 95% sequence identity to SEQ ID NO:31. In embodiments, the modified receptor protein comprising the sequence of SEQ ID NO:31 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the modified receptor protein includes an Fc domain.

In embodiments, the agent is an antibody or antigen binding fragment. In embodiments, the agent is an antibody. In embodiments, the agent is an antigen binding fragment. In embodiments, the antibody or antigen binding fragment is a human antibody, a monoclonal antibody, a polyclonal antibody, a single chain antibody, Fab, Fab′, F(ab′)2, Fv or scFv. In embodiments, the antibody or antigen binding fragment is a human antibody. In embodiments, the antibody or antigen binding fragment is a monoclonal antibody. In embodiments, the antibody or antigen binding fragment is a polyclonal antibody. In embodiments, the antibody or antigen binding fragment is a single chain antibody. In embodiments, the antibody or antigen binding fragment is a Fab. In embodiments, the antibody or antigen binding fragment is a Fab′. In embodiments, the antibody or antigen binding fragment is a F(ab′)2. In embodiments, the antibody or antigen binding fragment is a Fv. In embodiments, the antibody or antigen binding fragment is a scFv.

In embodiments, the antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the antibody fragment inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the Fab inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the Fab′ inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the F(ab′)2 inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the Fv inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the scFv inhibits formation of a UL141/UL116/gH multimer, disrupts multimerization of UL141/UL116/gH, inhibits binding of UL141 to UL116/gH, inhibits binding of said multimer to a ligand thereof, UL141/UL116/gH.

In embodiments, the antibody is an anti-UL141, anti-TRAIL receptor, anti-CD155, or anti-CD112 antibody. In embodiments, the antibody is an anti-UL141 antibody. In embodiments, the antibody is an anti-TRAIL receptor antibody. In embodiments, the antibody is an anti-CD155 antibody. In embodiments, the antibody is an anti-CD112 antibody. In embodiments, the antibody recognizes a receptor that binds UL141, UL141/UL116/gH, or a multimer including UL141.

In embodiments, the anti-UL141 antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the anti-TRAIL receptor antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the anti-CD155 antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the anti-CD121 antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

In embodiments, the agent is an antibody or antigen binding fragment thereof that recognizes a multimer comprising UL141. In embodiments, the agent is a multispecific antibody that recognizes at least one epitope of UL141. In embodiments, the agent is a bispecific or a trispecific antibody that recognizes at least one epitope of UL141. In embodiments, the agent is a bispecific antibody that recognizes at least one epitope of UL141. In embodiments, the agent is a trispecific antibody that recognizes at least one epitope of UL141.

In embodiments, the multispecific antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the bispecific antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the trispecific antibody inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus.

For the methods provided herein, including embodiments thereof, the agent includes a small molecule, a peptide mimetic, an aptamer, or an inhibitory nucleic acid. In embodiments, the agent includes a small molecule. In embodiments, the agent includes a peptide mimetic. In embodiments, the agent includes an aptamer. In embodiments, the agent includes an inhibitory nucleic acid.

In embodiments, the small molecule, peptide mimetic, aptamer, or inhibitory nucleic acid inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the small molecule inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits viral fusion to a cell, or inhibits cell to cell spread of the virus. In embodiments, the peptide mimetic inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits virus spread between cells, or inhibits viral fusion with a host cell. In embodiments, the aptamer inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits virus spread between cells, or inhibits viral fusion with a host cell. In embodiments, the inhibitory nucleic acid inhibits formation of a UL141/UL116/gH multimer, disrupts multimerization of UL141/UL116/gH, inhibits binding of UL141 to UL116/gH, inhibits binding of said multimer to a ligand thereof, inhibits viral entry into a cell, inhibits virus spread between cells, or inhibits viral fusion with a host cell.

In an aspect is provided a method of preventing a cytomegalovirus (CMV) infection in a subject in need thereof, the method including administering to the subject a prophylactically effective amount of an agent including UL141 or a fragment thereof. In embodiments, the agent further includes gH or a fragment thereof. In embodiments, the UL141 or fragment thereof is non-covalently bound to gH or fragment thereof. In embodiments, the UL141 or fragment thereof is covalently bound to gH or fragment thereof. In embodiments, the agent further includes UL116 or a fragment thereof. In embodiments, the UL141 or fragment thereof is non-covalently bound to UL116 or a fragment thereof. In embodiments, the UL141 or a fragment thereof is covalently bound to UL116 or fragment thereof.

In embodiments, the agent further includes gH and UL116 or fragments thereof. In embodiments, UL141, gH and UL116 or fragments thereof are non-covalently bound. In embodiments, UL141, gH and UL116 or fragments thereof are covalently bound. In embodiments, UL141 and gH or fragments thereof are non-covalently bound. In embodiments, UL141 and gH or fragments thereof are covalently bound. In embodiments, UL141 and UL116 or fragments thereof are non-covalently bound. In embodiments, UL141 and UL116 or fragments thereof are covalently bound. In embodiments, gH and UL116 or fragments thereof are non-covalently bound. In embodiments, gH and UL116 or fragments thereof are covalently bound.

For the methods provided herein, including embodiments thereof, the agent modulates an immune response in the subject. In embodiments, the agents stimulates the immune response in the subject. In embodiments, the method further includes administering an adjuvant to the subject.

In embodiments, the agent further includes a pharmaceutically acceptable excipient.

In embodiments, the CMV is human CMV (hCMV).

In an aspect is provided a method of diagnosing cytomegalovirus (CMV) infection, comprising: a) contacting a biological sample from a subject with the composition of claim 58; and b) detecting binding of the agent to the UL141/UL116/gH multimer.

In an aspect is provided a method of diagnosing cytomegalovirus (CMV) infection, including administering to a subject a composition including an agent capable of detecting an UL141 or an UL141/UL116/gH multimer. In embodiments, the composition includes an agent coupled to a diagnostic agent, wherein the agent binds a UL141/UL116/gH multimer. In embodiments, the diagnostic agent includes a metal chelator bound to a metal ion, a small molecule, an antibody or functional fragment, a radioisotope, an enzyme, an oligonucleotide, an organic or inorganic nanoparticle, a chelator, a boron compound, a photoactive agent, a dye, fluorescent or luminescent substance, an enzyme, an enhancing agent, a radioactive substance, or a chelator.

### Methods of Inhibiting Cell to Cell Spread

The compositions provided herein, including embodiments thereof, are further contemplated for inhibiting cell to cell spread of cytometagovirus (CMV). For example, inhibiting cell to cell spread may be inhibiting entry of a virus particle released from one host cell to another host cell. For example, inhibiting cell to cell spread may be inhibiting direct transfer of a virus from one host cell to another host cell. Thus, in an aspect is provided a method of inhibiting cell to cell spread of cytometagovirus, the method including administering to a subject a composition provided herein including embodiments thereof.

In embodiments, the composition includes UL141 or a fragment thereof. In embodiments, the composition includes one or more of UL141, UL116, gH, gL, gO, UL128, UL130, UL131, or fragments thereof. In embodiments, one or more of UL141, UL116, gH, gL, gO, UL128, UL130, UL131 are non-covalently bound to form a multimer (e.g. 2-mer, 3-mer, 5-mer etc.). In embodiments, one or more of UL141, UL116, gH, gL, gO, UL128, UL130, UL131 are covalently bound (e.g. by means of a covalent bond, a chemical linker, a peptide linker, bioconjugate linker etc). In embodiments, the composition includes a UL141/UL116/gH multimer. In embodiments, the composition includes a gH/gL/gO multimer. In embodiments, the composition includes a gH/gL/UL128/UL130/UL131 multimer.

In embodiments, the cell is an epithelial cell. In embodiments, the cell is an immune cell.

### Compositions

Compositions provided herein are contemplated to be useful for treating and/or preventing cytometagovirus (CMV) infections. Applicants have found that agents that modulate a UL141/UL116/gH multimer (e.g. inhibit gH, UL116 and UL141 multimerization, inhibit the function or activity of one or more of gH, UL116 or UL141, inhibit binding of one or more of gH, UL116 or UL141 to its cognate ligand etc.) are surprisingly effective for preventing or inhibiting CMV entry into a host cell, cell-to-cell spread of CMV and/or fusion of CMV with a host cell.

Thus, in an aspect is provided a vaccine composition including UL141 or a fragment thereof and a pharmaceutically acceptable excipient. In embodiments, the vaccine composition further includes gH or a fragment thereof. In embodiments, the vaccine composition further includes UL116 or a fragment thereof. In embodiments, UL141 is non-covalently bound to one or more of gH or UL116. For example, UL141 may be non-covalently bound to gH, thereby forming a multimer (e.g. a dimer or 2-mer). For example, UL141, UL116 and gH may be non-covalently bound, thereby forming a multimer (e.g. a trimer, 3-mer). In embodiments, UL141 is covalently bound to one or more of gH or UL116. For example, UL141 may be covalently bound to gH. For covalent attachment of one or more proteins, covalent conjugation methods may be used which are well known in the art and described herein. In embodiments, the vaccine composition includes a nucleic acid encoding UL141 or a fragment thereof. In embodiments, the vaccine composition includes a nucleic acid encoding gH or a fragment thereof. In embodiments, the vaccine composition includes a nucleic acid encoding UL116 or a fragment thereof. In embodiments, the nucleic acid is DNA. In embodiments, the nucleic acid is RNA. In embodiments, the nucleic acid is mRNA.

Compositions provided herein including embodiments thereof may be used to diagnose CMV infection. In instances, the compositions may be used to detect CMV in a subject infected with or at risk of being infected with CMV. In an aspect is provided a composition including an agent coupled to a diagnostic agent, wherein the agent binds a UL141/UL116/gH multimer. In embodiments, the diagnostic agent includes a metal chelator bound to a metal ion, a small molecule, an antibody or functional fragment, a radioisotope, an enzyme, an oligonucleotide, an organic or inorganic nanoparticle, a chelator, a boron compound, a photoactive agent, a dye, fluorescent or luminescent substance, an enzyme, an enhancing agent, a radioactive substance, or a chelator.

### Nucleic Acids and Vectors

The agents provided herein, including embodiments thereof, may be delivered to the cell in a variety of methods known in the art. In embodiments, the agents comprise one or more of the proteins and or nucleic acids described herein, including embodiments thereof. Thus, the agents may be expressed transiently, bypassing the necessity of viral delivery methods. The agents may be encoded on RNA or DNA delivered to cells as a modified or unmodified RNA or plasmid DNA. In embodiments, the RNA is mRNA. The RNA or DNA encoding the agents may be delivered by transfection, lipid nanoparticle, virus like particle (VLP) or virus. In theory, the agents may also be directly delivered via transfection or lipid nanoparticle or VLP.

The agents described herein, including embodiments and aspects thereof, may be provided as a nucleic acid sequence that encodes for a protein provided herein including embodiments thereof. Thus, in an aspect is provided a nucleic acid sequence encoding an agent described herein, including embodiments and aspects thereof. In an aspect is provided a nucleic acid sequence encoding an agent described herein, including embodiments and aspects thereof. In aspects, the nucleic acid sequence encodes for a protein described herein, including proteins having amino acid sequences with certain percent sequence identities described herein. In aspects, the nucleic acid sequence encodes for a protein described herein, including proteins having amino acid sequences with certain percent sequence identities or homology described herein.

In embodiments, the agent is a nucleic acid encoding UL141 or functional portion thereof.

In embodiments, the methods provided herein further include a nucleic acid encoding gH, gL, gO, UL116, UL128, UL130, UL131, or a functional portion thereof. In embodiments, the nucleic acid encodes gH, or a functional portion thereof. In embodiments, the nucleic acid encodes gL, or a functional portion thereof. In embodiments, the nucleic acid encodes gO, or a functional portion thereof. In embodiments, the nucleic acid encodes UL116, or a functional portion thereof. In embodiments, the nucleic acid encodes UL128, or a functional portion thereof. In embodiments, the nucleic acid encodes UL130, or a functional portion thereof. In embodiments, the nucleic acid encodes UL131, or a functional portion thereof.

In embodiments, the agent is a nucleic acid encoding a receptor that binds UL141, UL141/UL116/gH, or a multimer including UL141.

In embodiments, the agent is a nucleic acid encoding TRAIL receptor, CD155, CD1121, or a functional portion thereof. In embodiments, the agent is a nucleic acid encoding TRAIL receptor, or a functional portion thereof. In embodiments, the agent is a nucleic acid encoding CD155, or a functional portion thereof. In embodiments, the agent is a nucleic acid encoding CD1121, or a functional portion thereof.

In embodiments, the TRAIL receptor is TRAIL receptor 1, TRAIL receptor 2, TRAIL receptor 3, or TRAIL receptor 4. In embodiments, the TRAIL receptor is TRAIL receptor 1. In embodiments, the TRAIL receptor is TRAIL receptor 2. In embodiments, the TRAIL receptor is TRAIL receptor 3. In embodiments, the TRAIL receptor is TRAIL receptor 4.

For the methods provided herein, in embodiments, the agent is a nucleic acid encoding a modified receptor protein described herein, including embodiments thereof. In embodiments, the modified receptor protein includes an Fc domain.

In embodiments, the agent is a nucleic acid encoding an antibody or antigen binding fragment. In embodiments, the nucleic acid encodes an antibody. In embodiments, the nucleic acid encodes an antigen binding fragment. In embodiments, the antibody or antigen binding fragment is a human antibody, a monoclonal antibody, a polyclonal antibody, a single chain antibody, Fab, Fab′, F(ab′)2, Fv or scFv. In embodiments, the antibody or antigen binding fragment is a human antibody. In embodiments, the antibody or antigen binding fragment is a monoclonal antibody. In embodiments, the antibody or antigen binding fragment is a polyclonal antibody. In embodiments, the antibody or antigen binding fragment is a single chain antibody. In embodiments, the antibody or antigen binding fragment is a Fab. In embodiments, the antibody or antigen binding fragment is a Fab′. In embodiments, the antibody or antigen binding fragment is a F(ab′)2. In embodiments, the antibody or antigen binding fragment is a Fv. In embodiments, the antibody or antigen binding fragment is a scFv.

In embodiments, the agent is a nucleic acid encoding an antibody or an antigen binding fragment thereof, wherein the antibody or antigen binding fragment thereof binds a multimer comprising UL141.

In embodiments, the agent is a nucleic acid encoding a multispecific antibody, wherein the multispecific antibody recognizes at least one epitope of UL141. In embodiments, the agent is a nucleic acid encoding a bispecific or a trispecific antibody and wherein the antibody recognizes at least one epitope of UL141. In embodiments, the antibody is a bispecific antibody wherein the antibody recognizes at least one epitope of UL141. In embodiments, the antibody is a trispecific antibody wherein the antibody recognizes at least one epitope of UL141.

For the methods provided herein, in embodiments, the nucleic acid further includes a vector. In embodiments, the vector is a viral vector. In embodiments, the viral vector further includes a recombinant virus.

In an aspect is provided a composition including the nucleic acid of SEQ ID NO:11, 13, 19, 22, 25, 28, 31, or 33, or a combination thereof.

In an aspect is provided a composition comprising the nucleic acids provided herein, or the encoded peptides.

### EMBODIMENTS

P Embodiment 1. A method of treating or preventing a cytomegalovirus (CMV) infection in a subject in need thereof, the method comprising administering to said subject an effective amount of an agent that modulates a UL141/UL116/gH multimer.

P Embodiment 2. The method of P embodiment 1, wherein said agent modulates (a) binding of CMV to a host cell, (b) fusion of CMV with a host cell, (c) cell-to-cell spread of CMV or (d) multimerization of CMV proteins gH, UL116 and UL141.

P Embodiment 3. The method of P embodiment 1, wherein said agent (a) inhibits formation of a UL141/UL116/gH multimer (b) inhibits binding of UL141 to UL116/gH, or (c) inhibits binding of said multimer to a ligand thereof.

P Embodiment 4. The method of P embodiment 1, wherein said agent comprises UL141 or a functional portion thereof.

P Embodiment 5. The method of P embodiment 1, wherein said agent comprises one or more of UL141, UL116, gH, gL, gO, UL128, UL130, UL131, or a functional portions thereof.

P Embodiment 6. The method of P embodiment 5, wherein said agent comprises the UL141/UL116/gH multimer.

P Embodiment 7. The method of P embodiment 5, wherein said agent comprises a gH/gL/gO multimer.

P Embodiment 8. The method of P embodiment 5, wherein said agent comprises a gH/gL/UL128/UL130/UL131 multimer.

P Embodiment 9. The method of P embodiment 5, wherein said agent comprises an multimer comprising two or more of UL141, UL116, gH, gL, gO, UL128, UL130, UL131, or fragment thereof.

P Embodiment 10. The method of P embodiment 1, wherein said agent is a TRAIL receptor, CD155, or CD112 or fragment thereof.

P Embodiment 11. The method of P embodiment 10, wherein said TRAIL receptor is TRAIL receptor 1, TRAIL receptor 2, TRAIL receptor 3, TRAIL receptor 4, Osteoprotegrin (OPG) or fragment thereof.

P Embodiment 12. The method of any of P embodiments 5-11, wherein said agent is a modified receptor protein.

P Embodiment 13. The method of P embodiment 12, wherein said modified receptor protein comprises an Fc domain.

P Embodiment 14. The method of P embodiment 1, wherein said agent is an antibody or antigen binding fragment.

P Embodiment 15. The method of P embodiment 14, wherein said antibody or antigen binding fragment is a human antibody, a monoclonal antibody, a polyclonal antibody, a single chain antibody, Fab, Fab′, F(ab′)2, Fv or scFv.

P Embodiment 16. The method of P embodiment 14, wherein said antibody is an anti-UL141, anti-TRAIL receptor, anti-CD155, or anti-CD112 antibody.

P Embodiment 17. The method of P embodiment 14, wherein said antibody or antigen binding fragment recognizes a multimer comprising UL141.

P Embodiment 18. The method of P embodiment 14, wherein said agent is a multispecific antibody that recognizes at least one epitope of UL141.

P Embodiment 19. The method of P embodiment 18, wherein said multispecific antibody is a bispecific antibody.

P Embodiment 20. The method of P embodiment 18, wherein said multispecific antibody is a trispecific antibody.

P Embodiment 21. The method of P embodiment 1, wherein said agent is a nucleic acid encoding UL141 or fragment thereof.

P Embodiment 22. The method of P embodiment 21, further comprising a nucleic acid encoding gH, gL, gO, UL116, UL128, UL130, UL131, or a fragment thereof.

P Embodiment 23. The method of P embodiment 1, wherein said agent is a nucleic acid encoding a TRAIL receptor, CD155, CD1121, or a fragment thereof.

P Embodiment 24. The method of P embodiment 23, wherein the TRAIL receptor is TRAIL receptor 1, TRAIL receptor 2, TRAIL receptor 3, TRAIL receptor 4 or OPG.

P Embodiment 25. The method of P embodiment 1, wherein said agent is a nucleic acid encoding a modified receptor protein.

P Embodiment 26. The method of P embodiment 25, wherein said modified receptor protein comprises an Fc domain.

P Embodiment 27. The method of P embodiment 1, wherein said agent is a nucleic acid encoding an antibody or antigen binding fragment.

P Embodiment 28. The method of P embodiment 27, wherein said antibody or antigen binding fragment is a human antibody, a monoclonal antibody, a polyclonal antibody, a single chain antibody, Fab, Fab′, F(ab′)2, Fv or scFv.

P Embodiment 29. The method of P embodiment 27, wherein said antibody or antigen binding fragment recognizes a multimer comprising UL141.

P Embodiment 30. The method of P embodiment 27, wherein said antibody is a multispecific antibody that recognizes at least one epitope of UL141.

P Embodiment 31. The method of P embodiment 30, wherein said multispecific antibody is a bispecific antibody.

P Embodiment 32. The method of P embodiment 30, wherein said multispecific antibody is a trispecific antibody.

P Embodiment 33. The method of any of P embodiments 21-32, wherein said nucleic acid further comprises a vector.

P Embodiment 34. The method of P embodiment 33, wherein said vector is a viral vector.

P Embodiment 35. The method of P embodiment 34, wherein said viral vector further comprises a recombinant virus.

P Embodiment 36. The method of P embodiment 1, wherein said agent comprises a small molecule, a peptide mimetic, an aptamer, or an inhibitory nucleic acid.

P Embodiment 37. The method of any of P embodiments 1-13 or P embodiments 21-25, wherein said agent promotes an immune response in said subject.

P Embodiment 38. The method of P embodiment 37, said method further comprising administering an adjuvant to said subject.

P Embodiment 39. The method of any of P embodiments 1-38, wherein said agent further comprises a pharmaceutically acceptable excipient.

P Embodiment 40. The method of any of P embodiments 1-39, wherein CMV is human CMV (hCMV).

P Embodiment 41. A method for screening agents that inhibit cytomegalovirus (CMV) host cell entry, fusion of CMV with a host cell, or cell-to-cell spread of CMV, comprising administering a test agent to a CMV-receptive cell, administering CMV to said CMV-receptive cell, and determining whether said agent modulates a UL141/UL116/gH multimer.

P Embodiment 42. A composition comprising an agent coupled to a diagnostic agent, wherein said agent binds a UL141/UL116/gH multimer.

P Embodiment 43. The composition of P embodiment 42, wherein the diagnostic agent comprises a metal chelator bound to a metal ion, a small molecule, an antibody or functional fragment, a radioisotope, an enzyme, an oligonucleotide, an organic or inorganic nanoparticle, a chelator, a boron compound, a photoactive agent, a dye, fluorescent or luminescent substance, an enzyme, an enhancing agent, a radioactive substance, or a chelator.

P Embodiment 44. A method of diagnosing cytomegalovirus (CMV) infection in a subject, comprising: a) contacting a biological sample from said subject with the composition of embodiment 58; and b) detecting binding of said agent to the UL141/UL116/gH multimer.

P Embodiment 45. A composition comprising nucleic acids encoding proteins having at least 80% sequence identity to the amino acid sequences of SEQ ID NO:15, 16, 17, 20, 22, 23, 26, 28, or 31.

P Embodiment 46. A composition comprising one or more nucleic acids provided herein, or the encoded polypeptides.

### Embodiments

Embodiment 1. A method of treating or preventing a cytomegalovirus (CMV) infection in a subject in need thereof, the method comprising administering to said subject a therapeutically or prophylactically effective amount of an agent that modulates a UL141/UL116/gH multimer.

Embodiment 2. The method of embodiment 1, wherein said agent modulates (a) binding of CMV to a host cell, (b) fusion of CMV with a host cell, (c) cell-to-cell spread of CMV or (d) multimerization of CMV proteins gH, UL116 and UL141.

Embodiment 3. The method of embodiment 1, wherein said agent (a) inhibits formation of the UL141/UL116/gH multimer (b) inhibits binding of UL141 to UL116/gH, or (c) inhibits binding of said multimer to a ligand thereof.

Embodiment 4. The method of any one of embodiments 1-3, wherein said agent comprises UL141 or a fragment thereof.

Embodiment 5. The method of any one of embodiments 1-4, wherein said agent comprises one or more of UL141, UL116, gH, gL, gO, UL128, UL130, UL131, or a fragment thereof.

Embodiment 6. The method of any one of embodiments 1-5, wherein said agent comprises the UL141/UL116/gH multimer.

Embodiment 7. The method of any one of embodiments 1-5, wherein said agent comprises a gH/gL/gO multimer.

Embodiment 8. The method of any one of embodiments 1-5, wherein said agent comprises a gH/gL/UL128/UL130/UL131 multimer.

Embodiment 9. The method of any one of embodiments 1-5, wherein said agent comprises an multimer comprising two or more of UL141, UL116, gH, gL, gO, UL128, UL130, UL131, or fragments thereof.

Embodiment 10. The method of any one of embodiments 1-3, wherein said agent is a TRAIL receptor, CD155, CD112 or a fragment thereof.

Embodiment 11. The method of embodiment 10, wherein said TRAIL receptor is TRAIL receptor 1, TRAIL receptor 2, TRAIL receptor 3, TRAIL receptor 4, Osteoprotegrin (OPG) or a fragment thereof.

Embodiment 12. The method of any one of embodiments 1-3, wherein said agent is a modified receptor protein.

Embodiment 13. The method of embodiment 12, wherein said modified receptor protein comprises an Fc domain.

Embodiment 14. The method of any one of embodiments 1-3, wherein said agent is an antibody or antigen binding fragment thereof.

Embodiment 15. The method of embodiment 14, wherein said antibody or antigen binding fragment is a human antibody, a monoclonal antibody, a polyclonal antibody, a single chain antibody, Fab, Fab′, F(ab′)2, Fv or scFv.

Embodiment 16. The method of embodiment 14 or 15, wherein said antibody is an anti-UL141, anti-TRAIL receptor, anti-CD155, or anti-CD112 antibody.

Embodiment 17. The method of embodiment 14 or 15, wherein said antibody or antigen binding fragment thereof recognizes a multimer comprising UL141.

Embodiment 18. The method of embodiment 14, wherein said agent is a multispecific antibody that recognizes at least one epitope of UL141.

Embodiment 19. The method of embodiment 18, wherein said multispecific antibody is a bispecific antibody.

Embodiment 20. The method of embodiment 18 or 19, wherein said multispecific antibody is a trispecific antibody.

Embodiment 21. The method of any one of embodiments 1-3, wherein said agent comprises a nucleic acid encoding UL141 or fragment thereof.

Embodiment 22. The method of embodiment 21, wherein said agent further comprises a nucleic acid encoding gH, gL, gO, UL116, UL128, UL130, UL131, or a fragment thereof.

Embodiment 23. The method of embodiment 1, wherein said agent comprises a nucleic acid encoding a TRAIL receptor, CD155, CD1121, or a fragment thereof.

Embodiment 24. The method of embodiment 23, wherein the TRAIL receptor is TRAIL receptor 1, TRAIL receptor 2, TRAIL receptor 3, TRAIL receptor 4 or OPG.

Embodiment 25. The method of embodiment 1, wherein said agent comprises a nucleic acid encoding a modified receptor protein.

Embodiment 26. The method of embodiment 25, wherein said modified receptor protein comprises an Fc domain.

Embodiment 27. The method of embodiment 1, wherein said agent comprises a nucleic acid encoding an antibody or antigen binding fragment.

Embodiment 28. The method of embodiment 27, wherein said antibody or antigen binding fragment is a human antibody, a monoclonal antibody, a polyclonal antibody, a single chain antibody, Fab, Fab′, F(ab′)2, Fv or scFv.

Embodiment 29. The method of embodiment 27 or 28, wherein said antibody or antigen binding fragment recognizes a multimer comprising UL141.

Embodiment 30. The method of embodiment 27, wherein said antibody is a multispecific antibody that recognizes at least one epitope of UL141.

Embodiment 31. The method of embodiment 30, wherein said multispecific antibody is a bispecific antibody.

Embodiment 32. The method of embodiment 30, wherein said multispecific antibody is a trispecific antibody.

Embodiment 33. The method of any of embodiments 21-32, wherein said nucleic acid further comprises a vector.

Embodiment 34. The method of embodiment 33, wherein said vector is a viral vector.

Embodiment 35. The method of embodiment 34, wherein said viral vector further comprises a recombinant virus.

Embodiment 36. The method of embodiment 1, wherein said agent comprises a small molecule, a peptide mimetic, an aptamer, or an inhibitory nucleic acid.

Embodiment 37. The method of any of embodiments 1-13 or 21-25, wherein said agent promotes an immune response in said subject.

Embodiment 38. The method of embodiment 37, said method further comprising administering an adjuvant to said subject.

Embodiment 39. The method of any of embodiments 1-38, wherein said agent further comprises a pharmaceutically acceptable excipient.

Embodiment 40. The method of any of embodiments 1-39, wherein CMV is human CMV (hCMV).

Embodiment 41. A method of preventing a cytomegalovirus (CMV) infection in a subject in need thereof, the method comprising administering to said subject a prophylactically effective amount of an agent comprising UL141 or a fragment thereof.

Embodiment 42. The method of embodiment 41, wherein said agent further comprises gH or a fragment thereof.

Embodiment 43. The method of embodiment 42, wherein said UL141 s non-covalently bound to gH.

Embodiment 44. The method of embodiment 42, wherein said UL141 is covalently bound to gH.

Embodiment 45. The method of any one of embodiments 41-43, wherein said agent further comprises UL116.

Embodiment 46. The method of embodiment 45, wherein UL141 thereof is non-covalently bound to UL116.

Embodiment 47. The method of embodiment 45, wherein UL141 is covalently bound to UL116.

Embodiment 48. The method of embodiment 45, further comprising gH and UL116 or fragments thereof.

Embodiment 49. The method of embodiment 48, wherein said UL141, gH and UL116 are non-covalently bound.

Embodiment 50. The method of embodiment 48, wherein said UL141, gH and UL116 are covalently bound.

Embodiment 51. A vaccine composition comprising UL141 or a fragment thereof and a pharmaceutically acceptable excipient.

Embodiment 52. The vaccine composition of embodiment 51, further comprising gH or a fragment thereof.

Embodiment 53. The vaccine composition of embodiment 51 or 52, further comprising UL116 or a fragment thereof.

Embodiment 54. A method for screening agents that inhibit cytomegalovirus (CMV) host cell entry, fusion of CMV with a host cell, or cell-to-cell spread of CMV, comprising administering a test agent to a CMV-receptive cell, administering CMV to said CMV-receptive cell, and determining whether said agent modulates a UL141/UL116/gH multimer.

Embodiment 55. A composition comprising an agent coupled to a diagnostic agent, wherein said agent binds a UL141/UL116/gH multimer.

Embodiment 56. The composition of embodiment 55, wherein the diagnostic agent comprises a metal chelator bound to a metal ion, a small molecule, an antibody or functional fragment, a radioisotope, an enzyme, an oligonucleotide, an organic or inorganic nanoparticle, a chelator, a boron compound, a photoactive agent, a dye, fluorescent or luminescent substance, an enzyme, an enhancing agent, a radioactive substance, or a chelator.

Embodiment 57. A method of diagnosing cytomegalovirus (CMV) infection in a subject, comprising: a) contacting a biological sample from said subject with the composition of embodiment 55 or 56; and b) detecting binding of said agent to the UL141/UL116/gH multimer.

Embodiment 58. A composition comprising a nucleic acid encoding a protein having at least 80% sequence identity to the amino acid sequence of SEQ ID NO:15, 16, 17, 20, 22, 23, 26, 28, or 31.

Embodiment 59. A composition comprising one or more nucleic acids provided herein, or proteins encoded by said nucleic acids.

### Examples

### Example 1: Introduction to Exemplary Studies

It is a long-held dogma in the herpesvirus field that: (i) gL-null viruses are incapable of cell entry, and (ii) that gH does not mature beyond the ER to become incorporated into virions without its binding partner, gL. The fact that a gL-null mutant of rhesus rhadinovirus (RRV, a gamma-2 herpesvirus) can infect B-cells in vivo²⁷ indicates point (i) is not a strict rule. In turn, point (ii) is directly disputed by recent work showing that HCMV virions contain a gL-free gH/UL116 complex, and that gH export from the ER can occur with UL116 in lieu of gL¹⁹. Data described herein show that this gH/UL116 complex has a third component, UL141. Previous work dissecting the role of UL141 role as an immunoevasin showed it can bind directly to the TRAIL-DRs and CD155 with high affinity^{21,28,29}. The studies described herein (i) define a new HCMV gH complex, (ii) establish a potential mechanistic link between an immunoevasin and a core component of the herpesvirus entry machinery and (iii) represent a new target for HCMV vaccine approaches.

Similar to components of another gH/gL complex, pentamer (e.g. gH/gL/UL128/UL130/UL131), UL141 is often spontaneously disrupted during tissue culture passage. UL141 restricts the cell surface expression of TRAIL death receptors (TRAIL-DRs)²¹, CD155/PVR²² and Nectin-2/CD112²³ by sequestering them within the ER. This inhibits natural killer (NK) cell lysis of infected cells which express UL141^{21,22}. Paralleling these results, m166 in mouse CMV (MCMV) similarly restricts expression of TRAIL-DR, and MCMV mutants disrupted for m166 are crippled for viral replication in both the early and persistent phases due to the inability to block TRAIL-mediated killing by innate lymphoid type I cells (ILC1)^{24,25}. These roles for UL141 and m166 in dampening TRAIL-dependent innate immune control of CMV replication are consistent with their appreciated functions as immunoevasins. However, data provided herein shows that UL141 associates with gH/UL116, and its incorporation into the virion envelope was unexpected. Without wishing to be bound by scientific theory, the UL141-containing HCMV glycoprotein complex may play dual roles in immune evasion and entry.

### Example 2: UL141 Contributions to HCMV Cell Tropism

Without wishing to be bound by scientific theory, it was thought that UL141 is the receptor binding moiety of a virion-incorporated UL141/gH/UL116 complex that engages cellular receptors and mediates viral entry. Unlike gH and UL116, which are stably expressed by laboratory adapted HCMV strains, UL141 disruption has been observed in HCMV strains that have been serially propagated in cultured fibroblasts. These same lab-adapted viruses also show genetic disruptions in components of the pentamer (e.g. gH/gL/UL128/UL130/UL131), rendering them largely incapable of infecting non-fibroblast cell types. Thus, studies are completed to determine receptor requirements and cell types involved in UL141-dependent HCMV entry. The studies further resolve which UL141 binding partner(s) can enhance pentamer-independent spread in ARPE-19 epithelial cells. In addition, whether UL141 utilizes similar pathways to enter other human cell types is established. The study also determines whether ectopic expression of known UL141 binding partners enhances cell permissivity for HCMV infection in a UL141-dependent manner.

Identify Receptor Requirements and Cell Types that Support UL141-Enhanced Viral Entry Experimental Approach

The initial focus was on ARPE-19 cells, as data indicated that UL141 greatly enhances infection and/or spread in this epithelial cell line. Since UL141 is established to directly bind TRAIL-DRs²¹ and CD155 (PVR)²², and to be required during infection for intracellular retention of Nectin-2²³, flow cytometry was used to profile the surface expression patterns of these candidate UL141-entry receptors. The expression profile of receptors known to facilitate HCMV entry via the trimeric or pentameric gH/gL complexes is determined (PDGFRα^{8-19,39}, NRP2¹¹, OR14I1¹² and CD147¹³). This is performed in both RPE-1 and ARPE-19, since these two retinal pigment epithelial lines may differ in their permissivity for UL141-restored, pentamer-null HCMV. In parallel, an established siRNA reverse-transfection approach^{40,41} is utilized to test if knocking down these expressed molecules impacts UL141-dependent entry. Western blotting and flow cytometry are applied to confirm knockdown levels. These coordinated studies reveal the phenotypic profile and receptor dependency for the UL141-enhanced, pentamer-independent mode of viral infectivity of ARPE-19.

Mutant viruses for studies were generated using ‘en passant’ BAC mutagenesis^{45,46}, as previously described^{32,34,40,47-49}. For instance, a variant of HCMV strain AD169rv, which ordinarily lacks UL141, was modified with the UL141 coding sequence from strain TR3.1.⁴¹ The UL141-restored virus is referred to as Δ128L_r141. Moreover, GFP-expressing versions of Δ128_r141 and AD169rv were made.

Infectivity Assays^{32,34,40,47-49}

Stocks of viruses are quantified for infectious titer on fibroblasts by TCID50 assay. Viruses being compared for infectivity (on a given cell type or following an siRNA treatment) are diluted to 10⁵ TCID50/mL, and a 10-fold serial dilution series is applied to sub-confluent cells in 96-well plates. Regardless of the cell type(s) being evaluated, a parallel titration is always carried out on fully permissive fibroblasts for comparison. qPCR-based titer assays are also performed in ARPE-19 and any other cell types where UL141-dependent entry/tropism is seen, allowing us to calculate cell-type specific infectivity differences between AD169rv and Δ128L_r141 on the basis of # of TCID50 units (on a given cell type) per 10E5 viral genomes³². For standard titrations, 0.1 mL of each dilution (starting at 10E5 fibroblast TCID50 units/mL, or at 10E6 viral genomes/mL) is applied to each of 8 replicate wells (or 2 replicate wells when screening siRNA treatments). Plates are scored at 24 h postinfection (hpi) by staining for IE1 or visualizing GFP. Detection of one or more GFP+/IE1+ cells defines a well as “infected.” However, for cell types that are permissive for HCMV replication, a duplicate titration plate is incubated until 7 days post-infection (dpi), and in this case wells are scored as “infected” only if one or more IE1+ plaques are found. For GFP viruses, the plate scored at 24 hpi is simply returned to the incubator until 7 dpi. Employing both 24 hpi (entry) and 7 dpi (plaque formation) criteria enables us to determine whether UL141 provides an advantage: (i) at the initial cell entry step and/or (ii) in secondary spread between cells. Notably, the pentamer enhances HCMV infection at both of these steps^{32,38}. For ARPE-19, and in any other cell types in which UL141-dependent infectivity is seen, triplicate biological replicates are performed comparing TCID50 values on permissive fibroblasts versus the ‘restrictive’ cell type (i.e., where pentamer null virus lacking UL141 fails to efficiently infect). The infectivity ratio between UL141+ versus UL141-null virus on the two cell types is evaluated by an unpaired T-test, or by one- or two-way ANOVA, with Tukey's post-test, depending on the number of groups and variables being compared (e.g., # of virus genotypes, # of cell types). This methodology contributes rigor and reproducibility to these studies.

Investigation of Whether Ectopic Expression of UL141 Binding Partners Promote Infection in the Absence of the Pentamer

The same viruses described above are used to infect ARPE-19 cells stably transduced with “tet-on” lentivirus vectors expressing known cognate ligands of UL141 (e.g. TRAIL-DRs, CD155 and CD112). Infection is done 24 h after inducing transgene expression using doxycycline (dox). ARPE-19 were chosen because efficient infection and robust multi-cycle replication in this cell type (i) requires the pentamer^{11,12,38,50}, and (ii) a UL141+AD169rv strain lacking the pentamer infects them poorly relative to RPE-1. Consequently, use of ARPE-19 provides the opportunity to test whether ectopic expression of candidate UL141/gH/UL116 receptors can enhance UL141-dependent entry. Although it is expected that ARPE-19 expresses endogenous levels of at least one or more UL141 binding partners, inducible ectopic overexpression is used since they may be upregulated in the context of in vivo infection. Therefore, ARPE-19 may serve as a surrogate model to test whether entry efficiency can vary based on receptor expression levels. The quantitative infectivity assays described herein is employed in these experiments. As additional controls, TCID50 is measured in cells not treated with dox, and in empty-vector transduced ARPE-19. When UL141 is found to enhance infection, soluble UL141/gH/UL116 complex or TRAIL-R2:Fc fusion protein²¹ are tested for whether they neutralizes this. Positive results are repeated in triplicate.

‘Tet-on’ lentivirus vectors in ARPE-19^{33,34} have previously been shown to be successful, and large amounts of purified TRAIL-R2:Fc²¹ have already been generated, including versions mutated at Asp265 in the Fc domain to abolish Fc-receptor binding,^{51,52}. It is anticipated that siRNA silencing of one or more of the known UL141 binding targets in ARPE-19 prevents UL141-dependent, pentamer-independent entry. If TRAIL-R2:Fc blocks UL141-dependent viral entry, this may indicate TRAIL-R2 is the sole specific entry receptor. However, it has been observed that TRAIL-R2 binding site on UL141 overlaps with TRAIL-R1 and CD155, albeit to varying degrees, indicating results obtained in blockade experiments need to be considered in this context. It is anticipated that likely the same UL141 binding partner(s) found to be necessary for UL141-dependent infection using siRNA knockdown promotes entry into ARPE-19 cells when expressed ectopically, as they are both epithelial cell lines. It is expected that UL141+ pentamer-null virus may not be able to spread efficiently from cell-to-cell simply by restoring UL141, as pentamer is known to play a key role in this⁵³.

Results indicate it is unlikely that UL141 binding partners expressed by ARPE-19 that promote UL141-dependent HCMV entry into this cell type are unidentifiable. Whether this receptor(s) operates in other epithelial cell lines or other cell types is under investigation. Since TRAIL-DRs can show cytoxicity when over-expressed in some cell types, dox-inducible ARPE-19 cells expressing TRAIL-R2 either deleted for the death domain or with the TRAIL-R2 ectodomain GPI-anchored to the cell surface are produced, as previously described&. Notably, if the GPI-anchored ectodomain of TRAIL-R2 supports UL141-dependent entry, this demonstrates that cytoplasmic signaling is not strictly required for viral entry, as seen for trimer mediated entry via PDGFRα^{9,10}.

Because UL141/gH/UL116 may not be able to promote the broad-spectrum cell infectivity that the pentamer does, other cell types are tested. The tropism comparisons and entry dependency studies outlined above may also be completed with pentamer-null TR3.1 that is either disrupted or intact for UL141, and this is a useful method to validate results obtained with the AD169 viruses.

It has been shown that reverse transfection of siRNA achieves excellent knockdown in human fibroblasts^{40,41}, but the Neon™ capillary electroporation system can be tried as an alternative to transfect siRNA if epithelial cell lines⁵⁴ are not ideal. If siRNA and ectopic expression approaches directed at known UL141 binding targets fail to identify a specific receptor, a whole genome CRISPR screen using the Brunello library⁵⁵ (from Addgene) is an alternate approach.

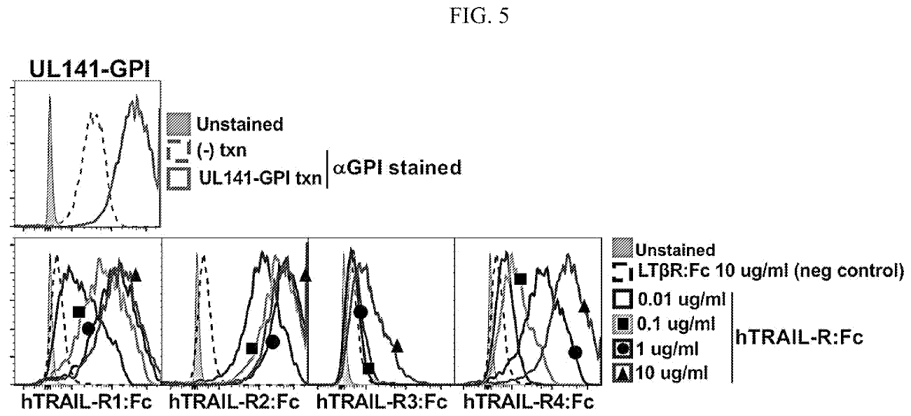

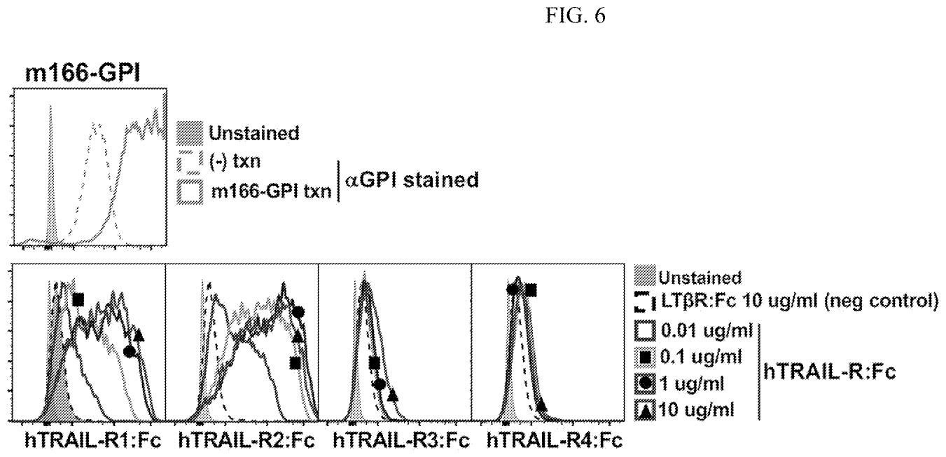

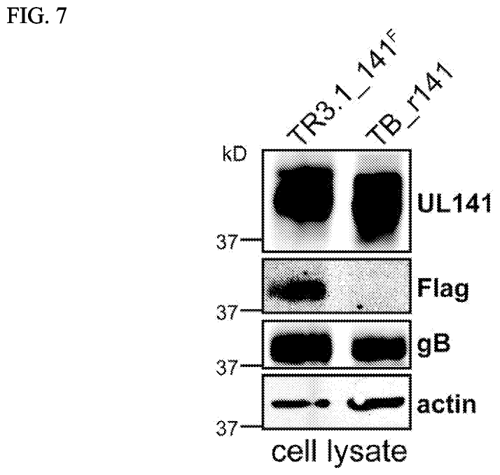

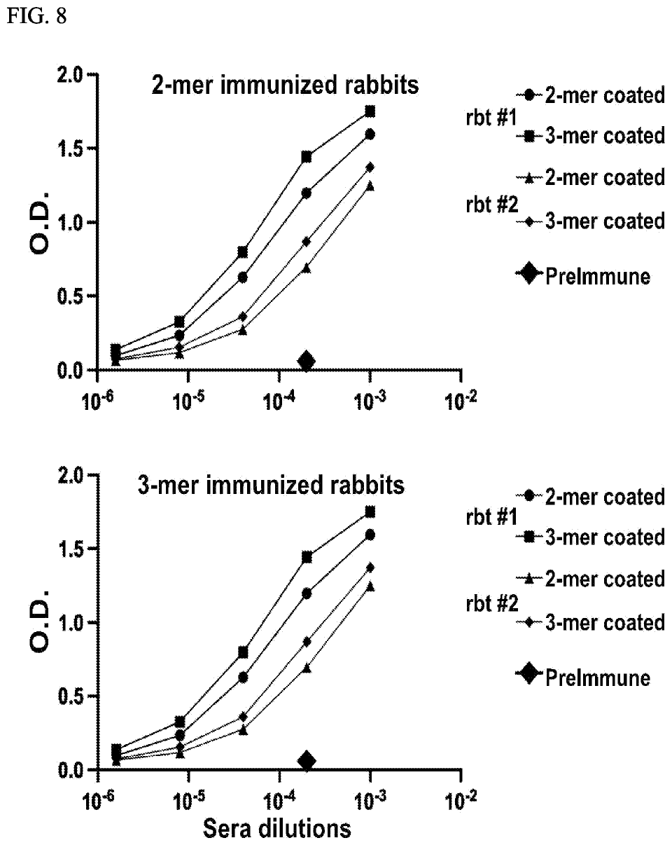

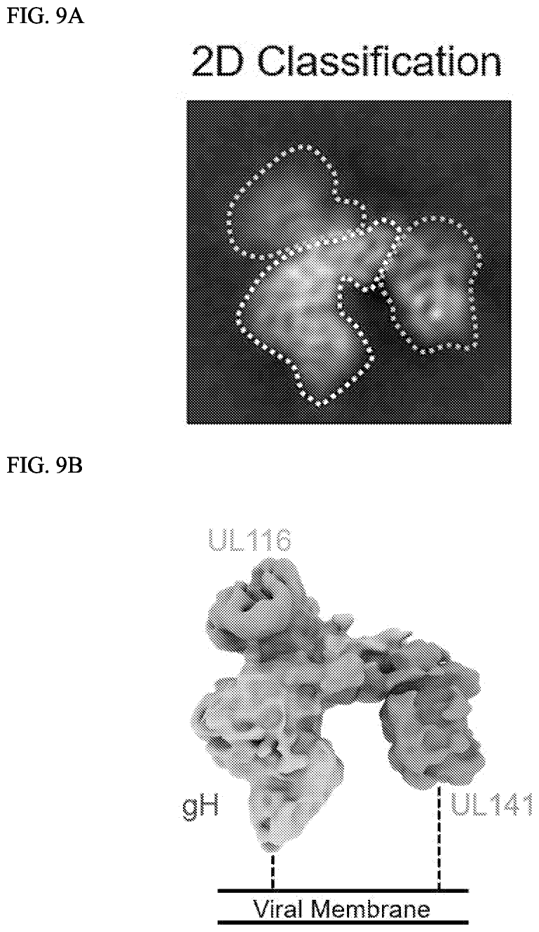

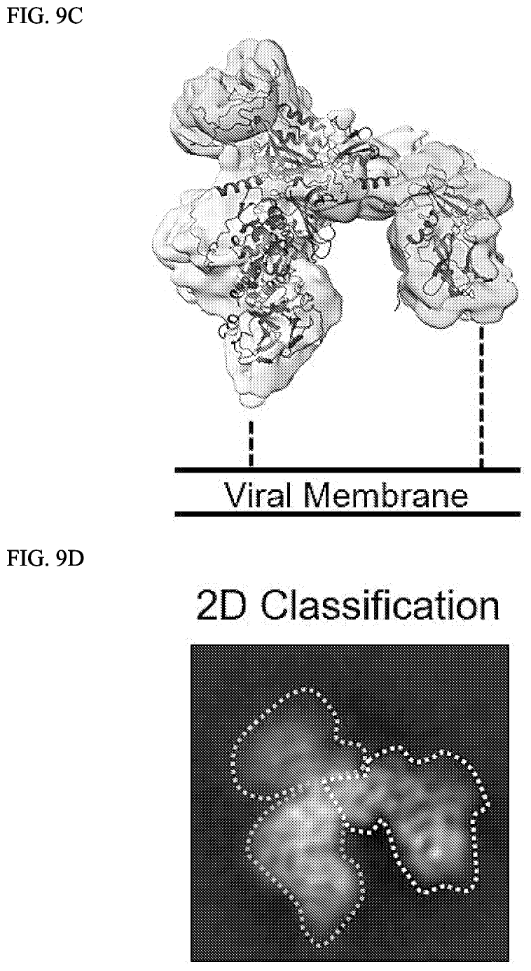

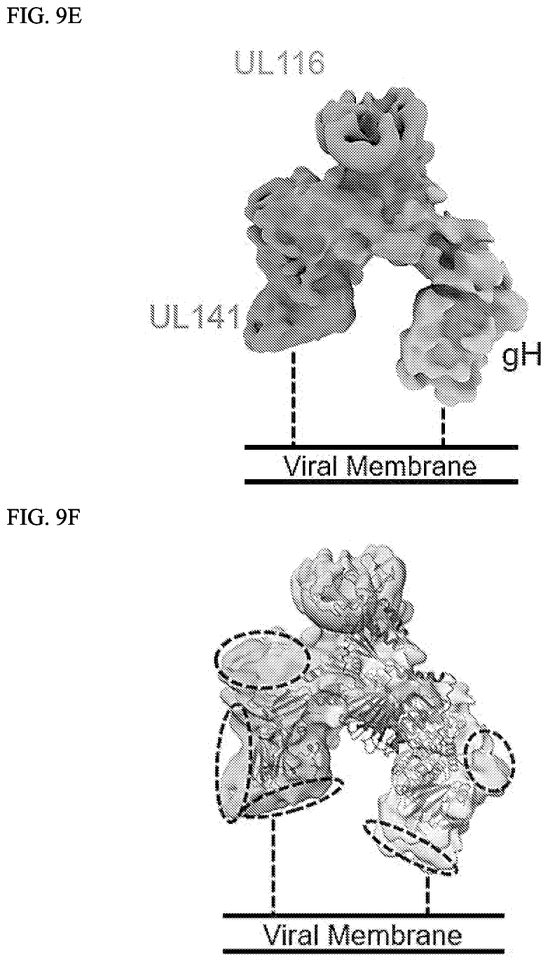

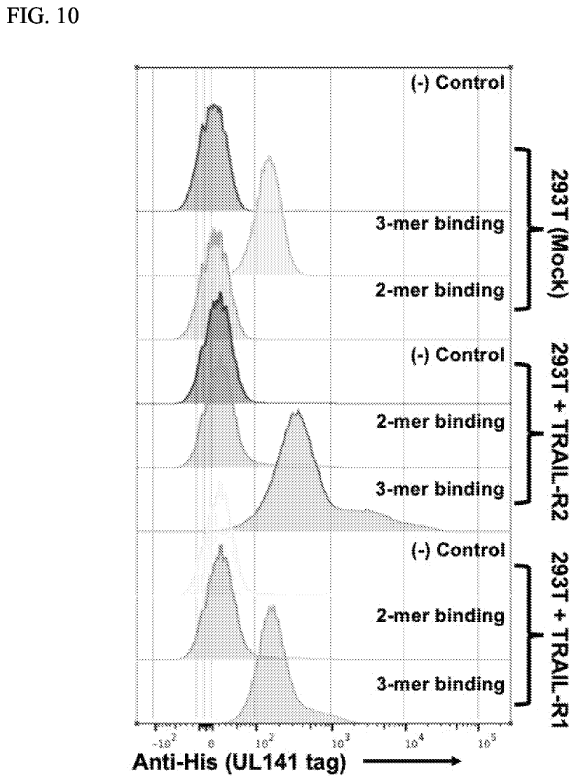

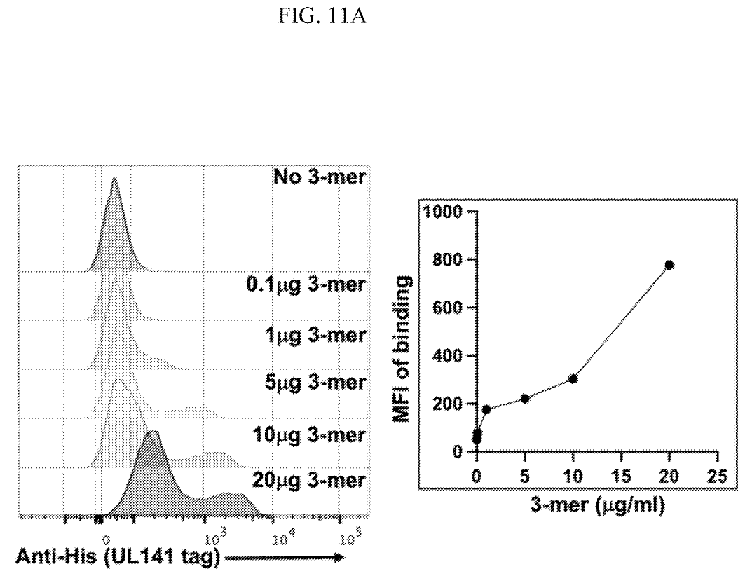

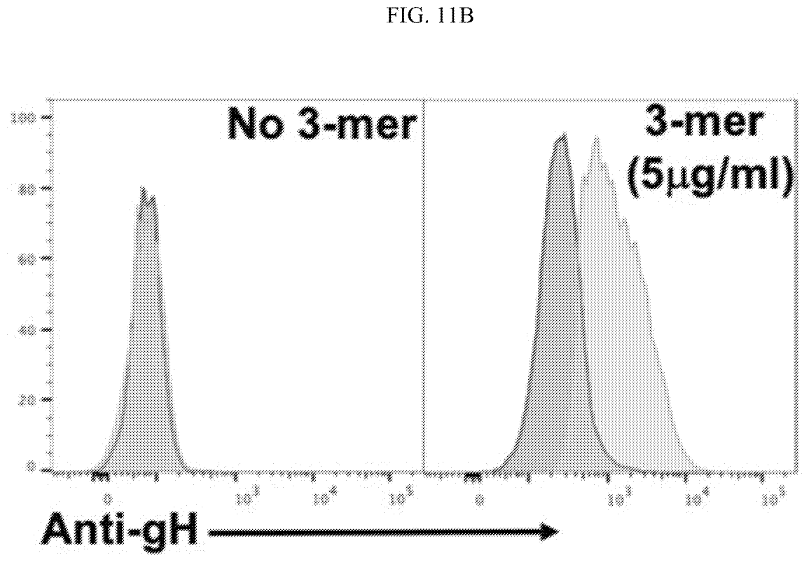

In addition to UL141 binding directly to TRAIL-R1 and -R2, it also binds to TRAIL-R4, as illustrated in FIG. 5. Although TRAIL-R4 lacks a cytoplasmic death domain, a role for TRAIL-R4 as a potential UL141-dependent entry receptor is investigated in the above-described experiments.

### Example 3: Investigation of Whether UL141/gH/UL116 Trigger Membrane Fusion when Co-Expressed with gB

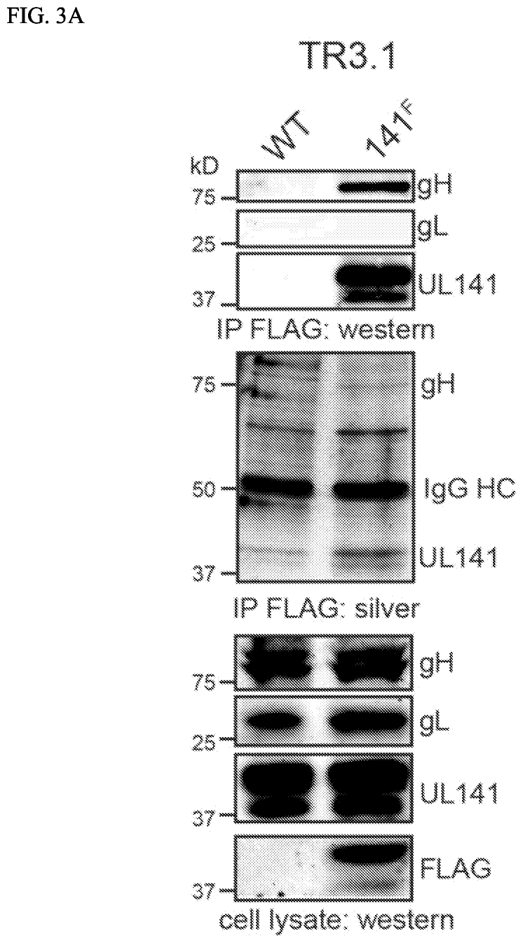

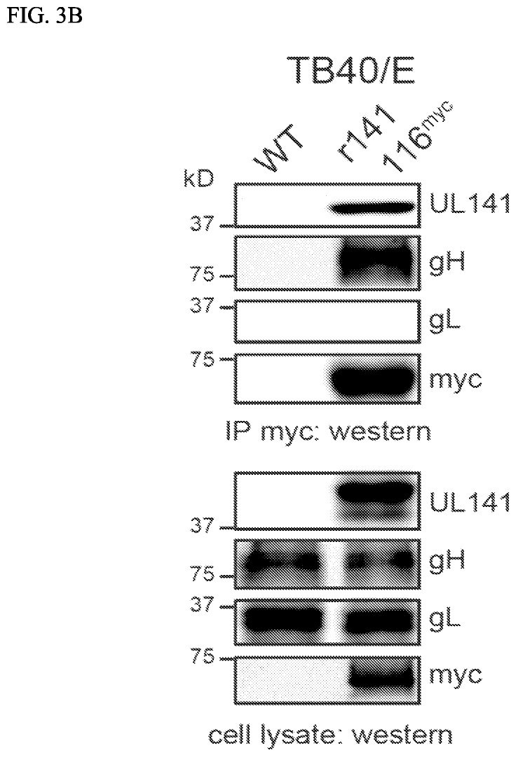

Ectopic expression of HCMV gB together with gH/gL is sufficient to drive cell-cell fusion, leading to the formation of syncytia^{56,57}, as was first shown with expression of herpes simplex virus-1 gH, gL, gD, and gB⁵⁸. gH/UL116 is the only ‘gL-less’ gH complex currently reported¹⁹ in HCMV, and results show that UL141 can also join this complex (FIG. 3B). However, no data currently exists showing that gH-dependent, gB-catalyzed membrane fusion can occur in the absence of gL. Because UL141/gH/UL116 can enhance pentamer-independent infectivity of ARPE-19 cells, this complex may function together with gB to fuse membranes, and this fusion requires the contribution of a UL141 binding partner.

Adenovirus Vector Based Cell Fusion Assays

Although syncytia (multi-nucleated cell bodies which form after cell fusion) can normally be visualized and counted by standard light microscopy, this approach is relatively nonquantitative. This is because the size and number of nuclei per individual syncytia often varies based upon the relative fusogenic capacity of specific viral envelope protein complexes and their cellular receptors. Consequently, a fluorescence-based assay is used to robustly quantify cell-cell fusion that is similar to one published for studying HCMV gB variants⁵⁹, Initially, RPE-1 cells stably expressing either GFP or mCherry are mixed together at a 1:1 ratio and then co-transduced with adenovirus (Ad) vectors expressing gH, UL116, UL141, and gB, analogous to the approach used successfully for syncytia studies with HCMV gH/gL+gB56,57. RPE-1 is used because they can support UL141-dependent entry. An Ad vector system from Microbix, Inc. is used, in which LacI-mediated repression of the transgenes is used during amplification of the vector to avoid potential cytotoxic effects. Ad vectors expressing gH/gL+gB, or vesiculostomatitis virus G protein (VSV G), serve as positive controls. Incomplete combinations lacking gH or gB serve as negative controls, alongside empty Ad vectors. Syncytia formation in fluorescent micrographs is quantified by Imaris imaging software after fluorescence detection of GFP+/mCherry+ double-positive foci ive cell monolayers. This approach is also applied to test for syncytia formation in additional cell types, including engineered ARPE-19, found to be permissive for UL141-dependent entry, as described above. Abolishment of fusion by siRNA knockdown of identified UL141 receptors is also tested.

The UL141/gH/UL116 complex may induce syncytia when co-expressed with gB in RPE-1, given the observed UL141-dependent HCMV entry into this cell type. Additionally, some UL141 binding partners may show differing abilities to catalyze syncytia formation due to their ability to cluster/diffuse within cellular membranes. In this regard, if TRAIL-R2 catalyzes cell-cell fusion TRAIL-R2:GPI is also tested, given it lacks a transmembrane domain. When cell fusion is observed, purified soluble UL141/gH/UL116 or UL141-Fc protein is tested for blocking fusion, which may be unlikely.

UL141-dependent HCMV entry into ARPE-19 might be a two-step process where virions are first endocytosed via UL141 receptors, but fusion and cytoplasmic release then occurs via a canonical gB+gH/gL mediated process. The above-described study determines whether this gH complex+gB suffices for fusion. The recently identified naturally occurring hyper-fusogenic variants of gB⁵⁹ may be used to address whether UL141/gH/UL116 can potentially function as a weak fusion initiator. If needed, flow cytometry is an alternative to fluorescent micrographs to quantify syncytia⁶¹.

### Example 4: Discussion

The studies described herein illuminate roles for the newly identified viral envelope glycoprotein complex that may play a direct role in HCMV entry. Other studies include (i) solving the 3D structure of purified UL141/gH/UL116 to visualize the native complex in virions by cryoEM, and (ii) addressing the utility of UL141/gH/UL116 as a vaccine antigen and/or a target for neutralizing-antibody drug development.

Because the pentamer can bind unique receptors and mediate viral entry into several cell types that the trimer cannot, the encoding of a third gH complex by HCMV is investigated. UL141 may support entry when antiviral defenses operate to neutralize/dampen other routes of HCMV entry into cells. For instance, interferon (IFN)⁶² is known to induce expression of both TRAIL and TRAIL-DRs⁶³⁻⁶⁶. In turn, TRAIL-R2 signaling activates a unique mode of clathrin-mediated endocytosis (CME) driven by Dynamin-1^{67,68}. Given the high-affinity interaction of UL141 with TRAIL-R2^{21,28,29}, perhaps the UL141/gH/UL116 complex can utilize TRAIL-R2 as an entry receptor when IFN, TRAIL or other innate-defense pathways are activated in vivo. In summary, the UL141/gH/UL116 may function as a ‘fine tuner’ of viral entry into select cell types and/or under specific immunological conditions.

### Example 5: The Human Cytomegalovirus Immune Evasion Protein UL141 is Incorporated into Virions in a Ternary Complex with Glycoprotein H and UL116

In herpesviruses, the core viral machinery for cell entry includes glycoproteins H (gH) and L (gL), which assemble into a heterodimer that regulates membrane fusion during entry. Human cytomegalovirus (HCMV), however, expresses at least three gH complexes, two in which gL links via a disulfide bond to either of two alternative receptor binding moieties, glycoprotein O or UL128/UL130/UL131. In 2016, a third complex was discovered, in which gH associates with UL116 instead of gL. Despite its presence in virions, no function has yet been identified for the gH/UL116 complex. Here, it is shown that the viral immune evasion protein UL141 is incorporated into virions in a ternary complex with gH and UL116. Endoglycosidase H-resistant UL141 species are readily detected in virions, and co-immunoprecipitation results from infected cells indicate a complex between UL141, gH, and UL116. Expression of the ectodomains of UL141 and gH together with UL116 results in secretion of a three-part complex that was purified, fractionated by fast performance liquid chromatography and imaged to ˜ 20 Å resolution by negative staining electron microscopy. Replacement of the defective UL128-UL131 locus in the HCMV strain AD169 with UL141 resulted in a virus that showed a cell-to-cell spread advantage on ARPE-19 epithelial cells relative to the parental virus, which lost UL141 during tissue culture adaptation. These findings demonstrate that the gH/UL116 complex contains a third component, UL141, and are consistent with a role in cell-to-cell spread. Given that UL141 exhibits high affinity binding to TRAIL death receptors and CD155, without being bound to a particular theory, it is suggested that UL141 is the receptor binding moiety for the gH/UL116 complex.

Significance

How viruses develop the receptor binding interactions that propagate their entry into target cells remains largely a matter of speculation. UL141 is a cytomegalovirus glycoprotein that facilitates viral evasion of cell mediated immunity by intercepting immune signaling molecules to prevent them from reaching the cell surface. For example, UL141 binds to TNF-related apoptosis inducing ligand (TRAIL) death receptors to thwart their journey to the cell surface. Here, it is shown that UL141 assembles as the third component for the newly identified HCMV envelope glycoprotein complex, gH/UL116. Selection pressures evidently have favored the development of viral ER-resident glycoproteins that tightly bind immune signaling receptors to impede their journey to the cell surface. Without wishing to be bound by scientific theory, these results suggest an example in which a binding interface involved in immune evasion can play a dual role as receptor binding moiety during cell entry.

Introduction

As in other enveloped viruses, HCMV enters cells via fusion of viral and target cell membranes. The core membrane fusion machinery shared across the herpesviruses is comprised of a heterodimer of glycoprotein H (gH)/glycoprotein L (gL), together with glycoprotein B (gB)(1), where gH/gL is posited to engage host cell surface receptors to regulate gB catalyzed membrane fusion. HCMV gH is a component of two different gH/gL complexes found on the virion envelope. HCMV gL is derivatized in the ER at Cys144, dictating its assembly into gH/gL/gO (trimer) or gH/gL/UL128/UL130/UL131 (pentamer) (2-4). These distinct gH/gL complexes can utilize different cell surface receptors, with PDGFRα being a receptor for the trimer (5-7) and the pentamer utilizing neuropilin-2 (NRP2) (8) and OR14I1 (9) to enter cells in a CD147-dependent fashion (10). The trimer is required for the infectivity of cell-free virions (11, 12), consistent with its expression being stably maintained in HCMV strains repeatedly passaged in fibroblasts. In contrast, virtually all fibroblast-adapted ‘laboratory strains’ acquire mutations in UL128, UL130, or UL131, abolishing viral tropism for non-fibroblasts; repairing these mutations restores infectivity for epithelial cells, endothelial cells, and leukocytes (13-15).

In 2016, a third virion-incorporated, gH-containing complex was reported by GSK Vaccines (16). They showed that a gH/UL116 heterodimer is formed, but unlike gL, UL116 is not disulfide linked to gH. Despite its abundance in virions (16, 17), a biological role for the gH/UL116 complex has remained elusive. Here, the inventors show that the viral immunoevasin UL141 assembles as a third member of the gH/UL116 complex, and is incorporated into HCMV virions.

Results

HCMV clinical strains incorporate UL141 into virions.

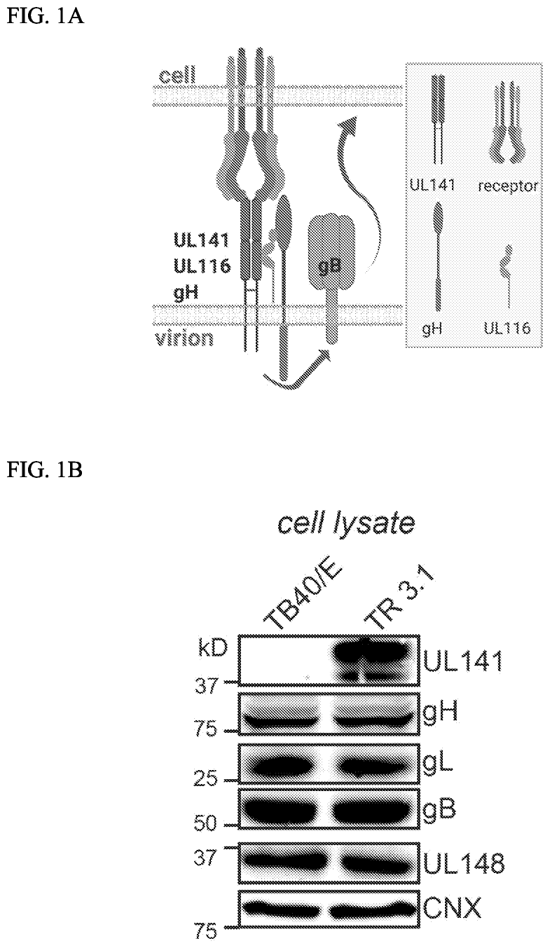

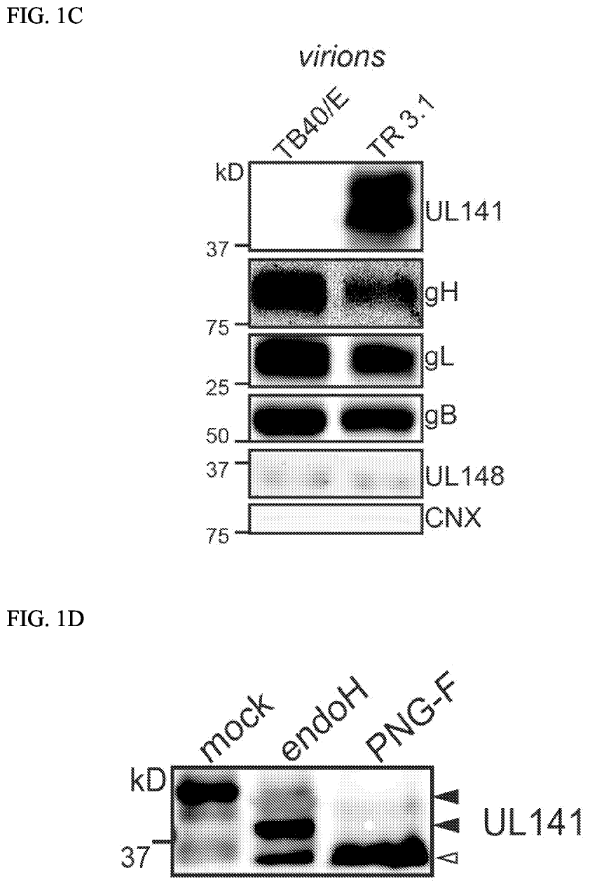

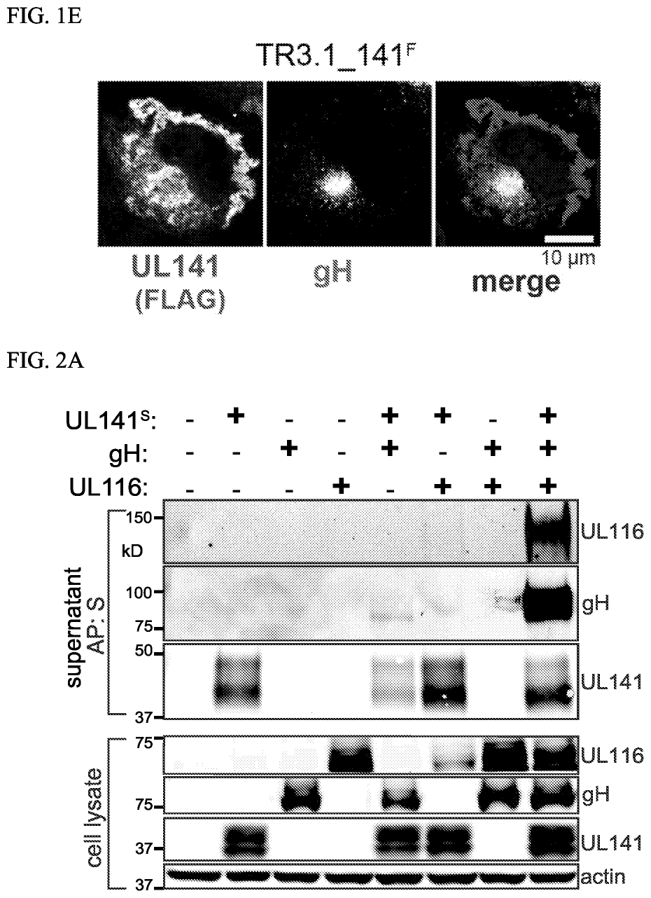

An infectious bacterial artificial chromosome (BAC) clone of HCMV strain TR3.1, which is intact for all known viral genes encoded by primary HCMV isolates (18), was compared to the BAC-cloned TB40/E strain for the relative incorporation of gH, gL and gB into the virion envelope. All three of these canonical envelope proteins were present in virions of these two strains at roughly similar levels (FIG. 1C). To assess the purity of these virion preparations, the two ER-localized HCMV immunoevasins UL141 and UL148 were also analyzed, as well as the cellular ER-marker calnexin (CXN). As expected (19-21), UL148 was abundant in infected cell lysates but largely excluded from virions of both strains (FIGS. 1B-1C). Surprisingly, however, robust levels of UL141 were incorporated into TR3.1 virions. No UL141 expression was detected in TB40/E virions or infected cells, as expected since TB40/E contains a frameshift mutation in UL141 (22, 23). Since UL141 can reside within the ER, where it sequesters TRAIL-DRs and CD155 (23, 24), the inventors treated TR3.1 virions with endoglycosidase H (endoH) and protein N-glycosidase F (PNGaseF) to assess the maturation status of its N-glycans. Indeed, a substantial portion of the UL141 detected in TR3.1 virions was endoH-resistant (FIG. 1D), indicating it had trafficked through the Golgi, as would be expected for a virion-incorporated envelope protein. In addition, confocal microscopy revealed a significant portion of UL141 in close association with the cytoplasmic virion assembly compartment (VAC) (FIG. 1E), a virally-induced intracellular membrane structure where envelope glycoproteins such as gH are incorporated into maturing viral particles (16, 25).

Assembly of a UL141/gH/UL116 Complex in Transfected and Infected Cells.

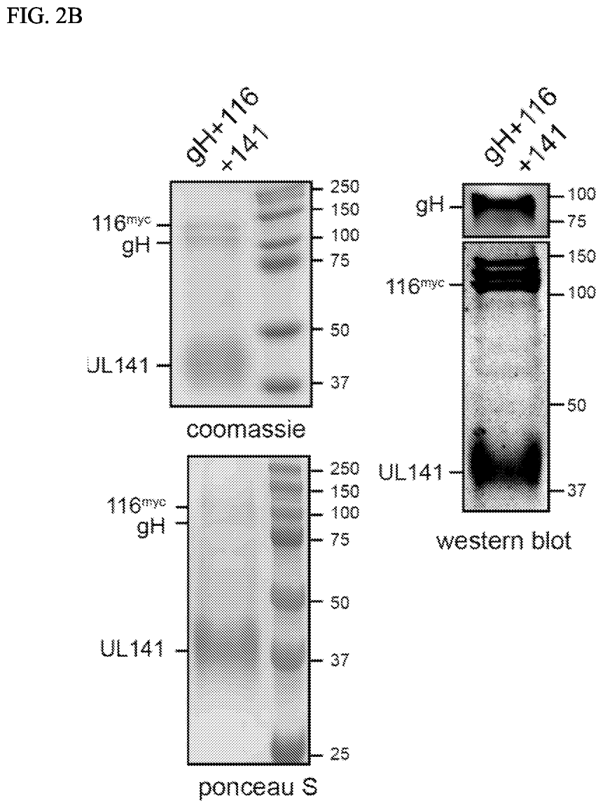

A recent viral proteome-wide mass spectrometry study reported an unbiased inventory of virus-virus and virus-host protein-protein interactions that occur within infected cells (26). Results from the study indicate that gH is found in UL116 immunoprecipitates (IPs), in agreement with the 2016 study that originally reported the complex (16). Intriguingly, both gH and UL116 were also detected in UL141 IPs. Although many other proteins were also detected in UL116 IPs (e.g. PDGFRα and UL130), the inventors hypothesized that UL141 might be a previously unidentified third component of the gH/UL116 complex. To test this, the inventors transfected HEK-293T cells with plasmids encoding UL116, the gH ectodomain, and a polyhistidine-tagged UL141 ectodomain, and subjected supernatants to immobilized-protein affinity purification directed against an S-tag fused to the C-terminus of the UL141 ectodomain. These analyses revealed high level secretion of a UL141/gH/UL116 complex from transfected cells (FIG. 2A). A octahistidine-tagged UL141 ectodomain expression construct was subsequently developed and carried out larger scale expression in Expi293F cells. Using this approach the inventors were able to obtain recombinant complex on the scale of hundreds of micrograms per liter, and further confirmed the identity of the purified species by Western blot (FIG. 2B).

To test whether a gH/UL116/UL141 complex forms in infected cells, aTR3.1 virus encoding UL141 fused to a C-terminal FLAG tag was constructed and compared this to wild-type TR3.1. Anti-FLAG immunoprecipitation pulled down a protein species of the expected size reactive with an anti-UL141 monoclonal antibody (mAb) (24, 27) and gH co-immunoprecipitated with UL141 while gL did not (FIG. 3A). Reciprocally, analyses of cells infected with strain TB40/E repaired for UL141 expression and encoding a myc-tagged UL116 revealed co-immunoprecipitation of gH and UL141, but not gL (FIG. 3B). Collectively, these results suggest the existence of a UL141/gH/UL116 complex that is incorporated into HCMV virions.

A strain AD169 derivative restored for UL141 shows evidence of enhanced cell-to-cell spread.

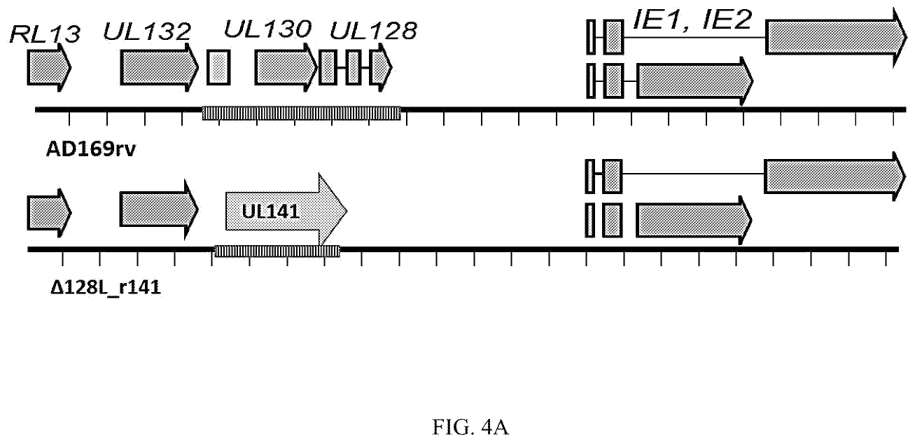

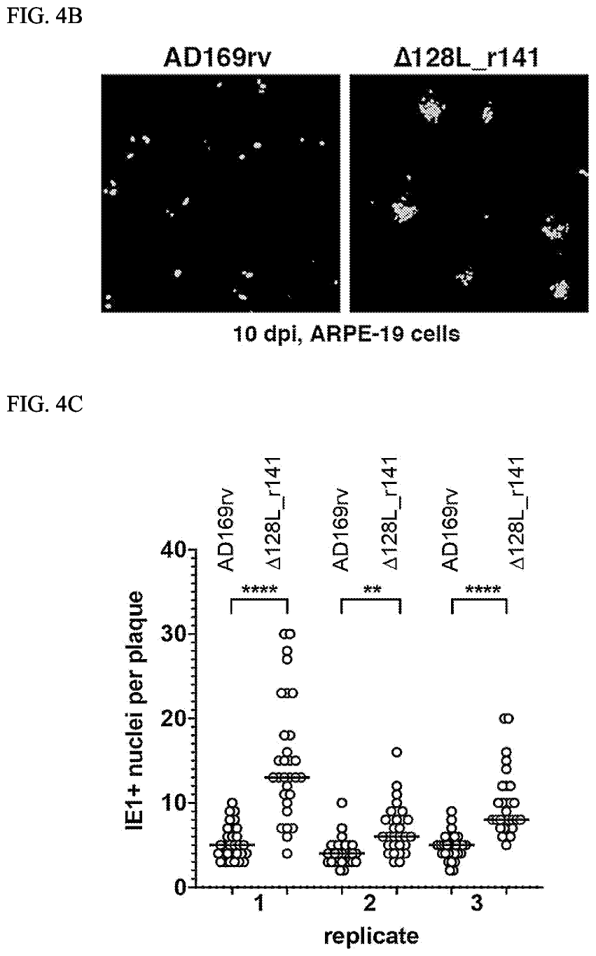

To address whether virion-incorporated UL141 might play a role in cell entry, a derivative of HCMV strain AD169rv, which ordinarily lacks UL141, was engineered in which the UL128 locus, UL128-UL131, was replaced with the UL141 coding sequence from strain TR3.1 (FIG. 4A). The inventors chose to replace the UL128 locus with UL141 because AD169rv already lacks the capacity to express a functional pentamer (gH/gL/UL128-131) from this locus, owing to a frameshift mutation in UL131 (4, 28). Therefore, replacing the entire UL128 locus with the UL141 coding sequence here would provide a promoter and polyadenylation signal and if anything, would be expected to produce a gain-of-function phenotype since AD169 is already devoid of pentamer.

The UL141-restored virus, dubbed Δ128L_r141, was compared to parental AD169rv for its ability to infect and form plaques on ARPE-19 epithelial cells. AD169rv poorly infects epithelial cells due to its inability to express the pentamer. Accordingly, it was observed that even after 10 days postinfection (dpi) at an MOI of 0.1 TCID₅₀/cell, parental AD169rv virus showed only small foci of infected cells that averaged only 4 or 5 infected cells per focus (FIG. 4B). At this same time point, in each of three independent biological replicates the UL141-expressing AD169rv virus, Δ128L_r141, showed a statistically significant increase in the number of cells per focus (FIG. 4B). However, there were no obvious differences in the number of foci (FIG. 4C). From these results, the inventors concluded that the two viruses appear to enter ARPE-19 cells with similarly low efficiency, but the virus restored for UL141 shows an advantage in cell-to-cell spread.

Taken together, these findings suggest that the viral glycoprotein UL141, which was previously thought to be confined to the endoplasmic reticulum (23), plays a dual role as a component of a virion glycoprotein H complex.

Discussion

It is surprising to find that UL141 is incorporated into the virion envelope. UL141 was originally identified to function within the ER as a viral immune evasion factor, and it has been well established that viral envelope glycoproteins derive from post-trans Golgi network (TGN) compartments. Nonetheless, given that UL141 binds with high affinity to TNF-related apoptosis inducing ligand (TRAIL) death receptors (DR) and to CD155 (PVR), its presence as a component of a gH complex may suggest novel roles in cell entry as a receptor binding moiety, or in cell to cell spread of HCMV. Indeed, even though the gH/UL116 complex was first reported in 2016, a raison d'être for the novel gH complex has remained elusive. The incorporation of UL141 as the third component of the gH/UL116 complex would provide a plausible receptor binding moiety with a role in entry or cell to cell spread.

Without wishing to be bound by scientific theory, these results suggest that the mechanism underlying enhanced cell-to-cell spread involves binding of the UL141/gH/UL116 complex to one or more of the same cellular gene products on the surface of a target cells that UL141 binds and retains in the context of its role as an ER-based immunoevasin. In this way, treating or vaccinating against CMV or HCMV may, without being bound to a particular theory, function in that an immunogenic composition including a the HCMV protein UL141 or a fragment thereof, or a nucleic acid encoding the same is given for the prevention or treatment of HCMV.

UL141 interferes with surface presentation of CD112 (Nectin-2), CD155 (PVR), and the TRAIL death receptors (TRAIL DR). However, the TRAIL-DR are favored as receptors for the UL141/gH/UL116 complex during entry for three main reasons. Firstly, UL141 is not sufficient to downregulate CD112. TRAIL-R1 and TRAIL-R2, are upregulated by interferons (29-32). Hence, the presence of UL141 in the virion envelope in a gH complex may suggest that HCMV utilizes interferon stimulated gene (ISG) products, such as the TRAIL-DR as an entry receptor for cell-to-cell spread. Because viruses are notorious for triggering interferon secretion, the milieu surrounding infected cells is expected to be upregulated for ISGs such as TRAIL-DR. Since most ISG are antiviral, it makes sense for a virus to ‘turn the table’ on the host to exploit an ISG, such as the TRAIL-DR, to facilitate spread in the context of the interferon response.

Materials and Methods

Cells

Human telomerase immortalized human foreskin fibroblasts, prepared from primary HFF cells (ATCC, Cat #SCRC-1041) as previously described&. ARPE-19 retinal pigment epithelial cells were purchased from ATCC (Cat #CRL-2302). HEK-293T cells were purchased from Genhunter Corp. (Nashville, TN). All cells were cultured in Dulbecco's Modified Eagle's Medium (DMEM, Corning) supplemented with 25 μg/mL gentamicin (Invitrogen), 10 μg/mL ciprofloxacin (Genhunter), and either 5-10% fetal bovine serum (FBS, Sigma #F2442) or 5% newborn calf serum (NCS, Sigma #N4637 or Gemini Bio GemCell).

Viruses

Viruses were reconstituted by electroporation of HCMV bacterial artificial chromosomes (BACs) into HFFT. Parental TB40/E, TR3.1, and derivatives were amplified at low MOI on HFFT until extensive CPE was observed. AD169rv and related recombinants virus was amplified at low MOI on ARPE-19 cells until 95-100% cytopathic effect (CPE) was observed. Virus-containing culture supernatants were then subjected to centrifugation (1000 g) for 10 minutes to pellet any cellular debris. Cell-associated virus was then released by Dounce-homogenization of pelleted infected cells, clarified of cell debris by centrifugation (1000 g, 10 min), and combined with the cell-free medium then ultracentrifuged (85,000 g, 1 h, 4° C.) through a 20% sorbitol cushion, and the resulting virus pellet was resuspended in DMEM containing 20% NCS or FBS.

Virus Titration

Infectivity of virus stocks and samples were determined by the tissue culture infectious dose 50% (TCID₅₀) assay. Briefly, serial dilutions of virus were used to infect multiple wells of a 96-well plate. After 12 days, wells were scored as positive or negative for CPE, and TCID₅₀ values were calculated according to the Spearman-Kirber method.

Plasmids and Recombinant HCMVs

Unless otherwise noted, all restriction enzymes and cloning reagents were obtained from New England Biolabs (NEB, Ispwitch, MA), all DNA polymerase for PCR was KOD Hot Start (MilliporeSigma Cat #710863) supplemented with 1.3 M betaine monohydrate, and all oligonucleotides and synthetic DNA fragment (gBlocks) were obtained from Integrated DNA Technologies (Coralville, IA). Additionally, all synthetic gBlocks were codon optimized according to Homo sapiens codon bias, using gBlock codon optimization settings at IDTDNA.com. A UL141 ectodomain expression construct was prepared by linearizing the adenovirus-5 shuttle plasmid pDC515(io) (Microbix Biosystems, Inc) using EcoRI. NEB HiFI Assembly mix to insert a synthetic dsDNA fragment (IDT gBlock) termed ‘spUL141ecto_S_Gibs,’ such that the encoded UL141 ectodomain coding sequence was positioned downstream of an MCMV promoter and upstream of an SV40 polyadenylation signal. To provide more detail, the gBlock encodes at its 5′-end the first 18 amino acids of human serum albumin (MKWVTFISLLFLFSSAYS; NCBI Reference Sequence: NP_000468.1), which serve as a heterologous signal peptide, followed by amino acids 30-279 of HCMV UL141 (UniProt.org entry Q6RJQ3, or NCBI Reference Sequence: YP_081575.1), followed by the amino acid sequence GSGGGS (a linker), an S-tag, KETAAAKFERQHMDS (to facilitate purification and detection) and a termination codon (TAG).

To construct a plasmid encoding the ectodomain of HCMV glycoprotein H (gH), a pEF1/V5 expression plasmid (Invitrogen) was linearized with EcoRV and a synthetic gBlock encoding amino acids 1-709 of HCMV gH (GenBank: ABV71597.1) from strain TB40 fused at the carboxy terminus to the amino acids GGSGGSDYKDDDDK, which provide a Gly-Ser rich linker and a FLAG epitope tag (DYKDDDDK). The gH ectodomain gBlock coding sequence then terminates using TGA stop codon.

Similarly, a synthetic gBlock encoding UL116 fused to a Gly-Ser linker and terminating in a C-terminal myc epitope tag was similarly assembled using NEB HiFi Assembly into EcoRV linearized pEF1/V5 plasmid. This gBlock encodes the entire 313 a.a. UL116 protein (GenBank: ABV71630.1) from HCMV strain TB40 fused at its C-terminus to the amino acid sequence GGSGGSEQKLISEEDL, which provides a six amino acid Gly-Ser rich linker and a Myc eptiope tag (EQKLISEEDL). This gBlock terminates in a TGA stop codon.

For all new plasmids, “NEB5alpha” competent cells (New England Biolabs) were transformed with the assembly reaction and ampicillin resistant colonies were isolated on LB agar plates; plasmid DNA was isolated from single colonies carrying plasmid clones and were verified by Sanger sequencing (Genewiz, Inc.) to contain desired insert sequence free of spurious mutations.

The TR3 BAC (also known as TR3.1), a BAC clone of the clinical HCMV strain TR that has been fully restored to wild-type status and to which ganciclovir sensitivity was restored, was a generous gift of Dr. Klaus Frueh, OHSU, Beaverton, Oregon⁴¹. Its full sequence is found as GenBank Accession number MN075802.1. The inventors constructed a GFP-tagged TR3.1 by BAC mutagenesis. The inventors first used en passant BAC recombineering to insert an excisable kanamycin resistance allele into the GFP expression cassette located between US34 and TRS1 in a BAC-cloned GFP expressing TB40E derivative that was previously constructed by Umashankar et al.³³, which is now commonly called “TB40E_5. This procedure entailed using primers EGFP_in_Kan_Fw and EGFP_in_Kan_Rv to PCR amplify a kanamycin resistance cassette that contains an ISceI recognition site at its 5′ end (I-SceI-Kan). The inventors then electroporated the DpnI digested PCR product into Escherichia coli GS1783 carrying the TB40E_5 BAC. After confirming that desired recombination event had occurred, a new PCR product containing the GFP cassette and portions of US34 and the US34/TRS1 intergenic region was prepared by PCR, using primers Us34CT_Fw and TRS1/Us34 Reg Rv. This PCR product was electroporated into E. coli GS1783 containing the TR3 BAC. Kanamycin resistant colonies were picked, grown up, confirmed and then resolved to remove the I-SceI-Kan cassette. The final BAC was confirmed by sequencing at MiGS Center (Pittsburgh, PA).

Similar techniques were used to insert a FLAG epitope tag at the C-terminal cytoplasmic tail of UL141.

### Example 6: Blockade of the 3-Mer (UL141/UL116/gH) Envelope Glycoprotein Complex Results in Reduced Entry and/or Spread of HCMV

For investigating the ability of anti-3-mer (e.g. anti-UL141/UL116/gH) antisera to block initial entry of HCMV into cells, anti-3-mer sera, anti-2-mer polyclonal sera or preimmune generated in rabbits is preincubated with HCMV particles encoding all genetic components of the 3-mer (UL141, gH and UL116), those lacking UL141 or those lacking UL116, for 2 hours (h) at room temperature. Virus is then added to epithelial cells (ARPE-19), normal human fibroblasts (NHDF), a human myeloid cell line (THP-1) that was differentiated or not with PMA, endothelial cells (HUVEC or tHUVEC), dendritic cells (IL-4/GMCSF derived or Flt-3 derived) from human peripheral blood, human liver sinusoidal endothelial cells (LSEC) and other relevant human cells lines. After the 2 h pre-incubation, virus is added to cell cultures including similar levels of antibody. Anti-gH monoclonal antibody (clone MSL-109)³⁴ is added as a positive control for neutralization.

To test the ability of anti-3-mer antisera to block cell-to-cell spread, experiments described above are repeated without preincubation of viral particles with antisera. Instead, anti-sera are added at various concentration to cell cultures infected 48 h earlier at various concentrations in order to assess whether they could block viral spread, independent of neutralizing initial HCMV entry.

Monoclonal antibodies selected for specific reactivity with the 3-mer, 2-mer, or both are added to virus and/or cell cultures as described above to assess their relative abilities to neutralize initial HCMV entry and/or to inhibit cell-to-cell spread, as described.

### REFERENCES FOR EXAMPLES 1-4

1. Boeckh, M. & Geballe, A. P. Cytomegalovirus: pathogen, paradigm, and puzzle. J. Clin. Invest. 121, 1673-1680, doi:10.1172/JCI45449 (2011).
2. Cannon, M. J. Congenital cytomegalovirus (CMV) epidemiology and awareness. J. Clin. Virol. 46 Suppl 4, S6-10, doi:10.1016/j.jcv.2009.09.002 (2009).
3. Modlin, J. F. et al. Vaccine development to prevent cytomegalovirus disease: report from the National Vaccine Advisory Committee. Clin. Infect. Dis. 39, 233-239 (2004).
4. Heldwein, E. E. gH/gL supercomplexes at early stages of herpesvirus entry. Curr. Opin. Virol. 18, 1-8, doi:10.1016/j.coviro.2016.01.010 (2016).
5. Chandramouli, S. et al. Structural basis for potent antibody-mediated neutralization of human cytomegalovirus. Sci Immunol 2, doi:10.1126/sciimmunol.aan1457 (2017).
6. Ciferri, C. et al. Structural and biochemical studies of HCMV gH/gL/gO and Pentamer reveal mutually exclusive cell entry complexes. Proc. Natl. Acad. Sci. U.S.A., doi:10.1073/pnas.1424818112 (2015).
7. Wang, D. & Shenk, T. Human cytomegalovirus virion protein complex required for epithelial and endothelial cell tropism. Proc. Natl. Acad. Sci. U.S.A. 102, 18153-18158, doi:10.1073/pnas.0509201102 (2005).
8. Kabanova, A. et al. Platelet-derived growth factor-alpha receptor is the cellular receptor for human cytomegalovirus gHgLgO trimer. Nat Microbiol 1, 16082, doi:10.1038/nmicrobiol.2016.82 (2016).
9. Wu, K., Oberstein, A., Wang, W. & Shenk, T. Role of PDGF receptor-alpha during human cytomegalovirus entry into fibroblasts. Proc. Natl. Acad. Sci. U.S.A., doi:10.1073/pnas.1806305115 (2018).
10. Wu, Y. et al. Human cytomegalovirus glycoprotein complex gH/gL/gO uses PDGFR-alpha as a key for entry. PLoS Pathog. 13, e1006281, doi:10.1371/journal.ppat.1006281 (2017).
11. Martinez-Martin, N. et al. An Unbiased Screen for Human Cytomegalovirus Identifies Neuropilin-2 as a Central Viral Receptor. Cell 174, 1158-1171 e1119, doi:10.1016/j.cell.2018.06.028 (2018).
12. E, X. et al. OR14I1 is a receptor for the human cytomegalovirus pentameric complex and defines viral epithelial cell tropism. Proc. Natl. Acad. Sci. U.S.A. 116, 7043-7052, doi:10.1073/pnas.1814850116 (2019).
13. Vanarsdall, A. L. et al. CD147 Promotes Entry of Pentamer-Expressing Human Cytomegalovirus into Epithelial and Endothelial Cells. MBio 9, e00781-00718, doi:10.1128/mBio.00781-18 (2018).
14. Jiang, X. J. et al. UL74 of human cytomegalovirus contributes to virus release by promoting secondary envelopment of virions. J. Virol. 82, 2802-2812, doi:10.1128/JVI.01550-07 (2008).
15. Wille, P. T., Knoche, A. J., Nelson, J. A., Jarvis, M. A. & Johnson, D. C. A human cytomegalovirus gO-null mutant fails to incorporate gH/gL into the virion envelope and is unable to enter fibroblasts and epithelial and endothelial cells. J. Virol. 84, 2585-2596, doi:10.1128/JVI.02249-09 (2010).
16. Zhou, M., Lanchy, J. M. & Ryckman, B. J. Human Cytomegalovirus gH/gL/gO Promotes the Fusion Step of Entry into All Cell Types, whereas gH/gL/UL128-131 Broadens Virus Tropism through a Distinct Mechanism. J. Virol. 89, 8999-9009, doi:10.1128/JVI.01325-15 (2015).
17. Ryckman, B. J., Jarvis, M. A., Drummond, D. D., Nelson, J. A. & Johnson, D. C. Human cytomegalovirus entry into epithelial and endothelial cells depends on genes UL128 to UL150 and occurs by endocytosis and low-pH fusion. J. Virol. 80, 710-722, doi:10.1128/JVI.80.2.710-722.2006 (2006).
18. Hahn, G. et al. Human cytomegalovirus UL131-128 genes are indispensable for virus growth in endothelial cells and virus transfer to leukocytes. J Virol 78, 10023-10033, doi:10.1128/JVI.78.18.10023-10033.2004 (2004).
19. Calo, S. et al. The Human Cytomegalovirus UL116 Gene Encodes an Envelope Glycoprotein Forming a Complex with gH Independently from gL. J. Virol. 90, 4926-4938, doi:10.1128/JVI.02517-15 (2016).
20. Siddiquey, M. N. A. et al. The HCMV Glycoprotein L Analog UL116 Interacts With Viral ER Resident Glycoprotein UL148 and Promotes Virion Incorporation of gH/gL Complexes. 44th Annual International Herpesvirus Workshop, Knoxville, TN, Oral Abstract 5.28 (2019).
21. Smith, W. et al. Human cytomegalovirus glycoprotein UL141 targets the TRAIL death receptors to thwart host innate antiviral defenses. Cell Host Microbe 13, 324-335, doi:10.1016/j.chom.2013.02.003 (2013).
22. Tomasec, P. et al. Downregulation of natural killer cell-activating ligand CD155 by human cytomegalovirus UL141. Nat. Immunol. 6, 181-188, doi:10.1038/ni1156 (2005).
23. Prod'homme, V. et al. Human cytomegalovirus UL141 promotes efficient downregulation of the natural killer cell activating ligand CD112. J. Gen. Virol. 91, 2034-2039, doi:10.1099/vir.0.021931-0 (2010).
24. Picarda, G. et al. Cytomegalovirus Evades TRAIL-Mediated Innate Lymphoid Cell 1 Defenses. J. Virol. 93, doi:10.1128/JVI.00617-19 (2019).
25. Verma, S. et al. Inhibition of the TRAIL death receptor by CMV reveals its importance in NK cell-mediated antiviral defense. PLoS Pathog. 10, e1004268, doi:10.1371/journal.ppat.1004268 (2014).
26. Borza, C. M. & Hutt-Fletcher, L. M. Alternate replication in B cells and epithelial cells switches tropism of Epstein-Barr virus. Nat Med 8, 594-599, doi:10.1038/nm0602-594 (2002).
27. Hahn, A. S. et al. A Recombinant Rhesus Monkey Rhadinovirus Deleted of Glycoprotein L Establishes Persistent Infection of Rhesus Macaques and Elicits Conventional T Cell Responses. J. Virol. 94, doi:10.1128/JVI.01093-19 (2020).
28. Nemcovicova, I., Benedict, C. A. & Zajonc, D. M. Structure of human cytomegalovirus UL141 binding to TRAIL-R2 reveals novel, non-canonical death receptor interactions. PLoS Pathog. 9, e1003224, doi:10.1371/journal.ppat.1003224 (2013).
29. Nemcovicova, I. & Zajonc, D. M. The structure of cytomegalovirus immune modulator UL141 highlights structural Ig-fold versatility for receptor binding. Acta Crystallogr. D Biol. Crystallogr. 70, 851-862, doi:10.1107/S1399004713033750 (2014).
30. Stampfer, S. D. & Heldwein, E. E. Stuck in the middle: structural insights into the role of the gH/gL heterodimer in herpesvirus entry. Curr. Opin. Virol. 3, 13-19, doi:10.1016/j.coviro.2012.10.005 (2013).
31. Caposio, P. et al. Characterization of a live-attenuated HCMV-based vaccine platform. Sci. Rep. 9, 19236, doi:10.1038/s41598-019-55508-w (2019).
32. Li, G., Nguyen, C. C., Ryckman, B. J., Britt, W. J. & Kamil, J. P. A viral regulator of glycoprotein complexes contributes to human cytomegalovirus cell tropism. Proc. Natl. Acad. Sci. U.S.A. 112, 4471-4476, doi:10.1073/pnas.1419875112 (2015).
33. Siddiquey, M. N. A., Zhang, H., Nguyen, C. C., Domma, A. J. & Kamil, J. P. The Human Cytomegalovirus Endoplasmic Reticulum-Resident Glycoprotein UL148 Activates the Unfolded Protein Response. J Virol 92, doi:10.1128/JVI.00896-18 (2018).
34. Zhang, H. et al. The Human Cytomegalovirus Nonstructural Glycoprotein UL148 Reorganizes the Endoplasmic Reticulum. mBio 10, doi:10.1128/mBio.02110-19 (2019).
35. Sinzger, C. et al. Cloning and sequencing of a highly productive, endotheliotropic virus strain derived from human cytomegalovirus TB40/E. J Gen Virol 89, 359-368, doi:10.1099/vir.0.83286-0 (2008).
36. Sanchez, V., Greis, K. D., Sztul, E. & Britt, W. J. Accumulation of virion tegument and envelope proteins in a stable cytoplasmic compartment during human cytomegalovirus replication: characterization of a potential site of virus assembly. J. Virol. 74, 975-986 (2000).
37. Nobre, L. V. et al. Human cytomegalovirus interactome analysis identifies degradation hubs, domain associations and viral protein functions. Elife 8, doi:10.7554/eLife.49894 (2019).
38. Wang, D. & Shenk, T. Human cytomegalovirus UL131 open reading frame is required for epithelial cell tropism. J. Virol. 79, 10330-10338, doi:10.1128/JVI.79.16.10330-10338.2005 (2005).
39. Stegmann, C. et al. A derivative of platelet-derived growth factor receptor alpha binds to the trimer of human cytomegalovirus and inhibits entry into fibroblasts and endothelial cells. PLoS Pathog. 13, e1006273, doi:10.1371/journal.ppat.1006273 (2017).
40. Nguyen, C. C., Siddiquey, M. N. A., Zhang, H., Li, G. & Kamil, J. P. Human Cytomegalovirus Tropism Modulator UL148 Interacts with SEL1L, a Cellular Factor That Governs Endoplasmic Reticulum-Associated Degradation of the Viral Envelope Glycoprotein gO. J. Virol. 92, e00688-00618, doi:10.1128/JVI.00688-18 (2018).
41. Siddiquey, M. N. A., Zhang, H., Nguyen, C. C., Domma, A. J. & Kamil, J. P. The Human Cytomegalovirus Endoplasmic Reticulum-Resident Glycoprotein UL148 Activates the Unfolded Protein Response. J. Virol. 92, e00896-00818, doi:10.1128/JVI.00896-18 (2018).
42. Ostermann, E., Spohn, M., Indenbirken, D. & Brune, W. Complete Genome Sequence of a Human Cytomegalovirus Strain AD169 Bacterial Artificial Chromosome Clone. Genome Announc 4, doi:10.1128/genomeA.00091-16 (2016).
43. Sheikh, M. S. et al. p53-dependent and -independent regulation of the death receptor KILLER/DR5 gene expression in response to genotoxic stress and tumor necrosis factor alpha. Cancer Res. 58, 1593-1598 (1998).
44. Yamaguchi, H. & Wang, H. G. CHOP is involved in endoplasmic reticulum stress-induced apoptosis by enhancing DR5 expression in human carcinoma cells. J. Biol. Chem. 279, 45495-45502, doi:10.1074/jbc.M406933200 (2004).
45. Tischer, B. K., von Einem, J., Kaufer, B. & Osterrieder, N. Two-step red-mediated recombination for versatile high-efficiency markerless DNA manipulation in Escherichia coli. Biotechniques 40, 191-197 (2006).
46. Tischer, B. K., Smith, G. A. & Osterrieder, N. En passant mutagenesis: a two-step markerless red recombination system. Methods Mol. Biol. 634, 421-430, doi:10.1007/978-1-60761-652-8_30 (2010).
47. Wang, D. et al. The ULb′ region of the human cytomegalovirus genome confers an increased requirement for the viral protein kinase UL97. J. Virol. 87, 6359-6376, doi:10.1128/JVI.03477-12 (2013).
48. Li, G. et al. An epistatic relationship between the viral protein kinase UL97 and the UL133-UL138 latency locus during the human cytomegalovirus lytic cycle. J. Virol. 88, 6047-6060, doi:10.1128/JVI.00447-14 (2014).
49. Nguyen, C. C., Domma, A. J., Zhang, H. & Kamil, J. P. ER reorganization and intracellular retention of CD58 are functionally independent properties of the human cytomegalovirus ER resident glycoprotein UL148. J. Virol., doi:10.1128/JVI.01435-19 (2019).
50. Stanton, R. J. et al. Reconstruction of the complete human cytomegalovirus genome in a BAC reveals RL13 to be a potent inhibitor of replication. J. Clin. Invest. 120, 3191-3208, doi:10.1172/JC142955 (2010).
51. Jefferis, R., Lund, J. & Goodall, M. Recognition sites on human IgG for Fc gamma receptors: the role of glycosylation. Immunol. Lett. 44, 111-117, doi:10.1016/0165-2478(94)00201-2 (1995).
52. Radaev, S., Motyka, S., Fridman, W. H., Sautes-Fridman, C. & Sun, P. D. The structure of a human type III Fcgamma receptor in complex with Fc. J. Biol. Chem. 276, 16469-16477, doi:10.1074/jbc.M100350200 (2001).
53. Murrell, I. et al. The pentameric complex drives immunologically covert cell-cell transmission of wild-type human cytomegalovirus. Proc. Natl. Acad. Sci. U.S.A. 114, 6104-6109, doi:10.1073/pnas.1704809114 (2017).
54. Kim, J. A. et al. A novel electroporation method using a capillary and wire-type electrode. Biosens. Bioelectron. 23, 1353-1360, doi:10.1016/j.bios.2007.12.009 (2008).
55. Sanson, K. R. et al. Optimized libraries for CRISPR-Cas9 genetic screens with multiple modalities. Nat Commun 9, 5416, doi:10.1038/s41467-018-07901-8 (2018).
56. Schultz, E. P., Lanchy, J. M., Ellerbeck, E. E. & Ryckman, B. J. Scanning Mutagenesis of Human Cytomegalovirus Glycoprotein gH/gL. J. Virol. 90, 2294-2305, doi:10.1128/JVI.01875-15 (2015).
57. Vanarsdall, A. L., Ryckman, B. J., Chase, M. C. & Johnson, D. C. Human cytomegalovirus glycoproteins gB and gH/gL mediate epithelial cell-cell fusion when expressed either in cis or in trans. J. Virol. 82, 11837-11850, doi:10.1128/JVI.01623-08 (2008).
58. Turner, A., Bruun, B., Minson, T. & Browne, H. Glycoproteins gB, gD, and gHgL of herpes simplex virus type 1 are necessary and sufficient to mediate membrane fusion in a Cos cell transfection system. J Virol 72, 873-875 (1998).
59. Tang, J., Frascaroli, G., Lebbink, R. J., Ostermann, E. & Brune, W. Human cytomegalovirus glycoprotein B variants affect viral entry, cell fusion, and genome stability. Proc. Natl. Acad. Sci. U.S.A. 116, 18021-18030, doi:10.1073/pnas.1907447116 (2019).
60. Zhang, H. et al. Adenovirus-mediated artificial MicroRNAs targeting matrix or nucleoprotein genes protect mice against lethal influenza virus challenge. Gene Ther. 22, 653-662, doi:10.1038/gt.2015.31 (2015).
61. Lopez-Balderas, N. et al. In vitro cell fusion between CD4(+) and HIV-1 Env(+) T cells generates a diversity of syncytia varying in total number, size and cellular content. Virus Res. 123, 138-146, doi:10.1016/j.virusres.2006.08.009 (2007).
62. Schneider, W. M., Chevillotte, M. D. & Rice, C. M. Interferon-stimulated genes: a complex web of host defenses. Annu. Rev. Immunol. 32, 513-545, doi:10.1146/annurev-immunol-032713-120231 (2014).
63. Kumar-Sinha, C., Varambally, S., Sreekumar, A. & Chinnaiyan, A. M. Molecular cross-talk between the TRAIL and interferon signaling pathways. J. Biol. Chem. 277, 575-585, doi:10.1074/jbc.M107795200 (2002).
64. Toomey, N. L. et al. Induction of a TRAIL-mediated suicide program by interferon alpha in primary effusion lymphoma. Oncogene 20, 7029-7040, doi:10.1038/sj.onc.1204895 (2001).
65. Shigeno, M. et al. Interferon-alpha sensitizes human hepatoma cells to TRAIL-induced apoptosis through DR5 upregulation and NF-kappa B inactivation. Oncogene 22, 1653-1662, doi:10.1038/sj.onc.1206139 (2003).
66. Meng, R. D. & El-Deiry, W. S. p53-independent upregulation of KILLER/DR5 TRAIL receptor expression by glucocorticoids and interferon-gamma. Exp. Cell Res. 262, 154-169, doi:10.1006/excr.2000.5073 (2001).
67. Reis, C. R., Chen, P. H., Bendris, N. & Schmid, S. L. TRAIL-death receptor endocytosis and apoptosis are selectively regulated by dynamin-1 activation. Proc. Natl. Acad. Sci. U.S.A. 114, 504-509, doi:10.1073/pnas.1615072114 (2017).
68. Austin, C. D. et al. Death-receptor activation halts clathrin-dependent endocytosis. Proc. Natl. Acad. Sci. U.S.A. 103, 10283-10288, doi:10.1073/pnas.0604044103 (2006).

### REFERENCES FOR EXAMPLE 5

1. Heldwein E E. 2016. gH/gL supercomplexes at early stages of herpesvirus entry. Curr Opin Virol 18:1-8.
2. Chandramouli S, Malito E, Nguyen T, Luisi K, Donnarumma D, Xing Y, Norais N, Yu D, Carfi A. 2017. Structural basis for potent antibody-mediated neutralization of human cytomegalovirus. Sci Immunol 2.
3. Ciferri C, Chandramouli S, Donnarumma D, Nikitin P A, Cianfrocco M A, Gerrein R, Feire A L, Barnett S W, Lilja A E, Rappuoli R, Norais N, Settembre E C, Carfi A. 2015. Structural and biochemical studies of HCMV gH/gL/gO and Pentamer reveal mutually exclusive cell entry complexes. Proc Natl Acad Sci USA doi:10.1073/pnas.1424818112.
4. Wang D, Shenk T. 2005. Human cytomegalovirus virion protein complex required for epithelial and endothelial cell tropism. Proc Natl Acad Sci USA 102:18153-8.
5. Kabanova A, Marcandalli J, Zhou T, Bianchi S, Baxa U, Tsybovsky Y, Lilleri D, Silacci-Fregni C, Foglierini M, Fernandez-Rodriguez B M, Druz A, Zhang B, Geiger R, Pagani M, Sallusto F, Kwong P D, Corti D, Lanzavecchia A, Perez L. 2016. Platelet-derived growth factor-alpha receptor is the cellular receptor for human cytomegalovirus gHgLgO trimer. Nat Microbiol 1:16082.
6. Wu K, Oberstein A, Wang W, Shenk T. 2018. Role of PDGF receptor-alpha during human cytomegalovirus entry into fibroblasts. Proc Natl Acad Sci USA doi:10.1073/pnas.1806305115.
7. Wu Y, Prager A, Boos S, Resch M, Brizic I, Mach M, Wildner S, Scrivano L, Adler B. 2017. Human cytomegalovirus glycoprotein complex gH/gL/gO uses PDGFR-alpha as a key for entry. PLoS Pathog 13:e1006281.
8. Martinez-Martin N, Marcandalli J, Huang C S, Arthur C P, Perotti M, Foglierini M, Ho H, Dosey A M, Shriver S, Payandeh J, Leitner A, Lanzavecchia A, Perez L, Ciferri C. 2018. An Unbiased Screen for Human Cytomegalovirus Identifies Neuropilin-2 as a Central Viral Receptor. Cell 174:1158-1171 e19.
9. E X, Meraner P, Lu P, Perreira J M, Aker A M, McDougall W M, Zhuge R, Chan G C, Gerstein R M, Caposio P, Yurochko A D, Brass A L, Kowalik T F. 2019. OR14I1 is a receptor for the human cytomegalovirus pentameric complex and defines viral epithelial cell tropism. Proc Natl Acad Sci USA 116:7043-7052.
10. Vanarsdall A L, Pritchard S R, Wisner T W, Liu J, Jardetzky T S, Johnson D C. 2018. CD147 Promotes Entry of Pentamer-Expressing Human Cytomegalovirus into Epithelial and Endothelial Cells. MBio 9:e00781-18.
11. Jiang X J, Adler B, Sampaio K L, Digel M, Jahn G, Ettischer N, Stierhof Y D, Scrivano L, Koszinowski U, Mach M, Sinzger C. 2008. UL74 of human cytomegalovirus contributes to virus release by promoting secondary envelopment of virions. J Virol 82:2802-12.
12. Wille P T, Knoche A J, Nelson J A, Jarvis M A, Johnson D C. 2010. A human cytomegalovirus gO-null mutant fails to incorporate gH/gL into the virion envelope and is unable to enter fibroblasts and epithelial and endothelial cells. J Virol 84:2585-96.
13. Zhou M, Lanchy J M, Ryckman B J. 2015. Human Cytomegalovirus gH/gL/gO Promotes the Fusion Step of Entry into All Cell Types, whereas gH/gL/UL128-131 Broadens Virus Tropism through a Distinct Mechanism. J Virol 89:8999-9009.
14. Ryckman B J, Jarvis M A, Drummond D D, Nelson J A, Johnson D C. 2006. Human cytomegalovirus entry into epithelial and endothelial cells depends on genes UL128 to UL150 and occurs by endocytosis and low-pH fusion. J Virol 80:710-22.
15. Hahn G, Revello M G, Patrone M, Percivalle E, Campanini G, Sarasini A, Wagner M, Gallina A, Milanesi G, Koszinowski U, Baldanti F, Gerna G. 2004. Human cytomegalovirus UL131-128 genes are indispensable for virus growth in endothelial cells and virus transfer to leukocytes. J Virol 78:10023-33.
16. Calo S, Cortese M, Ciferri C, Bruno L, Gerrein R, Benucci B, Monda G, Gentile M, Kessler T, Uematsu Y, Maione D, Lilja A E, Carfi A, Merola M. 2016. The Human Cytomegalovirus UL116 Gene Encodes an Envelope Glycoprotein Forming a Complex with gH Independently from gL. J Virol 90:4926-38.
17. Siddiquey M N A, Amendola D, Vezzani G, Yu D, Maione D, Merola M, Kamil J. 2019. The HCMV Glycoprotein L Analog UL116 Interacts With Viral ER Resident Glycoprotein UL148 and Promotes Virion Incorporation of gH/gL Complexes. 44th Annual International Herpesvirus Workshop, Knoxville, TN:Oral Abstract 5.28.
18. Caposio P, van den Worm S, Crawford L, Perez W, Kreklywich C, Gilbride R M, Hughes C M, Ventura A B, Ratts R, Marshall E E, Malouli D, Axthelm M K, Streblow D, Nelson J A, Picker L J, Hansen S G, Fruh K. 2019. Characterization of a live-attenuated HCMV-based vaccine platform. Sci Rep 9:19236.
19. Li G, Nguyen C C, Ryckman B J, Britt W J, Kamil J P. 2015. A viral regulator of glycoprotein complexes contributes to human cytomegalovirus cell tropism. Proc Natl Acad Sci USA 112:4471-6.
20. Siddiquey M N A, Zhang H, Nguyen C C, Domma A J, Kamil J P. 2018. The Human Cytomegalovirus Endoplasmic Reticulum-Resident Glycoprotein UL148 Activates the Unfolded Protein Response. J Virol 92.
21. Zhang H, Read C, Nguyen C C, Siddiquey M N A, Shang C, Hall C M, von Einem J, Kamil J P. 2019. The Human Cytomegalovirus Nonstructural Glycoprotein UL148 Reorganizes the Endoplasmic Reticulum. mBio 10.
22. Sinzger C, Hahn G, Digel M, Katona R, Sampaio K L, Messerle M, Hengel H, Koszinowski U, Brune W, Adler B. 2008. Cloning and sequencing of a highly productive, endotheliotropic virus strain derived from human cytomegalovirus TB40/E. J Gen Virol 89:359-68.
23. Tomasec P, Wang E C, Davison A J, Vojtesek B, Armstrong M, Griffin C, McSharry B P, Morris R J, Llewellyn-Lacey S, Rickards C, Nomoto A, Sinzger C, Wilkinson G W. 2005. Downregulation of natural killer cell-activating ligand CD155 by human cytomegalovirus UL141. Nat Immunol 6:181-8.
24. Smith W, Tomasec P, Aicheler R, Loewendorf A, Nemcovicova I, Wang E C, Stanton R J, Macauley M, Norris P, Willen L, Ruckova E, Nomoto A, Schneider P, Hahn G, Zajonc D M, Ware C F, Wilkinson G W, Benedict C A. 2013. Human cytomegalovirus glycoprotein UL141 targets the TRAIL death receptors to thwart host innate antiviral defenses. Cell Host Microbe 13:324-35.
25. Sanchez V, Greis K D, Sztul E, Britt W J. 2000. Accumulation of virion tegument and envelope proteins in a stable cytoplasmic compartment during human cytomegalovirus replication: characterization of a potential site of virus assembly. J Virol 74:975-86.
26. Nobre L V, Nightingale K, Ravenhill B J, Antrobus R, Soday L, Nichols J, Davies J A, Seirafian S, Wang E C, Davison A J, Wilkinson G W, Stanton R J, Huttlin E L, Weekes M P. 2019. Human cytomegalovirus interactome analysis identifies degradation hubs, domain associations and viral protein functions. Elife 8.
27. Prod'homme V, Sugrue D M, Stanton R J, Nomoto A, Davies J, Rickards C R, Cochrane D, Moore M, Wilkinson G W, Tomasec P. 2010. Human cytomegalovirus UL141 promotes efficient downregulation of the natural killer cell activating ligand CD112. J Gen Virol 91:2034-9.
28. Wang D, Shenk T. 2005. Human cytomegalovirus UL131 open reading frame is required for epithelial cell tropism. J Virol 79:10330-8.
29. Sedger L M, Shows D M, Blanton R A, Peschon J J, Goodwin R G, Cosman D, Wiley SR. 1999. IFN-gamma mediates a novel antiviral activity through dynamic modulation of TRAIL and TRAIL receptor expression. J Immunol 163:920-6.
30. Shigeno M, Nakao K, Ichikawa T, Suzuki K, Kawakami A, Abiru S, Miyazoe S, Nakagawa Y, Ishikawa H, Hamasaki K, Nakata K, Ishii N, Eguchi K. 2003. Interferon-alpha sensitizes human hepatoma cells to TRAIL-induced apoptosis through DR5 upregulation and NF-kappa B inactivation. Oncogene 22:1653-62.
31. Kayagaki N, Yamaguchi N, Nakayama M, Eto H, Okumura K, Yagita H. 1999. Type I interferons (IFNs) regulate tumor necrosis factor-related apoptosis-inducing ligand (TRAIL) expression on human T cells: A novel mechanism for the antitumor effects of type I IFNs. J Exp Med 189:1451-60.
32. Toomey N L, Deyev V V, Wood C, Boise L H, Scott D, Liu L H, Cabral L, Podack E R, Barber G N, Harrington W J, Jr. 2001. Induction of a TRAIL-mediated suicide program by interferon alpha in primary effusion lymphoma. Oncogene 20:7029-40.
33. Umashankar M, Petrucelli A, Cicchini L, Caposio P, Kreklywich C N, Rak M, Bughio F, Goldman D C, Hamlin K L, Nelson J A, Fleming W H, Streblow D N, Goodrum F. A novel human cytomegalovirus locus modulates cell type-specific outcomes of infection. PLoS Pathog. 2011 December; 7(12):e1002444. doi: 10.1371/journal.ppat.1002444. Epub 2011 Dec. 29.
34. Fouts, Ashley E., et al. “Mechanism for neutralizing activity by the anti-CMV gH/gL monoclonal antibody MSL-109.” Proceedings of the National Academy of Sciences 111.22 (2014): 8209-8214.

What is claimed is:

1. A method of treating or preventing a cytomegalovirus (CMV) infection in a subject in need thereof, the method comprising administering to said subject a therapeutically or prophylactically effective amount of an agent that modulates a UL141/UL116/gH multimer.

2. The method of claim 1, wherein said agent modulates (a) binding of CMV to a host cell, (b) fusion of CMV with a host cell, (c) cell-to-cell spread of CMV or (d) multimerization of CMV proteins gH, UL116 and UL141.

3. The method of claim 1, wherein said agent (a) inhibits formation of the UL141/UL116/gH multimer (b) inhibits binding of UL141 to UL116/gH, or (c) inhibits binding of said multimer to a ligand thereof.

4. The method of claim 1, wherein said agent comprises UL141 or a fragment thereof.

5. The method of claim 1, wherein said agent comprises one or more of UL141, UL116, gH, gL, gO, UL128, UL130, UL131, or a fragment thereof.

6. The method of claim 1, wherein said agent comprises the UL141/UL116/gH multimer.

7. The method of claim 1, wherein said agent comprises a gH/gL/gO multimer.

8. The method of claim 1, wherein said agent comprises a gH/gL/UL128/UL130/UL131 multimer.

9. The method of claim 1, wherein said agent comprises an multimer comprising two or more of UL141, UL116, gH, gL, gO, UL128, UL130, UL131, or fragments thereof.

10. The method of claim 1, wherein said agent is a TRAIL receptor, CD155, CD112 or a fragment thereof.

11. The method of claim 10, wherein said TRAIL receptor is TRAIL receptor 1, TRAIL receptor 2, TRAIL receptor 3, TRAIL receptor 4, Osteoprotegrin (OPG) or a fragment thereof.

12. The method of claim 1, wherein said agent is a modified receptor protein.

13. The method of claim 12, wherein said modified receptor protein comprises an Fc domain.

14. The method of claim 1, wherein said agent is an antibody or antigen binding fragment thereof.

15. The method of claim 14, wherein said antibody or antigen binding fragment is a human antibody, a monoclonal antibody, a polyclonal antibody, a single chain antibody, Fab, Fab′, F(ab′)2, Fv or scFv.

16. The method of claim 14, wherein said antibody is an anti-UL141, anti-TRAIL receptor, anti-CD155, or anti-CD112 antibody.

17. The method of claim 14, wherein said antibody or antigen binding fragment thereof recognizes a multimer comprising UL141.

18. The method of claim 14, wherein said agent is a multispecific antibody that recognizes at least one epitope of UL141.

19. The method of claim 18, wherein said multispecific antibody is a bispecific antibody.

20. The method of claim 18, wherein said multispecific antibody is a trispecific antibody.

21. The method of claim 1, wherein said agent comprises a nucleic acid encoding UL141 or fragment thereof.

22. The method of claim 21, wherein said agent further comprises a nucleic acid encoding gH, gL, gO, UL116, UL128, UL130, UL131, or a fragment thereof.

23. The method of claim 1, wherein said agent comprises a nucleic acid encoding a TRAIL receptor, CD155, CD1121, or a fragment thereof.

24. The method of claim 23, wherein the TRAIL receptor is TRAIL receptor 1, TRAIL receptor 2, TRAIL receptor 3, TRAIL receptor 4 or OPG.

25. The method of claim 1, wherein said agent comprises a nucleic acid encoding a modified receptor protein.

26. The method of claim 25, wherein said modified receptor protein comprises an Fc domain.

27. The method of claim 1, wherein said agent comprises a nucleic acid encoding an antibody or antigen binding fragment.

28. The method of claim 27, wherein said antibody or antigen binding fragment is a human antibody, a monoclonal antibody, a polyclonal antibody, a single chain antibody, Fab, Fab′, F(ab′)2, Fv or scFv.

29. The method of claim 27, wherein said antibody or antigen binding fragment recognizes a multimer comprising UL141.

30. The method of claim 27, wherein said antibody is a multispecific antibody that recognizes at least one epitope of UL141.

31. The method of claim 30, wherein said multispecific antibody is a bispecific antibody.

32. The method of claim 30, wherein said multispecific antibody is a trispecific antibody.

33. The method of claim 21, wherein said nucleic acid further comprises a vector.

34. The method of claim 33, wherein said vector is a viral vector.

35. The method of claim 34, wherein said viral vector further comprises a recombinant virus.

36. The method of claim 1, wherein said agent comprises a small molecule, a peptide mimetic, an aptamer, or an inhibitory nucleic acid.

37. The method of claim 1, wherein said agent promotes an immune response in said subject.

38. The method of claim 37, said method further comprising administering an adjuvant to said subject.

39. The method of claim 1, wherein said agent further comprises a pharmaceutically acceptable excipient.

40. The method of claim 1, wherein CMV is human CMV (hCMV).

41. A method of preventing a cytomegalovirus (CMV) infection in a subject in need thereof, the method comprising administering to said subject a prophylactically effective amount of an agent comprising UL141 or a fragment thereof.

42. The method of claim 41, wherein said agent further comprises gH or a fragment thereof.

43. The method of claim 42, wherein said UL141 is non-covalently bound to gH.

44. The method of claim 42, wherein said UL141 is covalently bound to gH.

45. The method of claim 41, wherein said agent further comprises UL116.

46. The method of claim 45, wherein UL141 thereof is non-covalently bound to UL116.

47. The method of claim 45, wherein UL141 is covalently bound to UL116.

48. The method of claim 45, further comprising gH and UL116 or fragments thereof.

49. The method of claim 48, wherein said UL141, gH and UL116 are non-covalently bound.

50. The method of claim 48, wherein said UL141, gH and UL116 are covalently bound.

51. A vaccine composition comprising UL141 or a fragment thereof and a pharmaceutically acceptable excipient.

52. The vaccine composition of claim 51, further comprising gH or a fragment thereof.

53. The vaccine composition of claim 51, further comprising UL116 or a fragment thereof.

54. A method for screening agents that inhibit cytomegalovirus (CMV) host cell entry, fusion of CMV with a host cell, or cell-to-cell spread of CMV, comprising administering a test agent to a CMV-receptive cell, administering CMV to said CMV-receptive cell, and determining whether said agent modulates a UL141/UL116/gH multimer.

55. A composition comprising an agent coupled to a diagnostic agent, wherein said agent binds a UL141/UL116/gH multimer.

56. The composition of claim 55, wherein the diagnostic agent comprises a metal chelator bound to a metal ion, a small molecule, an antibody or functional fragment, a radioisotope, an enzyme, an oligonucleotide, an organic or inorganic nanoparticle, a chelator, a boron compound, a photoactive agent, a dye, fluorescent or luminescent substance, an enzyme, an enhancing agent, a radioactive substance, or a chelator.

57. A method of diagnosing cytomegalovirus (CMV) infection in a subject, comprising:
a) contacting a biological sample from said subject with the composition of claim 55; and
b) detecting binding of said agent to the UL141/UL116/gH multimer.

58. A composition comprising a nucleic acid encoding a protein having at least 80% sequence identity to the amino acid sequence of SEQ ID NO:15, 16, 17, 20, 22, 23, 26, 28, or 31.

59. A composition comprising one or more nucleic acids provided herein, or proteins encoded by said nucleic acids.


SEQUENCE LISTING (107 sequences)

SEQ ID NO: 1 - protein, 338 aa, human cytomegalovirus
MCRRESLRTLPWLFWVLLSCPQLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRRLPTYDSYPGVKKMKR

SEQ ID NO: 2 - protein, 314 aa, human cytomegalovirus
MKRRRRWRGWLLFPALCFCLLCEAVETNATTVTSTTAAAATTNTTVATTGTTTTSPNVTSTTSNTVTTPTTVSSVSNLTSSTTSIPISTSTVSGTRNTGNNNTTTIGTNATSPSPSVSILTTVTPAATSTISVDGVVTASDYTPTFDDLENITTTRAPTRPPAQDLCSHNLSIILYEEESQSSVDIAVDEEEPELEDDDEYDELWFPLYFEAECNRNYTLHVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQT

SEQ ID NO: 3 - protein, 742 aa, human cytomegalovirus
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 4 - protein, 906 aa, human cytomegalovirus
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 5 - protein, 278 aa, human cytomegalovirus
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 6 - protein, 472 aa, human cytomegalovirus
MGKKEMIMVKGIPKIMLLISITFLLLSLINCNVLVNSRGTRRSWPYTVLSYRGKEILKKQKEDILKRLMSTSSDGYRFLMYPSQQKFHAIVISMDKFPQDYILAGPIRNDSITHMWFDFYSTQLRKPAKYVYSEYNHTAHKITLRPPPCGTVPSMNCLSEMLNVSKRNDTGEKGCGNFTTFNPMFFNVPRWNTKLYIGSNKVNVDSQTIYFLGLTALLLRYAQRNCTRSFYLVNAMSRNLFRVPKYINGTKLKNTMRKLKRKQALVKEQPQKKNKKSQSTTTPYLSYTTSTAFNVTTNVTYSATAAVTRVATSTTGYRPDSNFMKSIMATQLRDLATWVYTTLRYRNEPFCKPDRNRTAVSEFMKNTHVLIRNETPYTIYGTLDMSSLYYNETMSVENETASDNNETTPTSPSTRFQRTFIDPLWDYLDSLLFLDKIRNFSLQLPAYGNLTPPEHRRAANLSTLNSLWWWSQ

SEQ ID NO: 7 - protein, 171 aa, human cytomegalovirus
MSPKDLTPFLTTLWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 8 - protein, 214 aa, human cytomegalovirus
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 9 - protein, 76 aa, human cytomegalovirus
MCMMSHNKAFFLSLQHAAVSGVAVCLSVRRGAGSVPAGNRGKKTIITEYRITGTRALARCPTKPVTSMWNSSWTSR

SEQ ID NO: 10 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 11 - DNA, 4154 bp, Homo sapiens
acttggacgcgcttgcggaggattgcgttgacgagactcttatttattgtcaccaacctgtggtggaatttgcagttgcacattggatctgattcgccccgccccgaatgacgcctgcccggaggcagtgaaagtacagccgcgccgccccaagtcagcctggacacataaatcagcacgcggccggagaaccccgcaatctctgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagtcccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtcagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcggggcagttcactctgtctcccaggctggagtgcaatggtgcaatcttggctcactatagccttgacctctcaggctcaagcgattctcccacctcagccatccaaatagctgggaccacaggtgtgcaccaccacgcccggctaattttttgtattttgtctagatataggggctctctatgttgctcagggtggtctcgaattcctggactcaagcagtctgcccacctcagactcccaaagcggtggaattagaggcgtgagcccccatgcttggccttacctttctacttttataattctgtatgttattattttatgaacatgaagaaactttagtaaatgtacttgtttacatagttatgtgaatagattagataaacataaaaggaggagacatacaatgggggaagaagaagaagtcccctgtaagatgtcactgtctgggttccagccctccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggctaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaa

SEQ ID NO: 12 - protein, 410 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAM

SEQ ID NO: 13 - DNA, 4067 bp, Homo sapiens
acttggacgcgcttgcggaggattgcgttgacgagactcttatttattgtcaccaacctgtggtggaatttgcagttgcacattggatctgattcgccccgccccgaatgacgcctgcccggaggcagtgaaagtacagccgcgccgccccaagtcagcctggacacataaatcagcacgcggccggagaaccccgcaatctctgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtcagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcggggcagttcactctgtctcccaggctggagtgcaatggtgcaatcttggctcactatagccttgacctctcaggctcaagcgattctcccacctcagccatccaaatagctgggaccacaggtgtgcaccaccacgcccggctaattttttgtattttgtctagatataggggctctctatgttgctcagggtggtctcgaattcctggactcaagcagtctgcccacctcagactcccaaagcggtggaattagaggcgtgagcccccatgcttggccttacctttctacttttataattctgtatgttattattttatgaacatgaagaaactttagtaaatgtacttgtttacatagttatgtgaatagattagataaacataaaaggaggagacatacaatgggggaagaagaagaagtcccctgtaagatgtcactgtctgggttccagccctccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggctaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaa

SEQ ID NO: 14 - protein, 51 aa, Homo sapiens
MEQRGQNAPAASGARKRHGPGPREARGARPGPRVPKTLVLVVAAVLLLVSA

SEQ ID NO: 15 - protein, 162 aa, Homo sapiens
ESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSG

SEQ ID NO: 16 - protein, 127 aa, Homo sapiens
QQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESG

SEQ ID NO: 17 - protein, 570 aa, Artificial Sequence
  Synthetic polypeptide
MAIIYLILLFTAVRGLEVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNGSVDLEVLFQGPKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 332 aa, Homo sapiens
MAIIYLILLFTAVRGLEVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRN

SEQ ID NO: 19 - DNA, 1713 bp, Artificial Sequence
  Synthetic polynucleotide
atggctatcatctacctcatcctcctgttcaccgctgtgcggggcctcgaggtcgtcgtgcaggcgcccacccaggtgcccggcttcttgggcgactccgtgacgctgccctgctacctacaggtgcccaacatggaggtgacgcatgtgtcacagctgacttgggcgcggcatggtgaatctggcagcatggccgtcttccaccaaacgcagggccccagctattcggagtccaaacggctggaattcgtggcagccagactgggcgcggagctgcggaatgcctcgctgaggatgttcgggttgcgcgtagaggatgaaggcaactacacctgcctgttcgtcacgttcccgcagggcagcaggagcgtggatatctggctccgagtgcttgccaagccccagaacacagctgaggttcagaaggtccagctcactggagagccagtgcccatggcccgctgcgtctccacagggggtcgcccgccagcccaaatcacctggcactcagacctgggcgggatgcccaatacgagccaggtgccagggttcctgtctggcacagtcactgtcaccagcctctggatattggtgccctcaagccaggtggacggcaagaatgtgacctgcaaggtggagcacgagagctttgagaagcctcagctgctgactgtgaacctcaccgtgtactaccccccagaggtatccatctctggctatgataacaactggtaccttggccagaatgaggccaccctgacctgcgatgctcgcagcaacccagagcccacaggctataattggagcacgaccatgggtcccctgccaccctttgctgtggcccagggcgcccagctcctgatccgtcctgtggacaaaccaatcaacacaactttaatctgcaacgtcaccaatgccctaggagctcgccaggcagaactgaccgtccaggtcaaagagggacctcccagtgagcactcaggcatatcccgtaacggatccgtcgacctggaggtgctgttccaggggcccaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtgctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 20 - protein, 467 aa, Artificial Sequence
  Synthetic polypeptide
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 231 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVH

SEQ ID NO: 22 - DNA, 1404 bp, Artificial Sequence
  Synthetic polynucleotide
atggcgccaccaccagctagagtacatctaggtgcgttcctggcagtgactccgaatcccgggagcgcagcgagtgggacagaggcagccgcggccacacccagcaaagtgtggggctcttccgcggggaggattgaaccacgaggcgggggccgaggagcgctccctacctccatgggacagcacggacccagtgcccgggcccgggcagggcgcgccccaggacccaggccggcgcgggaagccagccctcggctccgggtccacaagaccttcaagtttgtcgtcgtcggggtcctgctgcaggtcgtacctagctcagctgcaaccatcaaacttcatgatcaatcaattggcacacagcaatgggaacatagccctttgggagagttgtgtccaccaggatctcatagatcagaacatcctggagcctgtaaccggtgcacagagggtgtgggttacaccaatgcttccaacaatttgtttgcttgcctcccatgtacagcttgtaaatcagatgaagaagagagaagtccctgcaccacgaccaggaacacagcatgtcagtgcaaaccaggaactttccggaatgacaattctgctgagatgtgccggaagtgcagcagagggtgccccagagggatggtcaaggtcaaggattgtacgccctggagtgacatcgagtgtgtccacaaagaatcaggcaatggacataatgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtgctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 23 - protein, 440 aa, Artificial Sequence
  Synthetic polypeptide
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEAPAVEETVTSSPGTPASPCSLSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 178 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIEC

SEQ ID NO: 25 - DNA, 1323 bp, Artificial Sequence
  Synthetic polynucleotide
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagccccagctgtggaggagacggtgacctccagcccagggactcctgcctctccctgttctctctcagtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtgctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 26 - protein, 468 aa, Artificial Sequence
  Synthetic polypeptide
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNNSPGTPAPAAEETMNTSPGTPAPAAEETMTTSPGTPAPAAEETMTTSPGTPAPAAEETMTTSPGTPASSHYVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 235 aa, Homo sapiens
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNNSPGTPAPAAEETMNTSPGTPAPAAEETMTTSPGTPAPAAEETMTTSPGTPAPAAEETMTTSPGTP

SEQ ID NO: 28 - DNA, 1407 bp, Artificial Sequence
  Synthetic polynucleotide
atggcccggatccccaagaccctaaagttcgtcgtcgtcatcgtcgcggtcctgctgccagtcctagcttactctgccaccactgcccggcaggaggaagttccccagcagacagtggccccacagcaacagaggcacagcttcaagggggaggagtgtccagcaggatctcatagatcagaacatactggagcctgtaacccgtgcacagagggtgtggattacaccaacgcttccaacaatgaaccttcttgcttcccatgtacagtttgtaaatcagatcaaaaacataaaagttcctgcaccatgaccagagacacagtgtgtcagtgtaaagaaggcaccttccggaatgaaaactccccagagatgtgccggaagtgtagcaggtgccctagtggggaagtccaagtcagtaattgtacgtcctgggatgatatccagtgtgttgaagaatttggtgccaatgccactgtggaaaccccagctgctgaagagacaatgaacaacagcccggggactcctgccccagctgctgaagagacaatgaacaccagcccagggactcctgccccagctgctgaagagacaatgaccaccagcccggggactcctgccccagctgctgaagagacaatgaccaccagcccggggactcctgccccagctgctgaagagacaatgaccaccagcccggggactcctgcctcttctcattacgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtgctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 29 - protein, 439 aa, Artificial Sequence
  Synthetic polypeptide
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 211 aa, Homo sapiens
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYH

SEQ ID NO: 31 - DNA, 1320 bp, Artificial Sequence
  Synthetic polynucleotide
atgggactttggggacaaagcgtcccgaccgcctcgagcgctcgagcagggcgctatccaggagccaggacagcgtcgggaaccagaccatggctcctggaccccaagatccttaagttcgtcgtcttcatcgtcgcggttctgctgccggtccgggttgactctgccaccatcccccggcaggacgaagttccccagcagacagtggccccacagcaacagaggcgcagcctcaaggaggaggagtgtccagcaggatctcatagatcagaatatactggagcctgtaacccgtgcacagagggtgtggattacaccattgcttccaacaatttgccttcttgcctgctatgtacagtttgtaaatcaggtcaaacaaataaaagttcctgtaccacgaccagagacaccgtgtgtcagtgtgaaaaaggaagcttccaggataaaaactcccctgagatgtgccggacgtgtagaacagggtgtcccagagggatggtcaaggtcagtaattgtacgccccggagtgacatcaagtgcaaaaatgaatcagctgccagttccactgggaaaaccccagcagcggaggagacagtgaccaccatcctggggatgcttgcctctccctatcacgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtgctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 32 - protein, 479 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPRASPRDVGPLVWGAVGGTLLVLLLLAGGSLAFILLRVRRRRKSPGGAGGGASGDGGFYDPKAQVLGNGDPVFWTPVVPGPMEPDGKDEEEEEEEEKAEKGLMLPPPPALEDDMESQLDGSLISRRAVYV

SEQ ID NO: 33 - DNA, 1440 bp, Homo sapiens
atggcccgggccgctgccctcctgccgtcgagatcgccgccgacgccgctgctgtggccgctgctgctgctgctgctcctggaaaccggagcccaggatgtgcgagttcaagtgctacccgaggtgcgaggccagctcgggggcaccgtggagctgccgtgccacctgctgccacctgttcctggactgtacatctccctggtgacctggcagcgcccagatgcacctgcgaaccaccagaatgtggccgccttccaccctaagatgggtcccagcttccccagcccgaagcctggcagcgagcggctgtccttcgtctctgccaagcagagcactgggcaagacacagaggcagagctccaggacgccacgctggccctccacgggctcacggtggaggacgagggcaactacacttgcgagtttgccaccttccccaaggggtccgtccgagggatgacctggctcagagtcatagccaagcccaagaaccaagctgaggcccagaaggtcacgttcagccaggaccctacgacagtggccctctgcatctccaaagagggccgcccacctgcccggatctcctggctctcatccctggactgggaagccaaagagactcaggtgtcagggaccctggccggaactgtcactgtcaccagccgcttcaccttggtgccctcgggccgagcagatggtgtcacggtcacctgcaaagtggagcatgagagcttcgaggaaccagccctgatacctgtgaccctctctgtacgctaccctcctgaagtgtccatctccggctatgatgacaactggtacctcggccgtactgatgccaccctgagctgtgacgtccgcagcaacccagagcccacgggctatgactggagcacgacctcaggcaccttcccgacctccgcagtggcccagggctcccagctggtcatccacgcagtggacagtctgttcaataccaccttcgtctgcacagtcaccaatgccgtgggcatgggccgcgctgagcaggtcatctttgtccgagaaacccccagggcctcgccccgagatgtgggcccgctggtgtggggggccgtgggggggacactgctggtgctgctgcttctggctggggggtccttggccttcatcctgctgagggtgaggaggaggaggaagagccctggaggagcaggaggaggagccagtggcgacgggggattctacgatccgaaagctcaggtgttgggaaatggggaccccgtcttctggacaccagtagtccctggtcccatggaaccagatggcaaggatgaggaggaggaggaggaggaagagaaggcagagaaaggcctcatgttgcctccacccccagcactcgaggatgacatggagtcccagctggacggctccctcatctcacggcgggcagtttatgtgtga

SEQ ID NO: 34 - protein, 743 aa, human cytomegalovirus
MRPGLPSYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPTTVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 35 - protein, 278 aa, human cytomegalovirus
MCRRPDCGFSFSPGPVVLLWCCLLLPIVSSVAVSVAPTAAEKVPAECPELTRRCLLGEVFQGDKYESWLRPLVNVTGRNGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 36 - protein, 278 aa, human cytomegalovirus
MCRRPDCGFSFSPGPVILLWCCLLLSIVSSAAVSVAPTAAEKVPAECPELTRRCLLGEVFQGDKYESWLRPLVNVTGRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 37 - protein, 329 aa, Artificial Sequence
  Synthetic polypeptide
MKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQTGGSGGSEQKLISEEDL

SEQ ID NO: 38 - protein, 338 aa, human cytomegalovirus
MCRRESLRTLPWLFWVLLSCPRLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRRLPTYDSYPGVKKMKR

SEQ ID NO: 39 - protein, 323 aa, Artificial Sequence
  Synthetic polypeptide
MKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQTEQKLISEEDL

SEQ ID NO: 40 - protein, 338 aa, Artificial Sequence; identical to SEQ ID NO: 38
  Synthetic polypeptide

SEQ ID NO: 41 - protein, 346 aa, Artificial Sequence
  Synthetic polypeptide
MCRRESLRTLPWLFWVLLSCPRLLEYSSSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGIAAFLGFWSIFTVCFLCYLCYLQCCGRWCPTPGRGRRGGEGYRRLPTYDSYPGVKKMKRDYKDDDDK

SEQ ID NO: 42 - protein, 346 aa, Artificial Sequence; identical to SEQ ID NO: 41
  Synthetic polypeptide

SEQ ID NO: 43 - protein, 289 aa, Artificial Sequence
  Synthetic polypeptide
MKWVTFISLLFLFSSAYSSFPFATADIAEKMWAENYETTSPAPVLVAEGEQVTIPCTVMTHSWPMVSIRARFCRSHDGSDELILDAVKGHRLMNGLQYRLPYATWNFSQLHLGQIFSLTFNVSTDTAGMYECVLRNYSHGLIMQRFVILTQLETLSRPDEPCCTPALGRYSLGDQIWSPTPWRLRNHDCGMYRGFQRNYFYIGRADAEDCWKPACPDEEPDRCWTVIQRYRLPGDCYRSQPHPPKFLPVTPAPPADIDTGMSPWATRGGSGGGSKETAAAKFERQHMDS

SEQ ID NO: 44 - protein, 757 aa, Artificial Sequence
  Synthetic polypeptide
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTCGGSGGSDYKDDDDK

SEQ ID NO: 45 - protein, 723 aa, Artificial Sequence
  Synthetic polypeptide
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDGGSGGSDYKDDDDK

SEQ ID NO: 46 - protein, 1105 aa, Artificial Sequence
  Synthetic polypeptide
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDGGSGGSWSHPQFEKGGGSGGGSGGSAWSHPQFEKGSGATNFSLLKQAGDVEENPGPMKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQTGGSGGSEQKLISEEDLGSGHHHHHHET

SEQ ID NO: 47 - protein, 289 aa, Artificial Sequence; identical to SEQ ID NO: 43
  Synthetic polypeptide

SEQ ID NO: 48 - protein, 765 aa, Artificial Sequence
  Synthetic polypeptide
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDGGSGGSWSHPQFEKGGGSGGGSGGSAWSHPQFEKGSGATNFSLLKQAGDVEENPGP

SEQ ID NO: 49 - protein, 362 aa, Artificial Sequence
  Synthetic polypeptide
GSGATNFSLLKQAGDVEENPGPMKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQTGGSGGSEQKLISEEDLGSGHHHHHHET

SEQ ID NO: 50 - DNA, 12564 bp, Artificial Sequence
  Synthetic polynucleotide
  misc_feature (6767)..(6767): n is a, c, g, or t
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggggggggggggggggggggggggggggggggggcggagagggcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttttgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatctagataccctcgtaaaggtctagagctagccaccatgagacccgggctcccattctatctgactgtgtttgctgtttacctgctcagccaccttccatcacagcgatacggtgccgatgctgcgagtgaggctctggatccacacgcttttcacctgctgttgaacacgtatggccgccccatacgctttcttcgcgagaatactacacagtgcacctacaactctagcttgagaaactctaccgtggtaagggaaaatgccatctcctttaatttctttcagagctataaccagtactatgtcttccacatgcccagatgcttgttcgctggacccctggcggaacagtttctgaatcaagtagacttgacagaaaccctcgaaaggtaccagcagcggctgaatacgtatgctcttgtgagcaaggatctcgccagctaccgctccttctcacagcaactcaaagcccaggacagcctgggccaacagcctactaccgtgcccccaccgattgaccttagtattcctcatgtctggatgccacctcagaccaccccacatgattggaaaggtagccacaccacatctgggcttcataggccccacttcaaccagacgtgtattctgttcgatggacacgatctgctcttcagcaccgtgacaccatgcctgcatcaaggcttttacctgatggatgaactgagatacgtgaagattaccctgaccgaggattttttcgtggtgaccgtgtccatagacgatgacacacccatgctcctgatatttgggcacttgccaagggtgctgttcaaagccccgtaccaaagggataattttatactgagacagacagaaaaacacgagcttttggtgctggttaaaaaaacacagctgaacagacactcctaccttaaggattctgacttcttggacgctgcgctggacttcaattacctggatctgtccgctctgttgcgcaatagtttccacaggtacgcagtcgatgtactgaagtccggccgctgccagatgctggaccgcaggaccgtagaaatggcctttgcatatgcccttgccctgtttgcggcagctagacaggaggaagcaggtacggagatttccatccctcgggccctggacaggcaggccgcactgctgcagatccaggaattcatgatcacatgtttgtcccagacaccgcctcggacaactctgctgctctaccccacagctgttgaccttgccaagcgagccctttggacacccgaccagatcacagatatcacttctctggtccgcctcgtttatatcctttcaaaacagaatcagcaacacctcattccacagtgggcacttcgccagatcgcagactttgccttgcaactccacaagactcatctggcgtcctttttgtctgctttcgctcggcaagagctgtacttgatgggatctcttgtacattcaatgctggtgcatactactgaaaggcgcgagatttttattgtggagaccggcctgtgctctctggccgagctcagccatttcacacaactcctggctcatcctcaccatgagtatttgtcagatctctatacgccttgctccagtagtggccgcagggaccatagcttggaacggctgacacgcctgtttccggatgctaccgtgccagccaccgtgcccgcagctctgagtatactgtccaccatgcagccaagcacgctggagacctttcctgatctcttttgcctgcctctgggggagtctttctctgctctgactgtgtccgaacatgtgtcttatgttgttacgaatcagtacctcattaagggcatttcctaccctgtcagcacaacagttgtgggtcagtccttgatcattacccaaacggatagccagtccaaatgcgaactgacaaggaacatgcacacaactcatagcattactgccgcattgaacattagtctggaaaactgcgctttctgccagagcgcgctcctcgaatacgacgacacgcaaggcgtgatcaacatcatgtatatgcatgacagtgatgacgtgctcttcgcccttgacccttacaacgaggtggttgtatcaagtcctcgaactcactacctgatgctcctcaaaaatggtacagtcctcgaagtgaccgacggcggttccggcggttcttggtcccatcctcagttcgagaaaggtggaggtagcggaggggggtctggaggcagtgcgtggagccatcctcaatttgagaaaggctccggagcaacaaattttagcctcctgaaacaggctggcgatgtggaagagaatcctggaccaatgaaaagacggaggagatggcgaggttggcttctttttctcgcgttgtgtttctgccttttgtgtgaggctgtagaaactaacgcgaccacggtaacatctaccaccgctgctgcggccacaacaaatacgaccgtggccacgactggaactacgacgacgagtccgaatgtaacgtccactacatccaatacggtgattacccccacgacagtctctagcgtctccaatctgacatcaagtgcgacttccattcccatatctacttctaccgtttctgggactagaaacacccgaaacaataacactactaccatcggaactaatgttacctcaccatccccatcagttagtattcttaccaccgtcactccagcggccacgtctacaacatccaacaacggtgatgtgacgtccgactatacaccgaccttcgaccttgagaacatcacaaccacgagagcgccaactagaccccctgcccaagacctgtgcagtcataacctctcaattattttgtacgaggaagagtcccagtccagtgtagatattgcggtggacgaggaggagcccgagcttgaagacgacgacgaatatgacgaattgtggttcccactttattttgaagctgaatgcaatttgaattacactttgcagtacgtcaaccacagctgtgactattcagtacgacagtcctccgtatctttccccccttggagagacatagacagtgttacgttcgtccctcgaaacttgtccaattgctctgcacatggtctcgccgtgatagtagcgggaaaccagacctggtacgtcaatcctttttcacttgcccaccttctggacgccatctacaatgtactcggtatagaagacttgtcagcgaactttcggagacagttggccccctaccgccacacgctgatcgtcccacaaacaggtgggagcggcggctcagagcaaaagttgatcagcgaagaggatctcggaagcggccatcatcaccatcaccatgagacgtaagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgaaatcggatccgcgggtcaaagttggcgttttattattatagtcagntctagagatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcggaattgtgagcggataacaattggtaccgtcgcagtcttcggtctgaccaccgtagaacgcagatcgaattgggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatcgatctggccatacacttgagtgacaatgacatccactttgcctttctctccacaggtgtccactcccaggtccaaccgaattcgccaccatgaagtgggtcacctttatcagcctgttgtttttgttttccagtgcttattcttccttcccatttgcgaccgcagacattgctgaaaaaatgtgggcagagaactacgagacaacaagccctgctccagtgctcgtggccgaaggggagcaagttacgattccgtgtactgtgatgacgcacagttggcctatggtgagcataagagcgagattctgtagaagtcacgatgggtcagacgagcttatattggatgctgttaagggacatcgcctgatgaatggcttgcaataccgcctcccctacgcaacgtggaatttttcacaactccatcttggacagattttcagcttgacatttaatgtatcaacagacacggcaggaatgtacgagtgcgtactcaggaactacagtcatggccttataatgcaacgatttgtaatcctgacgcagctggagactttgagccggcctgatgagccgtgctgcacaccggctctgggccgatactccctgggcgaccagatttggtcacctacaccctggagactccgaaaccacgactgtggcatgtatagggggttccaacggaattatttttatataggacgagcagacgccgaagattgctggaaaccggcttgcccagacgaggagccagatcgatgctggacagttatacaacgatatagacttccaggtgattgctacaggtcacaaccccacccacctaaattcttgccggttactcctgctcctcctgcagatatcgatactggtatgtcaccctgggcgactagaggcgggtcagggggtggatcaaaagagacagcggcggctaaatttgaaaggcagcatatggacagctaggcactgattctagctgcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggatcctcgagccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagttaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 51 - protein, 289 aa, Artificial Sequence; identical to SEQ ID NO: 43
  Synthetic polypeptide

SEQ ID NO: 52 - protein, 286 aa, Artificial Sequence [sequence omitted]

SEQ ID NO: 53 - DNA, 5119 bp, Artificial Sequence
  Synthetic polynucleotide
  misc_feature (454)..(454): n is a, c, g, or t
ttcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagntctagagatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcggaattgtgagcggataacaattggtaccgtcgcagtcttcggtctgaccaccgtagaacgcagatcgaattgggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatcgatctggccatacacttgagtgacaatgacatccactttgcctttctctccacaggtgtccactcccaggtccaaccgaattcgccaccatgaagtgggtcacctttatcagcctgttgtttttgttttccagtgcttattcttccttcccatttgcgaccgcagacattgctgaaaaaatgtgggcagagaactacgagacaacaagccctgctccagtgctcgtggccgaaggggagcaagttacgattccgtgtactgtgatgacgcacagttggcctatggtgagcataagagcgagattctgtagaagtcacgatgggtcagacgagcttatattggatgctgttaagggacatcgcctgatgaatggcttgcaataccgcctcccctacgcaacgtggaatttttcacaactccatcttggacagattttcagcttgacatttaatgtatcaacagacacggcaggaatgtacgagtgcgtactcaggaactacagtcatggccttataatgcaacgatttgtaatcctgacgcagctggagactttgagccggcctgatgagccgtgctgcacaccggctctgggccgatactccctgggcgaccagatttggtcacctacaccctggagactccgaaaccacgactgtggcatgtatagggggttccaacggaattatttttatataggacgagcagacgccgaagattgctggaaaccggcttgcccagacgaggagccagatcgatgctggacagttatacaacgatatagacttccaggtgattgctacaggtcacaaccccacccacctaaattcttgccggttactcctgctcctcctgcagatatcgatactggtatgtcaccctgggcgactagaggcgggtcagggggtggatcaaaagagacagcggcggctaaatttgaaaggcagcatatggacagctagtttaaaccaagctgctagcaaggatccagcttgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatccgaagttcctattcttactagagtataggaacttcgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcactctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtata

SEQ ID NO: 54 - DNA, 5303 bp, Artificial Sequence
  Synthetic polynucleotide
  misc_feature (454)..(454): n is a, c, g, or t
ttcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagntctagagatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcggaattgtgagcggataacaattggtaccgtcgcagtcttcggtctgaccaccgtagaacgcagatcgaattgggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatcgatctggccatacacttgagtgacaatgacatccactttgcctttctctccacaggtgtccactcccaggtccaaccgaattctgcagataccatgaaaagacgaaggagatggagaggttggcttctttttctcgcgttgtgtttctgccttttgtgtgaggctgtagaaactaacgcgaccacggtaacatctaccaccgctgctgcggccacaacaaatacgaccgtggccacgactggaactacgacgacgagtccgaatgtaacgtccactacatccaatacggtgattacccccacgacagtctctagcgtctccaatctgacatcaagtgcgacttccattcccatatctacttctaccgtttctgggactagaaacacccgaaacaataacactactaccatcggaactaatgttacctcaccatccccatcagttagtattcttaccaccgtcactccagcggccacgtctacaacatccaacaacggtgatgtgacgtccgactatacaccgaccttcgaccttgagaacatcacaaccacgagagcgccaactagaccccctgcccaagacctgtgcagtcataacctctcaattattttgtacgaggaagagtcccagtccagtgtagatattgcggtggacgaggaggagcccgagcttgaagacgacgacgaatatgacgaattgtggttcccactttattttgaagctgaatgcaatttgaattacactttgcagtacgtcaaccacagctgtgactattcagtacgacagtcctccgtatctttccccccttggagagacatagacagtgttacgttcgtccctcgaaacttgtccaattgctctgcacatggtctcgccgtgatagtagcgggaaaccagacctggtacgtcaatcctttttcacttgcccaccttctggacgccatctacaatgtactcggtatagaagacttgtcagcgaactttcggagacagttggccccctaccgccacacgctgatcgtcccacaaacaggtgggagcggcggctcagagcaaaagttgatcagcgaagaggatctctgatccagcacagtggcggccgctcgaggtcacccattcgaaggtaagcctatccctaaccctctcctcggtctcgattctacgcgtaccggtcatcatcaccatcaccattgagtttgggagatctaagcttgagctcgtcgacttcgagcaacttgtttattgcagcttataatggttacaaataaagcaatagcatcacaaatttcacaaataaagcatttttttcactgcattctagttgtggtttgtccaaactcatcaatgtatcttatcatgtctggatcgtctagcatcgaagatccaataacttcgtatagcatacattatacgaagttataagtagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcactctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtata

SEQ ID NO: 55 - protein, 206 aa, Artificial Sequence [sequence omitted]

SEQ ID NO: 56 - protein, 264 aa, Artificial Sequence [sequence omitted]

SEQ ID NO: 57 - DNA, 8359 bp, Artificial Sequence
  Synthetic polynucleotide
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctagttaagcttggtaccgagctcggatccactagtccagtgtggtggaattctgcagataccatgagacccgggctcccattctatctgactgtgtttgctgtttacctgctcagccaccttccatcacagcgatacggtgccgatgctgcgagtgaggctctggatccacacgcttttcacctgctgttgaacacgtatggccgccccatacgctttcttcgcgagaatactacacagtgcacctacaactctagcttgagaaactctaccgtggtaagggaaaatgccatctcctttaatttctttcagagctataaccagtactatgtcttccacatgcccagatgcttgttcgctggacccctggcggaacagtttctgaatcaagtagacttgacagaaaccctcgaaaggtaccagcagcggctgaatacgtatgctcttgtgagcaaggatctcgccagctaccgctccttctcacagcaactcaaagcccaggacagcctgggccaacagcctactaccgtgcccccaccgattgaccttagtattcctcatgtctggatgccacctcagaccaccccacatgattggaaaggtagccacaccacatctgggcttcataggccccacttcaaccagacgtgtattctgttcgatggacacgatctgctcttcagcaccgtgacaccatgcctgcatcaaggcttttacctgatggatgaactgagatacgtgaagattaccctgaccgaggattttttcgtggtgaccgtgtccatagacgatgacacacccatgctcctgatatttgggcacttgccaagggtgctgttcaaagccccgtaccaaagggataattttatactgagacagacagaaaaacacgagcttttggtgctggttaaaaaaacacagctgaacagacactcctaccttaaggattctgacttcttggacgctgcgctggacttcaattacctggatctgtccgctctgttgcgcaatagtttccacaggtacgcagtcgatgtactgaagtccggccgctgccagatgctggaccgcaggaccgtagaaatggcctttgcatatgcccttgccctgtttgcggcagctagacaggaggaagcaggtacggagatttccatccctcgggccctggacaggcaggccgcactgctgcagatccaggaattcatgatcacatgtttgtcccagacaccgcctcggacaactctgctgctctaccccacagctgttgaccttgccaagcgagccctttggacacccgaccagatcacagatatcacttctctggtccgcctcgtttatatcctttcaaaacagaatcagcaacacctcattccacagtgggcacttcgccagatcgcagactttgccttgcaactccacaagactcatctggcgtcctttttgtctgctttcgctcggcaagagctgtacttgatgggatctcttgtacattcaatgctggtgcatactactgaaaggcgcgagatttttattgtggagaccggcctgtgctctctggccgagctcagccatttcacacaactcctggctcatcctcaccatgagtatttgtcagatctctatacgccttgctccagtagtggccgcagggaccatagcttggaacggctgacacgcctgtttccggatgctaccgtgccagccaccgtgcccgcagctctgagtatactgtccaccatgcagccaagcacgctggagacctttcctgatctcttttgcctgcctctgggggagtctttctctgctctgactgtgtccgaacatgtgtcttatgttgttacgaatcagtacctcattaagggcatttcctaccctgtcagcacaacagttgtgggtcagtccttgatcattacccaaacggatagccagtccaaatgcgaactgacaaggaacatgcacacaactcatagcattactgccgcattgaacattagtctggaaaactgcgctttctgccagagcgcgctcctcgaatacgacgacacgcaaggcgtgatcaacatcatgtatatgcatgacagtgatgacgtgctcttcgcccttgacccttacaacgaggtggttgtatcaagtcctcgaactcactacctgatgctcctcaaaaatggtacagtcctcgaagtgaccgacggaggttctggaggctctgactacaaggatgacgacgataagtgaatccagcacagtggcggccgctcgaggtcacccattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 58 - protein, 206 aa, Artificial Sequence [sequence omitted]

SEQ ID NO: 59 - protein, 264 aa, Artificial Sequence [sequence omitted]

SEQ ID NO: 60 - protein, 286 aa, Artificial Sequence
  Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 61 - DNA, 8461 bp, Artificial Sequence
  Synthetic polynucleotide
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctagttaagcttggtaccgagctcggatccactagtccagtgtggtggaattctgcagataccatgagacccgggctcccattctatctgactgtgtttgctgtttacctgctcagccaccttccatcacagcgatacggtgccgatgctgcgagtgaggctctggatccacacgcttttcacctgctgttgaacacgtatggccgccccatacgctttcttcgcgagaatactacacagtgcacctacaactctagcttgagaaactctaccgtggtaagggaaaatgccatctcctttaatttctttcagagctataaccagtactatgtcttccacatgcccagatgcttgttcgctggacccctggcggaacagtttctgaatcaagtagacttgacagaaaccctcgaaaggtaccagcagcggctgaatacgtatgctcttgtgagcaaggatctcgccagctaccgctccttctcacagcaactcaaagcccaggacagcctgggccaacagcctactaccgtgcccccaccgattgaccttagtattcctcatgtctggatgccacctcagaccaccccacatgattggaaaggtagccacaccacatctgggcttcataggccccacttcaaccagacgtgtattctgttcgatggacacgatctgctcttcagcaccgtgacaccatgcctgcatcaaggcttttacctgatggatgaactgagatacgtgaagattaccctgaccgaggattttttcgtggtgaccgtgtccatagacgatgacacacccatgctcctgatatttgggcacttgccaagggtgctgttcaaagccccgtaccaaagggataattttatactgagacagacagaaaaacacgagcttttggtgctggttaaaaaaacacagctgaacagacactcctaccttaaggattctgacttcttggacgctgcgctggacttcaattacctggatctgtccgctctgttgcgcaatagtttccacaggtacgcagtcgatgtactgaagtccggccgctgccagatgctggaccgcaggaccgtagaaatggcctttgcatatgcccttgccctgtttgcggcagctagacaggaggaagcaggtacggagatttccatccctcgggccctggacaggcaggccgcactgctgcagatccaggaattcatgatcacatgtttgtcccagacaccgcctcggacaactctgctgctctaccccacagctgttgaccttgccaagcgagccctttggacacccgaccagatcacagatatcacttctctggtccgcctcgtttatatcctttcaaaacagaatcagcaacacctcattccacagtgggcacttcgccagatcgcagactttgccttgcaactccacaagactcatctggcgtcctttttgtctgctttcgctcggcaagagctgtacttgatgggatctcttgtacattcaatgctggtgcatactactgaaaggcgcgagatttttattgtggagaccggcctgtgctctctggccgagctcagccatttcacacaactcctggctcatcctcaccatgagtatttgtcagatctctatacgccttgctccagtagtggccgcagggaccatagcttggaacggctgacacgcctgtttccggatgctaccgtgccagccaccgtgcccgcagctctgagtatactgtccaccatgcagccaagcacgctggagacctttcctgatctcttttgcctgcctctgggggagtctttctctgctctgactgtgtccgaacatgtgtcttatgttgttacgaatcagtacctcattaagggcatttcctaccctgtcagcacaacagttgtgggtcagtccttgatcattacccaaacggatagccagtccaaatgcgaactgacaaggaacatgcacacaactcatagcattactgccgcattgaacattagtctggaaaactgcgctttctgccagagcgcgctcctcgaatacgacgacacgcaaggcgtgatcaacatcatgtatatgcatgacagtgatgacgtgctcttcgcccttgacccttacaacgaggtggttgtatcaagtcctcgaactcactacctgatgctcctcaaaaatggtacagtcctcgaagtgaccgacgttgtggtagacgcgaccgactcccgcctgctcatgatgtcagtgtatgccctgtctgccattatcggcatatatctgctctatcgaatgctgaaaacttgcggaggttctggaggctctgactataaggacgatgacgacaaatgaatccagcacagtggcggccgctcgaggtcacccattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 62 - DNA, 7176 bp, Artificial Sequence
  Synthetic polynucleotide
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgcctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctagttaagcttggtaccgagctcggatccactagtccagtgtggtggaattctgcagataccatgaaaagacgaaggagatggagaggttggcttctttttctcgcgttgtgtttctgccttttgtgtgaggctgtagaaactaacgcgaccacggtaacatctaccaccgctgctgcggccacaacaaatacgaccgtggccacgactggaactacgacgacgagtccgaatgtaacgtccactacatccaatacggtgattacccccacgacagtctctagcgtctccaatctgacatcaagtgcgacttccattcccatatctacttctaccgtttctgggactagaaacacccgaaacaataacactactaccatcggaactaatgttacctcaccatccccatcagttagtattcttaccaccgtcactccagcggccacgtctacaacatccaacaacggtgatgtgacgtccgactatacaccgaccttcgaccttgagaacatcacaaccacgagagcgccaactagaccccctgcccaagacctgtgcagtcataacctctcaattattttgtacgaggaagagtcccagtccagtgtagatattgcggtggacgaggaggagcccgagcttgaagacgacgacgaatatgacgaattgtggttcccactttattttgaagctgaatgcaatttgaattacactttgcagtacgtcaaccacagctgtgactattcagtacgacagtcctccgtatctttccccccttggagagacatagacagtgttacgttcgtccctcgaaacttgtccaattgctctgcacatggtctcgccgtgatagtagcgggaaaccagacctggtacgtcaatcctttttcacttgcccaccttctggacgccatctacaatgtactcggtatagaagacttgtcagcgaactttcggagacagttggccccctaccgccacacgctgatcgtcccacaaacaggtgggagcggcggctcagagcaaaagttgatcagcgaagaggatctctgatccagcacagtggcggccgctcgaggtcacccattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 63 - DNA, 1132 bp, Artificial Sequence
  Synthetic polynucleotide
gacttacgatagttaccccggtgttaaaaagatgaagagggactacaaggatgacgacgataagtgagaacatagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggctgaagagggactacaaggatgacgacgataagtgagaacacgcataaaataaaaaaataacatgttaaaaaa

SEQ ID NO: 64 - protein, 191 aa, Artificial Sequence
  Synthetic polypeptide
MTPAQTNATTTVHPHDTKNGSGGSALPTLVVFGFIITLLFFLFMLYFWNNDVFRKLLRCAWIQRCCDRFDAWQDEVIYRRPSRRSQSDDESRTNSVSSYVLLSPASDGGFDNPALTEAVDSVDDWATTSVFYATSDETADTERRDSQQLLIELPPEPLPPDVVAAMQKAVKRAVQNALRHSHDSWQLHQTL

SEQ ID NO: 65 - protein, 70 aa, Artificial Sequence
  Synthetic polypeptide
MCRRESLRTLPWLFWVLLSCPRLLEYSSSSFPFATADIAEKMWAENYETTSPAPVLVAEGEQVIPSPARS

SEQ ID NO: 66 - protein, 305 aa, Artificial Sequence
  Synthetic polypeptide
MRIEWACWLFGYFVSSVGSERSLSYRYHLESNSSANVVCNGNISVFVNGTLGVRYNITVGISSSLLIGHLTIQTLESWFTSWVQNKSYSKQPLSTTETLYNIDSENIHRVSQYFHTRWIKSLQENHTCDLTNSTPTYTYQANVNNTNYLTLTSSGWQDRLNYTAINSTHFNLTESNITSIHKYLNTTCIERLRNYTLKSVYTTTVPQNVTTPQHTTTTLYTPPNAITIQDTTQSHTVQTPSFNDTHNVTEHTLNINYVLSQKTNNTTSPWVYAIPMGATATIGASLYIGKHFTPVRFVYEVWRGQ

SEQ ID NO: 67 - DNA, 3933 bp, Artificial Sequence
  Synthetic polynucleotide
atgaccccagctcagactaacgccactaccaccgtgcacccgcacgacacaaaaaacggcagcggcggtagtgccctgccgaccctcgtcgttttcggcttcattattacgctacttttctttctctttatgctctacttttggaacaacgacgtgttccgtaagctgcttcgctgcgcttggatccagcgctgctgcgaccgcttcgacgcgtggcaagacgaggtcatctaccgtcgtccatcacgtcgttcccagagcgacgacgagagtcgtactaacagcgtgtcatcgtacgttcttttatcacccgcgtccgatggcggttttgacaacccggcactgacagaggccgtcgacagcgtggacgactgggcaaccacctcggttttttacgccacgtccgacgaaacggcggacaccgagcgccgagactcgcagcaactgctcatcgagcttccgccggagccgctcccacccgacgtggtagcggccatgcagaaagcggtgaaacgcgccgtacagaacgcgctgcgccacagccacgactcttggcagcttcatcagaccctgtgacgcagatgaacgttccttcttaaacatccgaggtagcaatgagacaggtcgcgtaccgccggcgacgcgagagttcctgcgcggtgctggtccaccacgtcggccgcgacggcgacggcgaggaggaggcagcaaaaaagacctgcaaaaaaaccggacgctcagtcgcgggcatcccgggcgagaagctgcgtcgcacggtggtcaccaccacgccggcccgacgtttgagcggccgacacacggagcaggagcaggcgcgcagcgtctctgcgaaaaagggaaagaaagaatcatcatgtgccgccgagagtcgctccgaactctgccgtggctgttctgggtgctgttgagctgcccgcgactcctcgaatattcttcctcttcgttccccttcgccaccgctgacattgccgaaaagatgtgggccgagaattatgagaccacgtcgccggcgccggtgttggtcgccgagggagagcaagttaccatcccctgcacggtcatgacacactcctggcccatggtctccattcgcgcacgtttctgtcgttcccacgacggcagcgacgagctcatcctggacgccgtcaaaggccatcggctgatgaacggactccagtaccgcctgccgtacgccacttggaatttctcgcaattgcatctcggccaaatattctcgctgactttcaacgtatcgacggacacggccggcatgtacgaatgcgtgctgcgcaactacagccacggcctcatcatgcaacgcttcgtaattctcacgcaactggagacgctcagccggcccgacgaaccttgctgcacgccggcgttaggtcgttactcgctgggagaccagatctggtcgccgacgccctggcgtctacggaatcacgactgcgggatgtaccgcggttttcaacgcaactacttctatatcggccgcgccgacgccgaggattgctggaaacccgcatgtccggacgaggaacccgaccgctgttggacagtgatacagcgttaccggctccccggcgactgctaccgttcgcagccacacccgccgaaatttttaccggtgacgccagcaccgccggccgacatagacaccgggatgtctccctgggccactcggggaatcgcggcatttttgggattttggagtattttcaccgtatgtttcctatgctacctgtgttacctgcagtgctgtggacgctggtgccccacgccgggaaggggacgacgaggtggtgagggctatcgacgcctaccgacttacgatagttaccccggtgttaaaaagatgaagagggactacaaggatgacgacgataagtgagaacatagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggctgaagagggactacaaggatgacgacgataagtgagaacacgcataaaataaaaaaataacatgttaaaaaatgcagtgtgtgaaatgtgaatagtgtgattaaaatatgcggattgaatgggcgtgttggttattcggatactttgtgtcatccgttgggagcgaacggtcattatcctatcgttaccacctggaatctaattcatctgccaacgtggtttgtaacggaaacatttccgtgtttgtaaacggcaccctaggtgtgcgatataacattacggtaggaatcagttcgtctttattaataggacaccttactatacaaacattggagtcatggttcacatcctgggtccaaaataagagttacagcaaacaacccctaagtaccaccgaaacgctttacaatatagatagcgaaaacattcatcgcgtatctcaatattttcacacaagatggataaaatctctgcaagaaaatcacacttgcgacctcacaaacagtacacctacctatacatatcaagcaaacgtaaacaacacgaattacctaacactaacatcctcgggatggcaagaccgtctaaattataccgccataaatagtacacactttaacctcacagaatcgaacataaccagcattcacaaatatctcaacactacctgcatagaaagactccgtaactacaccttgaagtccgtatacaccacaactgtgcctcaaaacgtaacaacacctcagcacacaacaaccactctgtacacacctccaaatgcaataacaattcaagatacaactcaaagccatactgtacagacgccgtcttttaacgacacacataacgtgacggaacacacgttaaacataaactacgttttatcacaaaaaacgaataacacaacatcaccgtgggtatatgccatacctatgggcgctacagccacaataggcgccagtttatatatcggaaaacactttacgccggttaggttcgtatacgaagtatggcgcggtcagtaa

SEQ ID NO: 68 - protein, 191 aa, Artificial Sequence; identical to SEQ ID NO: 64
  Synthetic polypeptide

SEQ ID NO: 69 - protein, 70 aa, Artificial Sequence; identical to SEQ ID NO: 65
  Synthetic polypeptide

SEQ ID NO: 70 - protein, 305 aa, Artificial Sequence; identical to SEQ ID NO: 66
  Synthetic polypeptide

SEQ ID NO: 71 - DNA, 2905 bp, Artificial Sequence
  Synthetic polynucleotide
atgaccccagctcagactaacgccactaccaccgtgcacccgcacgacacaaaaaacggcagcggcggtagtgccctgccgaccctcgtcgttttcggcttcattattacgctacttttctttctctttatgctctacttttggaacaacgacgtgttccgtaagctgcttcgctgcgcttggatccagcgctgctgcgaccgcttcgacgcgtggcaagacgaggtcatctaccgtcgtccatcacgtcgttcccagagcgacgacgagagtcgtactaacagcgtgtcatcgtacgttcttttatcacccgcgtccgatggcggttttgacaacccggcactgacagaggccgtcgacagcgtggacgactgggcaaccacctcggttttttacgccacgtccgacgaaacggcggacaccgagcgccgagactcgcagcaactgctcatcgagcttccgccggagccgctcccacccgacgtggtagcggccatgcagaaagcggtgaaacgcgccgtacagaacgcgctgcgccacagccacgactcttggcagcttcatcagaccctgtgacgcagatgaacgttccttcttaaacatccgaggtagcaatgagacaggtcgcgtaccgccggcgacgcgagagttcctgcgcggtgctggtccaccacgtcggccgcgacggcgacggcgaggaggaggcagcaaaaaagacctgcaaaaaaaccggacgctcagtcgcgggcatcccgggcgagaagctgcgtcgcacggtggtcaccaccacgccggcccgacgtttgagcggccgacacacggagcaggagcaggcgcgcagcgtctctgcgaaaaagggaaagaaagaatcatcatgtgccgccgagagtcgctccgaactctgccgtggctgttctgggtgctgttgagctgcccgcgactcctcgaatattcttcctcttcgttccccttcgccaccgctgacattgccgaaaagatgtgggccgagaattatgagaccacgtcgccggcgccggtgttggtcgccgagggagagcaagttaccatcccctgcacggtcatgacacactcctggcccatggtctccattcgcgcacgtttctgtcgttcccacgacggcagcgacgagctcatcctggacgccgtcaaaggccatcggctgatgaacggactccagtaccgcctgccgtacgccacttggaatttctcgcaattgcatctcggccaaatattctcgctgactttcaacgtatcgacggacacggccggcatgtacgaatgcgtgctgcgcaactacagccacggcctcatcatgcaacgcttcgtaattctcacgcaactggagacgctcagccggcccgacgaaccttgctgcacgccggcgttaggtcgttactcgctgggagaccagatctggtcgccgacgccctggcgtctacggaatcacgactgcgggatgtaccgcggttttcaacgcaactacttctatatcggccgcgccgacgccgaggattgctggaaacccgcatgtccggacgaggaacccgaccgctgttggacagtgatacagcgttaccggctccccggcgactgctaccgttcgcagccacacccgccgaaatttttaccggtgacgccagcaccgccggccgacatagacaccgggatgtctccctgggccactcggggaatcgcggcatttttgggattttggagtattttcaccgtatgtttcctatgctacctgtgttacctgcagtgctgtggacgctggtgccccacgccgggaaggggacgacgaggtggtgagggctatcgacgcctaccgacttacgatagttaccccggtgttaaaaagatgaagagggactacaaggatgacgacgataagtgagaacacgcataaaataaaaaaataacatgttaaaaaatgcagtgtgtgaaatgtgaatagtgtgattaaaatatgcggattgaatgggcgtgttggttattcggatactttgtgtcatccgttgggagcgaacggtcattatcctatcgttaccacctggaatctaattcatctgccaacgtggtttgtaacggaaacatttccgtgtttgtaaacggcaccctaggtgtgcgatataacattacggtaggaatcagttcgtctttattaataggacaccttactatacaaacattggagtcatggttcacatcctgggtccaaaataagagttacagcaaacaacccctaagtaccaccgaaacgctttacaatatagatagcgaaaacattcatcgcgtatctcaatattttcacacaagatggataaaatctctgcaagaaaatcacacttgcgacctcacaaacagtacacctacctatacatatcaagcaaacgtaaacaacacgaattacctaacactaacatcctcgggatggcaagaccgtctaaattataccgccataaatagtacacactttaacctcacagaatcgaacataaccagcattcacaaatatctcaacactacctgcatagaaagactccgtaactacaccttgaagtccgtatacaccacaactgtgcctcaaaacgtaacaacacctcagcacacaacaaccactctgtacacacctccaaatgcaataacaattcaagatacaactcaaagccatactgtacagacgccgtcttttaacgacacacataacgtgacggaacacacgttaaacataaactacgttttatcacaaaaaacgaataacacaacatcaccgtgggtatatgccatacctatgggcgctacagccacaataggcgccagtttatatatcggaaaacactttacgccggttaggttcgtatacgaagtatggcgcggtcagtaa

SEQ ID NO: 72 - protein, 313 aa, Artificial Sequence
  Synthetic polypeptide
MKRRRRWRGWLLFLALCFCLLCEAVETNATTVTSTTAAAATTNTTVATTGTTTTSPNVTSTTSNTVITPTTVSSVSNLTSSATSIPISTSTVSGTRNTRNNNTTTIGTNVTSPSPSVSILTTVTPAATSTTSNNGDVTSDYTPTFDLENITTTRAPTRPPAQDLCSHNLSIILYEEESQSSVDIAVDEEEPELEDDDEYDELWFPLYFEAECNLNYTLQYVNHSCDYSVRQSSVSFPPWRDIDSVTFVPRNLSNCSAHGLAVIVAGNQTWYVNPFSLAHLLDAIYNVLGIEDLSANFRRQLAPYRHTLIVPQT

SEQ ID NO: 73 - protein, 278 aa, Artificial Sequence; identical to SEQ ID NO: 35
  Synthetic polypeptide

SEQ ID NO: 74 - DNA, 1138 bp, Artificial Sequence
  Synthetic polynucleotide
actggctccttaccgtcacactctcatcgtgccgcagactgagcagaagctcatctctgaagaggatctgtgatgtagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagactgagcagaagctcatctctgaagaggatctgtgatgtgccgccgcccggattgcggcttctctttctcacc

SEQ ID NO: 75 - protein, 425 aa, Artificial Sequence
  Synthetic polypeptide
MVMFSQDHVQIVYGSTRICKSLAPANKRKTHRTIVVAPRRGFLRIPPDGQDVNHVKIVPTTTSSSLAPPRDDERRPTPPLRPPLTVYPYGTSLIRRSARDAKLRSKLIVFHITRPALGQHPQNPGISGPAAMDHSEFLTSFRREVDRQTVLTAESAPATVEVCLGDALPGGVMGGGGLPAGVGSASAAVAAAAAAVAGVPVAANPVMPATATVTTPPMIDLTSHHRPLTLFTPASAAAAPAVATNGGNATYILPADCRYAPLFASKYKYVFEEVSRLMRLHDSTAVQLQISASCGNAFQALKSALLKLHNVTVLAGQQLITQTMPHTPQAVATFKFFHQDPNRVLDCIRPVVPRSTSYHETGVYQMWVSGATKKDLFDAVTLCASIVEKQPDVFNINVSLLTYPSIAAPHLPLYNEFTSFRLPTS

SEQ ID NO: 76 - protein, 278 aa, Artificial Sequence; identical to SEQ ID NO: 35
  Synthetic polypeptide

SEQ ID NO: 77 - protein, 250 aa, Artificial Sequence
  Synthetic polypeptide
MALKQWMLANIADNKGSLLTPDEQARVFCLSADWIRFLSLPDHDTVLLRDTVAAVEGARQLEMVYPAPEHVHRWSYLCPPEQVRVVIVGQDPYCDGSASGLAFGTLAGRPPPPSLNNVFRELARTVDGFQRPASGCLDAWARRGVLLLNTVFTVVHGQPGSHRHLGWQTLSNHVIRRLSERREHLVFMLWGADAHTCEYLIDRRRHLVLKSCHPSPRNTTRAFVGNDHFILANAYLDTHYRETMDWRLCG

SEQ ID NO: 78 - DNA, 3978 bp, Artificial Sequence
  Synthetic polynucleotide
atggtcatgttctcccaggaccacgtccagatcgtctacggctccacacgcatctgcaagtccctggccccggccaacaagagaaagacccatcgcaccatcgtggtagcgccgcggcggggttttttgcgtattcccccggacgggcaggacgttaaccacgtcaagatcgtacccaccaccacctcctcctccctcgcgccgccccgtgacgacgaacggcggccgaccccgccgttgcggccgccgctcacagtctatccctatggcacgtcgctgatccgcaggtcagctagggacgcaaaactccgctccaagctaatcgtcttccatataactcggcccgcactcggtcagcacccgcagaatcccgggatctcgggccctgcggccatggatcatagtgagttcctcaccagctttcgtcgcgaggtcgatcggcaaaccgtactgaccgccgagagcgcgcccgccactgtggaagtctgtctgggcgacgctctgcccggcggcgtcatgggtggcggaggactgccggcaggtgtgggctcggccagcgccgccgtggccgccgctgccgccgctgtggccggagtgcccgtcgccgcaaaccccgtcatgcccgccaccgccacggtcaccacgccgcccatgatcgaccttaccagccaccaccggccgctgacgctgtttacgccggcctccgcggccgctgcgcccgccgtggccaccaacggcggcaacgccacctacattttgccggccgactgtcggtacgcgccactctttgccagtaagtataagtacgtcttcgaagaggtgtcacgtctcatgcggctgcacgactccacggccgtgcagctgcaaatcagcgcctcgtgcggcaacgctttccaggccctcaagagtgccctgctcaagttgcacaacgtcaccgtgttagcgggccagcagctcatcacgcagacgatgccgcacacgccacaggccgtagccacgttcaaattctttcaccaggatcccaaccgcgttctggactgcatccgtccggtggtgccgcgctctacttcgtaccacgagacgggtgtataccagatgtgggtctcgggcgccaccaagaaagacctgttcgacgccgtaaccctgtgcgcttccatcgtagaaaagcagccagacgtatttaatatcaacgtcagcttgttgacgtacccttcgatcgcggcgccccacttgccgctgtacaacgaattcaccagctttcgcctgcccacctcatgaagcggcggcggcgatggcggggctggttgcttttcctggccctgtgcttttgcttactgtgtgaagcggtggaaaccaacgcgaccaccgttaccagtaccaccgctgccgccgccacgacaaacactaccgtcgccaccaccggtaccactactacctcccctaacgtcacttcaaccacgagtaacaccgtcatcactcccaccacggtttcctcggtcagcaatctgacatccagcgccacgtcgattcccatctcaacgtcaacggtttctggaacaagaaacacaaggaataataataccacaaccatcggtacgaacgttacttccccctccccttctgtatccatacttaccaccgtgacaccggccgcgacttctaccacctccaacaacggggatgtaacatccgactacactccaacttttgacctggaaaacattaccaccacccgcgctcccacgcgtcctcccgcccaggacctttgtagccataacctgtcaatcatcctgtacgaagaggaatctcagagcagcgtagacattgcggtggatgaagaagagccagaactggaggacgacgacgagtacgacgaactgtggttccccctctacttcgaggctgagtgcaacctaaattacacgctacaatacgtcaatcacagttgtgattacagcgtgcgccagtcgtctgtctcattccccccgtggcgcgacatcgactcagttaccttcgtacccaggaacctctccaactgtagcgcccacggtctggccgtcatcgtcgcgggtaaccaaacctggtacgtgaatccgtttagcctggctcacctgctggatgcaatatataacgttttagggatcgaagacctgagcgccaactttcggcgccaactggctccttaccgtcacactctcatcgtgccgcagactgagcagaagctcatctctgaagaggatctgtgatgtgccgccgcccggattgcggcttctctttctcacctggaccggtggtactgctgtggtgttgccttctgctgcccattgtttcctcagtcgccgtcagcgtcgctcctaccgccgccgagaaagtccccgcggagtgtcccgaactaacgcgccgatgcctgttgggtgaggtgtttcagggtgacaagtatgaaagttggctgcgtccgttggtgaatgttaccgggcgcaatggcccgctatcgcaacttatccgttaccgtcccgttacgccggaggccgccaactccgtgctgttggacgatgctttcctggacactctggccctgctgtacaacaatccggatcaattgcgggccctgctgacactgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattttgcctgcgtcaccagctggacccgccgctgctacgccacctagataaatactacgccggactgccgcccgagctgaagcagacgcgcgtcaacctgccggctcactcgcgctatggccctcaagcagtggatgctcgctaacattgctgataataaaggctctctgttaacccccgacgagcaggctcgcgtgttttgtctgagcgccgactggatccgcttcctcagcttgcccgaccacgacacggtgctgctgcgcgacacggtggcggcggtggagggcgcgcggcaactcgagatggtgtacccggcacccgagcacgtacaccgctggagctatctgtgtccgcccgagcaggtgcgcgtggtgatcgtgggccaggacccgtactgcgacggcagcgccagcggcctggccttcggcacgctggcaggccgaccaccgccgccctccctcaacaacgtgtttcgcgaactggcgcgtacggtggacgggtttcagcgtccggcctcgggttgcctggacgcctgggcgcgacgcggcgtgctgctgctcaacaccgtgttcaccgtggtgcacggacagcctggatctcaccgtcacttaggctggcagacgctgagtaaccatgtgatccggaggctgtcagagcgccgggagcatctggtgttcatgctgtggggcgccgacgcgcacacctgcgagtacctgatcgaccgacggcggcacttggtactcaagtcctgccacccgtcgccgcgcaacaccacccgcgccttcgtgggcaacgaccactttatactggccaacgcgtatctggacacgcattaccgagagaccatggactggcgactctgtgggtgattgatgataatagacacaccggggagacaaggtggtgaactgtgatgtctatctctttattgtaaaaaaaaatcagtgaccacaatatatatagacacacaccgaaaggggtggaaacccaaatgtccaagcgcgtcctgtttcataatttttccggtctcggctctgtttt

SEQ ID NO: 79 - protein, 191 aa, Artificial Sequence; identical to SEQ ID NO: 64
  Synthetic polypeptide

SEQ ID NO: 80 - protein, 70 aa, Artificial Sequence; identical to SEQ ID NO: 65
  Synthetic polypeptide

SEQ ID NO: 81 - protein, 305 aa, Artificial Sequence; identical to SEQ ID NO: 66
  Synthetic polypeptide

SEQ ID NO: 82 - DNA, 3933 bp, Artificial Sequence; identical to SEQ ID NO: 67
  Synthetic polynucleotide

SEQ ID NO: 83 - DNA, 1132 bp, Artificial Sequence; identical to SEQ ID NO: 63
  Synthetic polynucleotide

SEQ ID NO: 84 - protein, 70 aa, Artificial Sequence; identical to SEQ ID NO: 65
  Synthetic polypeptide

SEQ ID NO: 85 - protein, 70 aa, Artificial Sequence; identical to SEQ ID NO: 65
  Synthetic polypeptide

SEQ ID NO: 86 - DNA, 1107 bp, Artificial Sequence
  Synthetic polynucleotide
gtcgccggcgccggtgttggtcgccgagggagagcaagttaccatcccctgcacggtcatagggataacagggtaatcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggctcgccgagggagagcaagttaccatcccctgcacggtcatgacacactcctggcccatgg

SEQ ID NO: 87 - protein, 425 aa, Artificial Sequence; identical to SEQ ID NO: 75
  Synthetic polypeptide

SEQ ID NO: 88 - protein, 313 aa, Artificial Sequence; identical to SEQ ID NO: 72
  Synthetic polypeptide

SEQ ID NO: 89 - protein, 278 aa, Artificial Sequence; identical to SEQ ID NO: 35
  Synthetic polypeptide

SEQ ID NO: 90 - protein, 249 aa, Artificial Sequence
  Synthetic polypeptide
MALKQWMLANIADNKGSLLTPDEQARVFCLSADWIRFLSLPDHDTVLLRDTVAAVEGARQLEMVYPAPEHVHRWSYLCPPEQVRVVIVGQDPYCDGSASGLAFGTLAGRPPPPSLNNVFRELARTVDGFQRPASGCLDAWARRGVLLLNTVFTVVHGQPGSHRHLGWQTLSNHVIRRLSERREHLVFMLWGADAHTCEYLIDRRRHLVLKSCHPSPRNTTRAFVGNDHFILANAYLDTHYRETMDWRLC

SEQ ID NO: 91 - DNA, 3978 bp, Artificial Sequence; identical to SEQ ID NO: 78
  Synthetic polynucleotide

SEQ ID NO: 92 - protein, 70 aa, Artificial Sequence; identical to SEQ ID NO: 65
  Synthetic polypeptide

SEQ ID NO: 93 - DNA, 83 bp, Artificial Sequence
  Synthetic polynucleotide
ccatgggccaggagtgtgtcatgaccgtgcaggggatggtaacttgctctccctcggcgagccagtgttacaaccaattaacc

SEQ ID NO: 94 - DNA, 83 bp, Artificial Sequence
  Synthetic polynucleotide
gtcgccggcgccggtgttggtcgccgagggagagcaagttaccatcccctgcacggtcatagggataacagggtaatcgattt

SEQ ID NO: 95 - DNA, 95 bp, Artificial Sequence
  Synthetic polynucleotide
ttttttaacatgttatttttttattttatgcgtgttctcacttatcgtcgtcatccttgtagtccctcttcagccagtgttacaaccaattaacc

SEQ ID NO: 96 - DNA, 96 bp, Artificial Sequence
  Synthetic polynucleotide
gacttacgatagttaccccggtgttaaaaagatgaagagggactacaaggatgacgacgataagtgagaacatagggataacagggtaatcgattt

SEQ ID NO: 97 - protein, 278 aa, Artificial Sequence; identical to SEQ ID NO: 35
  Synthetic polypeptide

SEQ ID NO: 98 - protein, 313 aa, Artificial Sequence; identical to SEQ ID NO: 72
  Synthetic polypeptide

SEQ ID NO: 99 - DNA, 98 bp, Artificial Sequence
  Synthetic polynucleotide
ggtgagaaagagaagccgcaatccgggcggcggcacatcacagatcctcttcagagatgagcttctgctcagtctgccagtgttacaaccaattaacc

SEQ ID NO: 100 - DNA, 99 bp, Artificial Sequence
  Synthetic polynucleotide
actggctccttaccgtcacactctcatcgtgccgcagactgagcagaagctcatctctgaagaggatctgtgatgtagggataacagggtaatcgattt

SEQ ID NO: 101 - protein, 18 aa, Artificial Sequence
  Synthetic polypeptide
MKWVTFISLLFLFSSAYS

SEQ ID NO: 102 - protein, 6 aa, Artificial Sequence
  Synthetic polypeptide
GSGGGS

SEQ ID NO: 103 - protein, 15 aa, Artificial Sequence
  Synthetic polypeptide
KETAAAKFERQHMDS

SEQ ID NO: 104 - protein, 14 aa, Artificial Sequence
  Synthetic polypeptide
GGSGGSDYKDDDDK

SEQ ID NO: 105 - protein, 8 aa, Artificial Sequence
  Synthetic polypeptide
DYKDDDDK

SEQ ID NO: 106 - protein, 16 aa, Artificial Sequence
  Synthetic polypeptide
GGSGGSEQKLISEEDL

SEQ ID NO: 107 - protein, 10 aa, Artificial Sequence
  Synthetic polypeptide
EQKLISEEDL